(19) United States
(12) Patent Application Publication
(10) Pub. No.: US 2025/0032484 A1
(43) Pub. Date: Jan. 30, 2025

(54) THERAPEUTIC TARGETS AND AGENTS FOR THE TREATMENT OF POSTTRAUMATIC STRESS DISORDER

(71) Applicants: The United States Government as represented by the Department of Veterans Affairs, Washington, DC (US); MARY HITHCOCK MEMORIAL HOSPITAL, Hanover, NH (US); THE TRUSTEES OF BOSTON UNIVERSITY, Boston, MA (US)

(72) Inventors: Brian Shiner, Norwich, VT (US); Jaimie L. Gradus, Braintree, MA (US)

(21) Appl. No.: 18/715,993

(22) PCT Filed: Dec. 2, 2022
(86) PCT No.: PCT/US2022/080832

§ 371 Date: Jun. 3, 2024

Related U.S. Application Data
(60) Provisional application No. 63/285,841, filed on Dec. 3, 2021.

(51) Int. Cl. A61K 31/4985; A61K 31/4188; A61K 31/439; A61K 31/454; A61K 31/7072; A61K 45/06; A61P 25/00; A61P 25/32

(52) U.S. Cl. CPC A61K 31/4985; A61K 31/4188; A61K 31/439; A61K 31/454; A61K 31/7072; A61K 45/06; A61P 25/00; A61P 25/32

(57) ABSTRACT

The invention generally relates to methods of treating psychiatric disorders using one or more compounds selected from pibrentasvir, glecaprevir, velpatasvir, ledipasvir, and sofosbuvir, or pharmaceutically acceptable salts thereof, and pharmaceutical compositions comprising same. This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/285,841, filed on Dec. 3, 2021, the contents of which are incorporated herein by reference in their entirety.

### STATEMENT REGARDING FEDERALLY SPONSORED RESEARCH

This invention was made with government support under grant number R01MH121397 awarded by the National Institute of Mental Health, and grant number PR160206 awarded by the Department of Defense. The government has certain rights in the invention.

### BACKGROUND

Posttraumatic stress disorder (PTSD) is the fifth most prevalent mental disorder in the United States (Kessler et al. (2005) Arch Gen Psychiatry 62 (6): 617-27), yet there are only two US Food and Drug Administration (FDA) medications approved for PTSD, and they have limited effectiveness in reducing symptoms and improving functioning (Krystal et al. (2017) Biol Psychiatry 82 (7): e51-e59). Thus, there is interest in developing novel agents that target mechanisms involved in PTSD pathophysiology (DePierro et al. (2019) Biol Psychiatry 86 (6): 454-63: Sartori and Singewald (2019) Pharmacol Ther. 204:107402). At the same time, some existing medications may have the potential to ameliorate PTSD symptoms (Schoenfeld et al. (2004) Psychiatr Serv. 55 (5): 519-31: Yehuda et al. (2015) Nat Rev Dis Primers 1:15057). Several of these medications have been recently tested in randomized clinical trials (RCTs) as their mechanism of action aligns with known PTSD-related pathophysiologic deficits (Raskind et al. (2018) N Engl J Med. 378 (6): 507-17: Feder et al. (2021) Am J Psychiatry 178 (2): 193-202: Spangler et al. (2020) J Clin Psychiatry 81 (6)). However, the pathophysiology of PTSD is incompletely understood (Yehuda et al. (2015) Nat Rev Dis Primers 1:15057), and there are over a thousand FDA-approved medications affecting an expansive range of known and unknown biological targets (Ma'ayan et al. (2007) Mt Sinai J Med. 74 (1): 27-32: Barneh et al. (2016) Brief Bioinform. 17 (6): 1070-80). Thus, a therapeutic discovery approach that matches known PTSD pathophysiology to medications' known mechanisms of action may overlook potentially effective treatments. Real-world studies of the association between medication receipt and PTSD symptom change may suggest additional treatments (Gordon et al. (2017) Biol Psychiatry 82 (7): 460-61), and these intersections of disease and treatment may suggest unrecognized pathophysiology and therapeutic targets. For at least the foregoing reasons, there remains a need for compounds and compositions for treating psychiatric disorders such as PTSD, and methods of making and using same.

### SUMMARY

In accordance with the purpose(s) of the invention, as embodied and broadly described herein, the invention, in one aspect, relates to methods of treating psychiatric disorders using one or more compounds selected from pibrentasvir, glecaprevir, velpatasvir, ledipasvir, and sofosbuvir, or pharmaceutically acceptable salts thereof.

Disclosed are methods of treating a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the subject has not been diagnosed as having hepatitis C virus.

Also disclosed are methods of reducing the severity of one or more symptoms of a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the subject has not been diagnosed as having hepatitis C virus.

Also disclosed are methods of treating a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the psychiatric disorder is selected from attention-deficit/hyperactivity disorder, autism spectrum disorder, conduct disorder, disruptive mood dysregulation disorder, an eating disorder, gender dysphoria, internet gaming disorder, obsessive-compulsive disorder, a paraphilic disorder, a personality disorder, posttraumatic stress disorder, a sleep-wake disorder, a specific learning disorder, a social communication disorder, and a somatic symptom disorder.

Also disclosed are methods of treating a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the psychiatric disorder is one or more selected from attention-deficit/hyperactivity disorder, autism spectrum disorder, conduct disorder, disruptive mood dysregulation disorder, an eating disorder, gender dysphoria, internet gaming disorder, obsessive-compulsive disorder, a paraphilic disorder, a personality disorder, posttraumatic stress disorder, a sleep-wake disorder, a specific learning disorder, a social communication disorder, a substance-use disorder, and a somatic symptom disorder.

Also disclosed are methods of treating a trauma- or stressor-related disorder in a subject in need thereof, the method comprising administering to the subject an effective amount of pibrentasvir and glecaprevir, or pharmaceutically acceptable salts thereof.

Also disclosed are methods of treating an anxiety disorder in a subject in need thereof, the method comprising administering to the subject an effective amount of one or more compounds selected from pibrentasvir, glecaprevir, and velpatasvir, or pharmaceutically acceptable salts thereof.

Also disclosed are methods of treating a substance-use disorder in a subject in need thereof, the method comprising administering to the subject an effective amount of one or more compounds selected from pibrentasvir, glecaprevir, velpatasvir, ledipasvir, and sofosbuvir, or pharmaceutically acceptable salts thereof, wherein the subject has not been diagnosed as having hepatitis C virus.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows a representative schematic illustrating the mechanistic TreeScan results for antiviral medications: associations with improvement in PTSD symptoms. O:E=Observed Cases over Expected Case. Statistical alerts (P<0.1) are highlighted in blue.

FIG. 2 shows a representative schematic illustrating the identification of the analytic cohort (DAA=Direct-Acting Antiviral: HCV=Hepatitis C Virus: PCL=PTSD Checklist).

FIG. 3 shows a representative diagram illustrating the timing of alcohol use disorders identification test measurements in relation to exposure to direct acting antiviral medications and HCV cure.

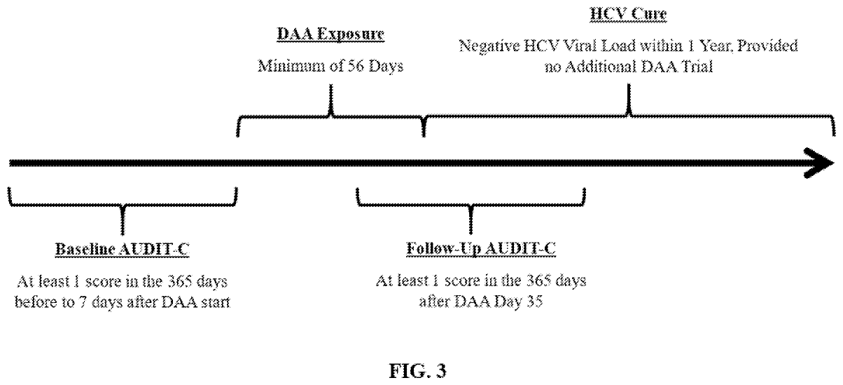

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group.” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and new born subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step. In various aspects, the one or more disorders are a psychiatric disorder (e.g., posttraumatic stress disorder).

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder: preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it: (ii) inhibiting the disease, i.e., arresting its development: or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of a viral infection prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically: that is, administered to treat an existing disease or condition. In further various aspects, a preparation can be administered prophylactically: that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder: the specific composition employed: the age, body weight, general health, sex and diet of the patient: the time of administration; the route of administration: the rate of excretion of the specific compound employed: the duration of the treatment: drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”: that is, an amount effective for prevention of a disease or condition.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance.

As used herein, “EC₅₀” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein may comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below; by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules that owe their stability through non-covalent interaction. One or more components of this molecular complex provide a stable framework in the crystalline lattice. In certain instances, the guest molecules are incorporated in the crystalline lattice as anhydrates or solvates, see e.g., Almarasson, O., et al. (2004) The Royal Society of Chemistry, 1889-1896. Examples of co-crystals include p-toluenesulfonic acid and benzenesulfonic acid.

It is known that chemical substances form solids that are present in different states of order that are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991): Rodd's Chemistry of Carbon Compounds, Volumes 1-5 and Supplementals (Elsevier Science Publishers, 1989): Organic Reactions, Volumes 1-40 (John Wiley and Sons, 1991): March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow: plain meaning derived from grammatical organization or punctuation; and the number or type of embodiments described in the specification.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. METHODS FOR TREATING OR REDUCING THE SEVERITY OF ONE OR MORE SYMPTOMS OF PSYCHIATRIC DISORDERS

In one aspect, disclosed are methods of treating a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the subject has not been diagnosed as having hepatitis C virus.

In one aspect, disclosed are methods of treating a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the psychiatric disorder is selected from attention-deficit/hyperactivity disorder, autism spectrum disorder, conduct disorder, disruptive mood dysregulation disorder, an eating disorder, gender dysphoria, internet gaming disorder, obsessive-compulsive disorder, a paraphilic disorder, a personality disorder, posttraumatic stress disorder, a sleep-wake disorder, a specific learning disorder, a social communication disorder, and a somatic symptom disorder.

In one aspect, disclosed are methods of treating a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the psychiatric disorder is selected from attention-deficit/hyperactivity disorder, autism spectrum disorder, conduct disorder, disruptive mood dysregulation disorder, an eating disorder, gender dysphoria, internet gaming disorder, obsessive-compulsive disorder, a paraphilic disorder, a personality disorder, posttraumatic stress disorder, a sleep-wake disorder, a specific learning disorder, a social communication disorder, a substance-use disorder, and a somatic symptom disorder.

In one aspect, disclosed are methods of treating a trauma- or stressor-related disorder in a subject in need thereof, the method comprising administering to the subject an effective amount of pibrentasvir and glecaprevir, or pharmaceutically acceptable salts thereof.

In one aspect, disclosed are methods of reducing the severity of one or more symptoms of a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the subject has not been diagnosed as having hepatitis C virus. Examples of symptoms of a psychiatric disorder include, but are not limited to, irritability, anger, sadness, depression, and dysphoria.

Pibrentasvir is a direct acting antiviral agent and Hepatitis C virus NS5A inhibitor with a molecular formula C₅₇H₆₅F₅N₁₀O₈ and a molecular weight of 1113.2. It is approved by the U.S. Food and Drug Administration (FDA) for use with glecaprevir as a combination drug under the tradename Mavyret® for treatment of hepatitis C. Pibrentasvir has a CAS No. 1353900-92-1, and is also referred to as ABT-530 and ABT 530, among others. The structure of pibrentasvir is shown below.

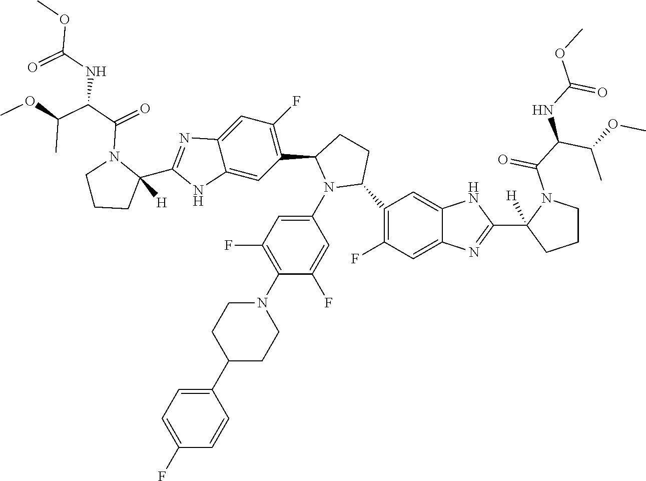

Glecaprevir is a direct acting antiviral agent and Hepatitis C virus NS3/4A protease inhibitor with a molecular formula C₃₈H₄₆F₄N₆O₉S and a molecular weight of 838.9. Glevaprevir has a CAS No. 1365970-03-1, and is also referred to as ABT-493 and UNII-K6BUU8J72P among others. The structure of glecaprevir is shown below.

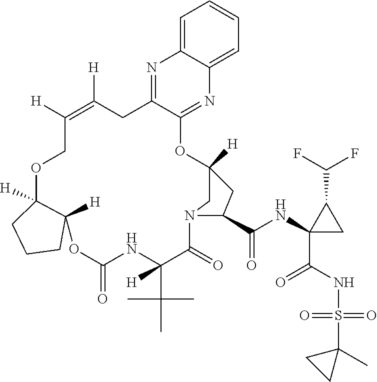 [SMILES: CC(C)(C)[C@@H]1NC(=O)O[C@@H]2CCC[C@H]2OC/C=C\Cc2nc3ccccc3nc2O[C@@H]2C[C@@H](C(=O)N[C@]3(C(=O)NS(=O)(=O)C4(C)CC4)C[C@H]3C(F)F)N(C2)C1=O]

Similar to pibrentasvir, velpatasvir is also a Hepatitis C virus NS5A inhibitor. It is approved by the FDA as a fixed-dose combination tablet of velpatasvir and sofosbuvir under the tradename Epclusa® for treatment of hepatitis C in adults. Velpatasvir has a molecular formula C₄₉H₅₄N₈O₈ and a molecular weight of 883.0. Velpatasvir has a CAS No. 1377049-84-7, and is also referred to as GS-5816, GS5816, and UNII-KCU0C7RS7Z, among others. The structure of velpatasvir is shown below.

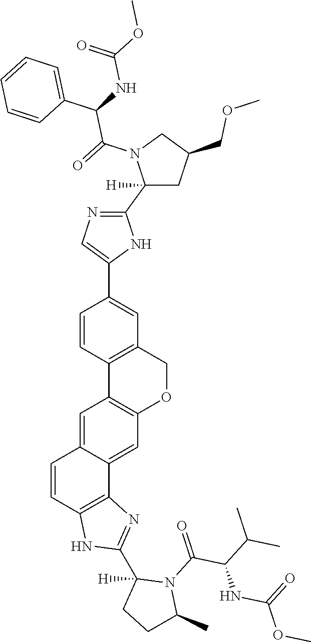 [SMILES: COC[C@H]1C[C@@H](c2ncc(-c3ccc4c(c3)COc3cc5c(ccc6[nH]c([C@@H]7CC[C@H](C)N7C(=O)[C@@H](NC(=O)OC)C(C)C)nc65)cc3-4)[nH]2)N(C(=O)[C@H](NC(=O)OC)c2ccccc2)C1]

Sofosbuvir is a Hepatitis C virus NS5B inhibitor. In addition to being FDA approved in combination with velpatasvir, sofosbuvir is also approved in combination with ledipasvir as fixed-dose combination tablet under the tradename Harvoni® for treatment of genotype 1 hepatitis C. Sofosbuvir has a molecular formula C₂₂H₂₉FN₃O₉P, a molecular weight of 529.458 g/mol, and a CAS No. 1190307-88-0. The structure of sofosbuvir is shown below.

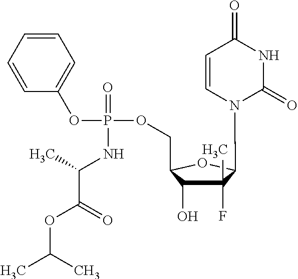

Another exemplary Hepatitis C virus NS5A inhibitor is ledipasvir. Ledipasvir has a molecular formula C₄₉H₅₄F₂N₈O₆, a molecular weight of 889.018 g/mol, and a CAS No. 1256388-51-8. The structure of ledipasvir is shown below.

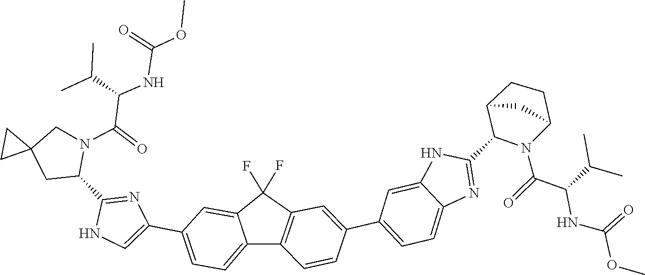 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a psychiatric disorder, such as, for example, a trauma- or stressor-related disorder (e.g., posttraumatic stress disorder) or a substance-use disorder (e.g., alcohol use disorder).

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, intrathecal administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically: that is, administered to treat an existing disease or condition. A preparation can also be administered prophylactically: that is, administered for prevention of a psychiatric disorder, such as, for example, a trauma- or stressor-related disorder (e.g., posttraumatic stress disorder) or a substance-use disorder (e.g., alcohol use disorder).

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

In a further aspect, the method comprises administering an individually effective amount of one or more compounds selected from pibrentasvir, glecaprevir, and velpatasvir, or pharmaceutically acceptable salts thereof. For example, in various aspects, the method comprises administering an individually effective amount of pibrentasvir or a pharmaceutically acceptable salt thereof. In a further aspect, the method comprises administering an individually effective amount of glecaprevir or a pharmaceutically acceptable salt thereof. In a still further aspect, the method comprises administering an individually effective amount of velpatasvir or a pharmaceutically acceptable salt thereof.

In a further aspect, the method comprises administering a synergistically effective amount of two or more compounds selected from pibrentasvir, glecaprevir, and velpatasvir, or pharmaceutically acceptable salts thereof. For example, in various aspects, the method comprises administering a synergistically effective amount of pibrentasvir and glecaprevir, or pharmaceutically acceptable salts thereof. In a further aspect, the method comprises administering a synergistically effective amount of pibrentasvir and velpatasivir, or pharmaceutically acceptable salts thereof. In a still further aspect, the method comprises administering a synergistically effective amount of glecaprevir and velpatasvir, or pharmaceutically acceptable salts thereof. In yet a further aspect, the method comprises administering a synergistically effective amount of pibrentasvir, glecaprevir, and velpatasvir, or pharmaceutically acceptable salts thereof.

In a further aspect, the method comprises administering exactly one of pibrentasvir, glecaprevir, and velpatasvir, or pharmaceutically acceptable salts thereof. For example, in various aspects, the method comprises administering pibrentasvir or a pharmaceutically acceptable salt thereof. In a further aspect, the method comprises administering glecaprevir or a pharmaceutically acceptable salt thereof. In a still further aspect, the method comprises administering velpatasvir or a pharmaceutically acceptable salt thereof.

In a further aspect, the method comprises administering two of pibrentasvir, glecaprevir, and velpatasvir, or pharmaceutically acceptable salts thereof. For example, in various aspects, the method comprises administering pibrentasvir and glecaprevir, or pharmaceutically acceptable salts thereof. In a further aspect, the method comprises administering pibrentasvir and velpatasvir, or pharmaceutically acceptable salts thereof. In a still further aspect, the method comprises administering glecaprevir and velpatasvir, or pharmaceutically acceptable salts thereof.

In a further aspect, the compounds are administered in a ratio of from about 1:10 to about 4:7, from about 1:8 to about 3:6, from about 1:7 to about 2:6, or from about 1:6 to about 1:3, or from about 1:6 to about 1:2. In a still further aspect, the compounds are administered in a ratio of about 1:10, about 1:5, about 3:10, about 2:5, about 1:2, about 3:5, about 7:10, about 4:5, or about 9:10. In yet a further aspect, the compounds are administered in a ratio of from about 4:7, about 3:7, about 2:7, or about 1:7. In an even further aspect, the compounds are administered in a ratio of about 2:5.

In a further aspect, the ratio of pibrentasvir to glecaprevir is of from about 1:10 to about 4:7, from about 1:8 to about 3:6, from about 1:7 to about 2:6, or from about 1:6 to about 1:3, or from about 1:6 to about 1:2. In a still further aspect, the ratio of pibrentasvir to glecaprevir is of about 1:10, about 1:5, about 3:10, about 2:5, about 1:2, about 3:5, about 7:10, about 4:5, or about 9:10. In yet a further aspect, the ratio of pibrentasvir to glecaprevir is of from about 4:7, about 3:7, about 2:7, or about 1:7. In an even further aspect, the ratio of pibrentasvir to glecaprevir is about 2:5.

In a further aspect, the ratio of glecaprevir to pibrentasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of glecaprevir to pibrentasvir is of about 1:10, about 1:5, about 3:10, about 2:5, about 1:2, about 3:5, about 7:10, about 4:5, or about 9:10. In yet a further aspect, the ratio of glecaprevir to pibrentasvir is of from about 4:7, about 3:7, about 2:7, or about 1:7. In an even further aspect, the ratio of glecaprevir to pibrentasvir is about 2:5.

In a further aspect, the ratio of pibrentasvir to velpatasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of pibrentasvir to velpatasvir is of about 1:10, about 1:5, about 3:10, about 2:5, about 1:2, about 3:5, about 7:10, about 4:5, or about 9:10. In yet a further aspect, the ratio of pibrentasvir to velpatasvir is of from about 4:7, about 3:7, about 2:7, or about 1:7. In an even further aspect, the ratio of pibrentasvir to velpatasvir is about 2:5.

In a further aspect, the ratio of velpatasvir to pibrentasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of velpatasvir to pibrentasvir is of about 1:10, about 1:5, about 3:10, about 2:5, about 1:2, about 3:5, about 7:10, about 4:5, or about 9:10. In yet a further aspect, the ratio of velpatasvir to pibrentasvir is of from about 4:7, about 3:7, about 2:7, or about 1:7. In an even further aspect, the ratio of velpatasvir to pibrentasvir is about 2:5.

In a further aspect, the ratio of velpatasvir to glecaprevir is of from about 1:10 to about 4:7, from about 1:8 to about 3:6, from about 1:7 to about 2:6, or from about 1:6 to about 1:3, or from about 1:6 to about 1:2. In a still further aspect, the ratio of velpatasvir to glecaprevir is of about 1:10, about 1:5, about 3:10, about 2:5, about 1:2, about 3:5, about 7:10, about 4:5, or about 9:10. In yet a further aspect, the ratio of velpatasvir to glecaprevir is of from about 4:7, about 3:7, about 2:7, or about 1:7. In an even further aspect, the ratio of velpatasvir to glecaprevir is about 2:5.

In a further aspect, the ratio of glecaprevir to velpatasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of glecaprevir to velpatasvir is of about 1:10, about 1:5, about 3:10, about 2:5, about 1:2, about 3:5, about 7:10, about 4:5, or about 9:10. In yet a further aspect, the ratio of glecaprevir to velpatasvir is of from about 4:7, about 3:7, about 2:7, or about 1:7. In an even further aspect, the ratio of glecaprevir to velpatasvir is about 2:5.

In a further aspect, the method comprises administering pibrentasvir, glecaprevir, and velpatasvir, or pharmaceutically acceptable salts thereof.

In a further aspect, when more than one compound is administered, the compounds can be administered sequentially. For example, in various aspects, pibrentasvir and glecaprevir are administered sequentially. In a further aspect, pibrentasvir and velpatasvir are administered sequentially. In a still further aspect, glecaprevir and velpatasvir are administered sequentially.

In a further aspect, when more than one compound is administered, the compounds can be administered simultaneously. For example, in various aspects, pibrentasvir and glecaprevir are administered simultaneously. In a further aspect, pibrentasvir and velpatasvir are administered simultaneously. In a still further aspect, glecaprevir and velpatasvir are administered simultaneously.

In a further aspect, when more than one compound is administered, the compounds can be co-formulated. For example, in various aspects, pibrentasvir and glecaprevir are co-formulated. In a further aspect, pibrentasvir and velpatasvir are co-formulated. In a still further aspect, glecaprevir and velpatasvir are co-formulated.

In various further aspects, the compounds can be co-formulated as an oral dosage form such as, for example, a tablet or oral pellets. For example, in various aspects, pibrentasvir and glecaprevir are co-formulated as an oral dosage form. In a further aspect, pibrentasvir and velpatasvir are co-formulated as an oral dosage form. In a still further aspect, glecaprevir and velpatasvir are co-formulated as an oral dosage form.

In various further aspects, the compounds can be co-formulated as a single oral dosage form. For example, in various aspects, pibrentasvir and glecaprevir are co-formulated as a single oral dosage form. In a further aspect, pibrentasvir and velpatasvir are co-formulated as a single oral dosage form. In a still further aspect, glecaprevir and velpatasvir are co-formulated as a single oral dosage form.

In a further aspect, the one or more compounds are administered via oral, sublingual, topical, rectal, transdermal, injection (e.g., intravenous, intramuscular), or nasal administration. In a still further aspect, the one or more compounds are administered via oral administration.

In a further aspect, the method is for treating a psychiatric disorder. Examples of psychiatric disorders include, but are not limited to, neurodevelopmental disorders (e.g., attention deficit/hyperactivity disorder, autism spectrum disorder, cerebral palsy), schizophrenia spectrum disorders (e.g., schizophrenia, schizoaffective disorder, delusional disorder, schizoptypal personality disorder, schizophreniform disorder), depressive disorders (e.g., bipolar disorder, major depression, persistent depressive disorder, seasonal affective disorder, psychotic depression, peripartum depression, treatment resistant depression, atypical depression, premenstrual dysphoric disorder), anxiety disorders (e.g., generalized anxiety disorder, panic disorder, social anxiety disorder, separation anxiety disorder, agoraphobia), obsessive-compulsive disorder, trauma- or stressor-related disorders (posttraumatic stress disorder, acute stress disorder, an adjustment disorder, reactive attachment disorder, disinhibited social engagement disorder), dissociative disorders (e.g., dissociative amnesia, dissociative fugue, depersonalization disorder, dissociative identity disorder), somatic symptom disorder, feeding or eating disorders (e.g., bulimia nervosa, anorexia nervosa, avoidant/restrictive food intake disorder), elimination disorders (e.g., encopresis, enuresisis), sleep-wake disorders (e.g., insomnia, obstructive sleep apnea, parasomnia, narcolepsy, restless leg syndrome), sexual dysfunctions or disorders (e.g., a desire disorder, an arousal disorder, an orgasm disorder, a pain disorder), gender dysphoria, disruptive, impulse-control, and conduct disorders (e.g., oppositional defiant disorder, intermittent explosive disorder, conduct disorder, pyromania, kleptomania), substance-related and addictive disorders (e.g., a substance-induced disorder, a substance-use disorder (e.g., alcohol use disorder), substance-induced psychosis, withdrawal, substance-induced delirium), neurocognitive disorders (e.g., Alzheimer's disease, vascular dementia, dementia with Lewy body, frontotemporal lobar degeneration), personality disorders (e.g., borderline personality disorder, antisocial personality disorder, histrionic personality disorder, narcissistic personality disorder, obsessive-compulsive personality disorder, schizoid personality disorder, schizotypal personality disorder), and paraphilic disorders (e.g., exhibitionistic disorder, fetishistic disorder, frotteuristic disorder, pedophilic disorder, sexual masochism disorder, sexual sadism disorder, transvestic disorder, voyeuristic disorder).

In a further aspect, the psychiatric disorder is a trauma- or stressor-related disorder. In a still further aspect, the trauma- or stressor-related disorder is selected form posttraumatic stress disorder, acute stress disorder, an adjustment disorder, reactive attachment disorder, and disinhibited social engagement disorder. In yet a further aspect, the trauma- or stressor-related disorder is posttraumatic stress disorder.

In a further aspect, the psychiatric disorder is a substance-use disorder. In a still further aspect, the substance-use disorder is alcohol use disorder.

In a further aspect, the psychiatric disorder is a co-morbid disorder. In a still further aspect, the co-morbid disorder is posttraumatic stress disorder and alcohol use disorder.

In a further aspect, the subject has not been diagnosed as having hepatitis C virus. In a still further aspect, the subject has not been diagnosed as having hepatitis C virus within the last 7 days. In yet a further aspect, the subject has not been diagnosed as having hepatitis C virus within the last 14 days. In an even further aspect, the subject has not been diagnosed as having hepatitis C virus within the last month. In a still further aspect, the subject has not been diagnosed as having hepatitis C virus within the last six months.

In a further aspect, the subject has not been diagnosed as having an infection due to a flavivirus. In a still further aspect, the subject has not been diagnosed as having an infection due to a flavivirus within the last 7 days. In yet a further aspect, the subject has not been diagnosed as having an infection due to a flavivirus within the last 14 days. In an even further aspect, the subject has not been diagnosed as having an infection due to a flavivirus within the last month. In a still further aspect, the subject has not been diagnosed as having an infection due to a flavivirus within the last six months.

In a further aspect, the subject is not currently undergoing treatment for hepatitis C virus. In a still further aspect, the subject has not previously undergone treatment for hepatitis C virus within the last 7 days. In yet a further aspect, the subject has not previously undergone treatment for hepatitis C virus within the last 14 days. In an even further aspect, the subject has not previously undergone treatment for hepatitis C virus within the last month. In a still further aspect, the subject has not previously undergone treatment for hepatitis C virus within the last six months.

In a further aspect, the subject is not currently undergoing treatment for an infection due to a flavivirus. In a still further aspect, the subject has not previously undergone treatment for an infection due to a flavivirus within the last 7 days. In yet a further aspect, the subject has not previously undergone treatment for an infection due to a flavivirus within the last 14 days. In an even further aspect, the subject has not previously undergone treatment for an infection due to a flavivirus within the last month. In a still further aspect, the subject has not previously undergone treatment for an infection due to a flavivirus within the last six months.

In a further aspect, the subject has been diagnosed with a need for treatment of the psychiatric disorder under Diagnostic and Statistical Manual of Mental Disorders (DSM-V) guidelines.

In a further aspect, the subject is a mammal. In a still further aspect, the mammal is a human. In yet a further aspect, the human is an adult (e.g., 18 years of age or older). In a still further aspect, the human is a child (e.g., 3-17 years of age).

In a further aspect, the subject has been diagnosed with a need for treatment of the psychiatric disorder prior to the administering step. In a still further aspect, the method further comprises the step of identifying a subject in need of treatment of a psychiatric disorder.

In a further aspect, the subject has been diagnosed with a need for treatment of posttraumatic stress disorder prior to the administering step. In a still further aspect, the subject has been diagnosed with a need for treatment of alcohol use disorder prior to the administering step. In yet a further aspect, the subject has been diagnosed with a need for treatment of both posttraumatic stress disorder and alcohol use disorder prior to the administering step.

In a further aspect, the effective amount is a therapeutically effective amount. In a still further aspect, the effective amount is a prophylactically effective amount.

In a further aspect, the effective amount is an amount of from about 0.1 mg/kg to about 0.4 mg/kg, about 0.1 mg/kg to about 0.3 mg/kg, about 0.1 mg/kg to about 0.2 mg/kg, about 0.2 mg/kg to about 0.4 mg/kg, about 0.3 mg/kg to about 0.4 mg/kg, or about 0.2 mg/kg to about 0.3 mg/kg. In a still further aspect, the effective amount is an amount of about 0.1 mg/kg, about 0.15 mg/kg, about 0.2 mg/kg, about 0.25 mg/kg, about 0.3 mg/kg, about 0.35 mg/kg, or about 0.4 mg/kg. In yet a further aspect, the effective amount is an amount of about 0.25 mg/kg.

In a further aspect, the effective amount is administered once per day. In a still further aspect, the effective amount is administered once per day for a period of at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days. In yet a further aspect, the effective amount is administered once per day for a period of at least 7 days. In an even further aspect, the effective amount is administered once per day for a period of at least 14 days.

In a further aspect, administering is via systemic administration.

In a further aspect, the method further comprises administering to the subject an effective amount of an agent known to treat a psychiatric disorder. Examples of agents known to treat spasticity include, but are not limited to, methylphenidate, amphetamine, atomoxetine, guanfacine, clonidine, fluoxetine, sertraline, fluvoxamine, paroxetine, citalopram, escitalopram, imipramine, desipramine, amitryptyline, nortriptyline, doxepin, clomipramine, bupropion, venlafaxine, mirtazapine, duloxetine, phenelzine, tranylcypromine, selegiline, haloperidol, pimozide, fluphenazine, thioridazine, chlorpromazine, trifluoperazine, molindone, loxapine, risperidone, olanzapine, quetiapine, ziprasidone, aripiprazole, clozapine, alprazolam, clonazepam, lorazepam, buspirone, zolpidem, eszopiclone, zaleplon, ramelteon, lithium, divalproex, valproate, carbamazepine, oxcarbazepine, lamotrigine, topiramate, acamprosate, buprenorphine, disulfiram, methadone, naltrexone, and gabapentin. In a still further aspect, the one or more compounds and the agent are administered sequentially. In yet a further aspect, the one or more compounds and the agent are administered simultaneously. In an even further aspect, the one or more compounds and the agent are co-formulated. In a still further aspect, the one or more compounds and the agent are not co-formulated.

In a further aspect, the agent known to treat a psychiatric disorder is a psychotropic. Examples of psychotropic agents include, but are not limited to, antidepressants (e.g., a tricyclic antidepressant, a selective serotonin reuptake inhibitor, a monoamine oxidase inhibitor), antipsychotics (e.g., haloperidol, loxapine, thioridazine, molindone, thiothixene, fluphenazine, mesoridazine, trifluoperazine), anxiolytics (e.g., a benzodiazepine, buspirone, hydroxyzine), mood stabilizers (e.g., valproic acid, lamotrigine, carbamazepine), medications for addiction (e.g., naltrexone, acamprosate, disulfiram), stimulants (e.g., methylphenidate, dextroamphetamine-amphetamine, dextroamphetamine), and psychedelics (e.g., ketamine, esketamine, dextromethorphan).

### C. METHODS FOR TREATING ANXIETY DISORDERS

In one aspect, In one aspect, disclosed are methods of treating an anxiety disorder in a subject in need thereof, the method comprising administering to the subject an effective amount of one or more compounds selected from pibrentasvir, glecaprevir, and velpatasvir, or pharmaceutically acceptable salts thereof.

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and new born subjects, as well as fetuses, whether male or female, are intended to be covered. The subject is preferably a mammal, such as a human. Prior to administering the compounds or compositions, the subject can be diagnosed with a need for treatment of an anxiety disorder, such as, for example, generalized anxiety disorder, panic disorder, social anxiety disorder, separation anxiety disorder, and agoraphobia.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, intrathecal administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically: that is, administered to treat an existing disease or condition. A preparation can also be administered prophylactically: that is, administered for prevention of an anxiety disorder, such as, for example, generalized anxiety disorder, panic disorder, social anxiety disorder, separation anxiety disorder, and agoraphobia.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

In a further aspect, the method comprises administering an individually effective amount of one or more compounds selected from pibrentasvir, glecaprevir, and velpatasvir, or pharmaceutically acceptable salts thereof. For example, in various aspects, the method comprises administering an individually effective amount of pibrentasvir or a pharmaceutically acceptable salt thereof. In a further aspect, the method comprises administering an individually effective amount of glecaprevir or a pharmaceutically acceptable salt thereof. In a still further aspect, the method comprises administering an individually effective amount of velpatasvir or a pharmaceutically acceptable salt thereof.

In a further aspect, the method comprises administering a synergistically effective amount of two or more compounds selected from pibrentasvir, glecaprevir, and velpatasvir, or pharmaceutically acceptable salts thereof. For example, in various aspects, the method comprises administering a synergistically effective amount of pibrentasvir and glecaprevir, or pharmaceutically acceptable salts thereof. In a further aspect, the method comprises administering a synergistically effective amount of pibrentasvir and velpatasivir, or pharmaceutically acceptable salts thereof. In a still further aspect, the method comprises administering a synergistically effective amount of glecaprevir and velpatasvir, or pharmaceutically acceptable salts thereof. In yet a further aspect, the method comprises administering a synergistically effective amount of pibrentasvir, glecaprevir, and velpatasvir, or pharmaceutically acceptable salts thereof.

In a further aspect, the method comprises administering exactly one of pibrentasvir, glecaprevir, and velpatasvir, or pharmaceutically acceptable salts thereof. For example, in various aspects, the method comprises administering pibrentasvir or a pharmaceutically acceptable salt thereof. In a further aspect, the method comprises administering glecaprevir or a pharmaceutically acceptable salt thereof. In a still further aspect, the method comprises administering velpatasvir or a pharmaceutically acceptable salt thereof.

In a further aspect, the method comprises administering two of pibrentasvir, glecaprevir, and velpatasvir, or pharmaceutically acceptable salts thereof. For example, in various aspects, the method comprises administering pibrentasvir and glecaprevir, or pharmaceutically acceptable salts thereof. In a further aspect, the method comprises administering pibrentasvir and velpatasvir, or pharmaceutically acceptable salts thereof. In a still further aspect, the method comprises administering glecaprevir and velpatasvir, or pharmaceutically acceptable salts thereof.

In a further aspect, the compounds are administered in a ratio of from about 1:10 to about 4:7, from about 1:8 to about 3:6, from about 1:7 to about 2:6, or from about 1:6 to about 1:3, or from about 1:6 to about 1:2. In a still further aspect, the compounds are administered in a ratio of about 1:10, about 1:5, about 3:10, about 2:5, about 1:2, about 3:5, about 7:10, about 4:5, or about 9:10. In yet a further aspect, the compounds are administered in a ratio of from about 4:7, about 3:7, about 2:7, or about 1:7. In an even further aspect, the compounds are administered in a ratio of about 2:5.

In a further aspect, the ratio of pibrentasvir to glecaprevir is of from about 1:10 to about 4:7, from about 1:8 to about 3:6, from about 1:7 to about 2:6, or from about 1:6 to about 1:3, or from about 1:6 to about 1:2. In a still further aspect, the ratio of pibrentasvir to glecaprevir is of about 1:10, about 1:5, about 3:10, about 2:5, about 1:2, about 3:5, about 7:10, about 4:5, or about 9:10. In yet a further aspect, the ratio of pibrentasvir to glecaprevir is of from about 4:7, about 3:7, about 2:7, or about 1:7. In an even further aspect, the ratio of pibrentasvir to glecaprevir is about 2:5.

In a further aspect, the ratio of glecaprevir to pibrentasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of glecaprevir to pibrentasvir is of about 1:10, about 1:5, about 3:10, about 2:5, about 1:2, about 3:5, about 7:10, about 4:5, or about 9:10. In yet a further aspect, the ratio of glecaprevir to pibrentasvir is of from about 4:7, about 3:7, about 2:7, or about 1:7. In an even further aspect, the ratio of glecaprevir to pibrentasvir is about 2:5.

In a further aspect, the ratio of pibrentasvir to velpatasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of pibrentasvir to velpatasvir is of about 1:10, about 1:5, about 3:10, about 2:5, about 1:2, about 3:5, about 7:10, about 4:5, or about 9:10. In yet a further aspect, the ratio of pibrentasvir to velpatasvir is of from about 4:7, about 3:7, about 2:7, or about 1:7. In an even further aspect, the ratio of pibrentasvir to velpatasvir is about 2:5.

In a further aspect, the ratio of velpatasvir to pibrentasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of velpatasvir to pibrentasvir is of about 1:10, about 1:5, about 3:10, about 2:5, about 1:2, about 3:5, about 7:10, about 4:5, or about 9:10. In yet a further aspect, the ratio of velpatasvir to pibrentasvir is of from about 4:7, about 3:7, about 2:7, or about 1:7. In an even further aspect, the ratio of velpatasvir to pibrentasvir is about 2:5.

In a further aspect, the ratio of velpatasvir to glecaprevir is of from about 1:10 to about 4:7, from about 1:8 to about 3:6, from about 1:7 to about 2:6, or from about 1:6 to about 1:3, or from about 1:6 to about 1:2. In a still further aspect, the ratio of velpatasvir to glecaprevir is of about 1:10, about 1:5, about 3:10, about 2:5, about 1:2, about 3:5, about 7:10, about 4:5, or about 9:10. In yet a further aspect, the ratio of velpatasvir to glecaprevir is of from about 4:7, about 3:7, about 2:7, or about 1:7. In an even further aspect, the ratio of velpatasvir to glecaprevir is about 2:5.

In a further aspect, the ratio of glecaprevir to velpatasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of glecaprevir to velpatasvir is of about 1:10, about 1:5, about 3:10, about 2:5, about 1:2, about 3:5, about 7:10, about 4:5, or about 9:10. In yet a further aspect, the ratio of glecaprevir to velpatasvir is of from about 4:7, about 3:7, about 2:7, or about 1:7. In an even further aspect, the ratio of glecaprevir to velpatasvir is about 2:5.

In a further aspect, the method comprises administering pibrentasvir, glecaprevir, and velpatasvir, or pharmaceutically acceptable salts thereof.

In a further aspect, when more than one compound is administered, the compounds can be administered sequentially. For example, in various aspects, pibrentasvir and glecaprevir are administered sequentially. In a further aspect, pibrentasvir and velpatasvir are administered sequentially. In a still further aspect, glecaprevir and velpatasvir are administered sequentially.

In a further aspect, when more than one compound is administered, the compounds can be administered simultaneously. For example, in various aspects, pibrentasvir and glecaprevir are administered simultaneously. In a further aspect, pibrentasvir and velpatasvir are administered simultaneously. In a still further aspect, glecaprevir and velpatasvir are administered simultaneously.

In a further aspect, when more than one compound is administered, the compounds can be co-formulated. For example, in various aspects, pibrentasvir and glecaprevir are co-formulated. In a further aspect, pibrentasvir and velpatasvir are co-formulated. In a still further aspect, glecaprevir and velpatasvir are co-formulated.

In various further aspects, the compounds can be co-formulated as an oral dosage form such as, for example, a tablet or oral pellets. For example, in various aspects, pibrentasvir and glecaprevir are co-formulated as an oral dosage form. In a further aspect, pibrentasvir and velpatasvir are co-formulated as an oral dosage form. In a still further aspect, glecaprevir and velpatasvir are co-formulated as an oral dosage form.

In various further aspects, the compounds can be co-formulated as a single oral dosage form. For example, in various aspects, pibrentasvir and glecaprevir are co-formulated as a single oral dosage form. In a further aspect, pibrentasvir and velpatasvir are co-formulated as a single oral dosage form. In a still further aspect, glecaprevir and velpatasvir are co-formulated as a single oral dosage form.

In a further aspect, the one or more compounds are administered via oral, sublingual, topical, rectal, transdermal, injection (e.g., intravenous, intramuscular), or nasal administration. In a still further aspect, the one or more compounds are administered via oral administration.

In a further aspect, the method is for treating an anxiety disorder. Examples of anxiety disorders include, but are not limited to, generalized anxiety disorder, panic disorder, social anxiety disorder, separation anxiety disorder, and agoraphobia.

In a further aspect, the subject has not been diagnosed as having hepatitis C virus. In a still further aspect, the subject has not been diagnosed as having hepatitis C virus within the last 7 days. In yet a further aspect, the subject has not been diagnosed as having hepatitis C virus within the last 14 days. In an even further aspect, the subject has not been diagnosed as having hepatitis C virus within the last month. In a still further aspect, the subject has not been diagnosed as having hepatitis C virus within the last six months.

In a further aspect, the subject has not been diagnosed as having an infection due to a flavivirus. In a still further aspect, the subject has not been diagnosed as having an infection due to a flavivirus within the last 7 days. In yet a further aspect, the subject has not been diagnosed as having an infection due to a flavivirus within the last 14 days. In an even further aspect, the subject has not been diagnosed as having an infection due to a flavivirus within the last month. In a still further aspect, the subject has not been diagnosed as having an infection due to a flavivirus within the last six months.

In a further aspect, the subject is not currently undergoing treatment for hepatitis C virus. In a still further aspect, the subject has not previously undergone treatment for hepatitis C virus within the last 7 days. In yet a further aspect, the subject has not previously undergone treatment for hepatitis C virus within the last 14 days. In an even further aspect, the subject has not previously undergone treatment for hepatitis C virus within the last month. In a still further aspect, the subject has not previously undergone treatment for hepatitis C virus within the last six months.

In a further aspect, the subject is not currently undergoing treatment for an infection due to a flavivirus. In a still further aspect, the subject has not previously undergone treatment for an infection due to a flavivirus within the last 7 days. In yet a further aspect, the subject has not previously undergone treatment for an infection due to a flavivirus within the last 14 days. In an even further aspect, the subject has not previously undergone treatment for an infection due to a flavivirus within the last month. In a still further aspect, the subject has not previously undergone treatment for an infection due to a flavivirus within the last six months.

In a further aspect, the subject is a mammal. In a still further aspect, the mammal is a human. In yet a further aspect, the human is an adult (e.g., 18 years of age or older). In a still further aspect, the human is a child (e.g., 3-17 years of age).

In a further aspect, the subject has been diagnosed with a need for treatment of the anxiety disorder prior to the administering step. In a still further aspect, the method further comprises the step of identifying a subject in need of treatment of an anxiety disorder.

In a further aspect, the effective amount is a therapeutically effective amount. In a still further aspect, the effective amount is a prophylactically effective amount.

In a further aspect, the effective amount is an amount of from about 0.1 mg/kg to about 0.4 mg/kg, about 0.1 mg/kg to about 0.3 mg/kg, about 0.1 mg/kg to about 0.2 mg/kg, about 0.2 mg/kg to about 0.4 mg/kg, about 0.3 mg/kg to about 0.4 mg/kg, or about 0.2 mg/kg to about 0.3 mg/kg. In a still further aspect, the effective amount is an amount of about 0.1 mg/kg, about 0.15 mg/kg, about 0.2 mg/kg, about 0.25 mg/kg, about 0.3 mg/kg, about 0.35 mg/kg, or about 0.4 mg/kg. In yet a further aspect, the effective amount is an amount of about 0.25 mg/kg.

In a further aspect, the effective amount is administered once per day. In a still further aspect, the effective amount is administered once per day for a period of at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days. In yet a further aspect, the effective amount is administered once per day for a period of at least 7 days. In an even further aspect, the effective amount is administered once per day for a period of at least 14 days.

In a further aspect, administering is via systemic administration.

In a further aspect, the method further comprises administering to the subject an effective amount of an agent known to treat an anxiety disorder. Examples of agents known to treat anxiety disorders include, but are not limited to, selective serotonin reuptake inhibitors (e.g., citalopram, escitalopram oxalate, fluoxetine, fluvoxamine, paroxetine HRI, sertraline), selective serotonin and norepinephrine inhibitors (e.g., desvenlafaxine, desvenlafaxine succinate, duloxetine, levomilnacipran, venlafaxine), vortioxetine, vilazodone, a tetracyclic antidepressant and specific serotonergic antidepressant (e.g., mirtazapine), tricyclic antidepressants (e.g., amitriptyline, imipramine, nortriptyline, doxepin), bupropion, monoamine oxidase inhibitors (e.g., isocarboxazid, phenelzine, selegine, tranylcypromine), N-methyl-D-aspartate receptor antagonists (e.g., ketamine, esketamine), and 1-methylfolate. In a still further aspect, the one or more compounds and the agent are administered sequentially. In yet a further aspect, the one or more compounds and the agent are administered simultaneously. In an even further aspect, the one or more compounds and the agent are co-formulated. In a still further aspect, the one or more compounds and the agent are not co-formulated.

### D. METHODS FOR TREATING A SUBSTANCE-USE DISORDER

In one aspect, disclosed are methods of treating a substance-use disorder in a subject in need thereof, the method comprising administering to the subject an effective amount of one or more compounds selected from pibrentasvir, glecaprevir, velpatasvir, ledipasvir, and sofosbuvir, or pharmaceutically acceptable salts thereof, wherein the subject has not been diagnosed as having hepatitis C virus.

In a further aspect, the method comprises administering an individually effective amount of one or more compounds selected from pibrentasvir, glecaprevir, velpatasvir, ledipasvir, and sofosbuvir, or pharmaceutically acceptable salts thereof. In a still further aspect, the method comprises administering a synergistically effective amount of two or more compounds selected from pibrentasvir, glecaprevir, velpatasvir, ledipasvir, and sofosbuvir, or pharmaceutically acceptable salts thereof.

In a further aspect, the method comprises administering exactly one of pibrentasvir, glecaprevir, velpatasvir, ledipasvir, and sofosbuvir, or pharmaceutically acceptable salts thereof. In a still further aspect, the method comprises administering two of pibrentasvir, glecaprevir, velpatasvir, ledipasvir, and sofosbuvir, or pharmaceutically acceptable salts thereof.

In a further aspect, the method comprises administering pibrentasvir or a pharmaceutically acceptable salt thereof. In a still further aspect, the method comprises administering glecaprevir or a pharmaceutically acceptable salt thereof. In yet a further aspect, the method comprises administering velpatasvir or a pharmaceutically acceptable salt thereof. In an even further aspect, the method comprises administering ledipasvir or a pharmaceutically acceptable salt thereof. In a still further aspect, the method comprises administering sofosbuvir or a pharmaceutically acceptable salt thereof.

In a further aspect, the method comprises administering pibrentasvir and glecaprevir. In a still further aspect, pibrentasvir and glecaprevir are administered sequentially. In yet a further aspect, pibrentasvir and glecaprevir are administered simultaneously.

In a further aspect, pibrentasvir and glecaprevir are co-formulated. In a still further aspect, the ratio of pibrentasvir to glecaprevir is of from about 1:10 to about 4:7, from about 1:8 to about 3:6, from about 1:7 to about 2:6, or from about 1:6 to about 1:3, or from about 1:6 to about 1:2. In a still further aspect, the ratio of pibrentasvir to glecaprevir is of about 1:10, about 1:5, about 3:10, about 2:5, about 1:2, about 3:5, about 7:10, about 4:5, or about 9:10. In yet a further aspect, the ratio of pibrentasvir to glecaprevir is of from about 4:7, about 3:7, about 2:7, or about 1:7. In an even further aspect, the ratio of pibrentasvir to glecaprevir is about 2:5.

In a further aspect, pibrentasvir and glecaprevir are co-formulated as an oral dosage form. In a still further aspect, the oral dosage form is a tablet or oral pellets. In yet a further aspect, pibrentasvir and glecaprevir are co-formulated as a single oral dosage form.

In a further aspect, the method comprises administering velpatasvir and sofosbuvir. In a still further aspect, velpatasvir and sofosbuvir are administered sequentially. In yet a further aspect, velpatasvir and sofosbuvir are administered simultaneously.

In a further aspect, velpatasvir and sofosbuvir are co-formulated. In a still further aspect, the ratio of velpatasvir to sofosbuvir is of from about 1:16 to about 1:3, about 1:14 to about 1:3, about 1:12 to about 1:3, about 1:10 to about 1:3, about 1:8 to about 1:3, about 1:6 to about 1:3, about 1:4 to about 1:3, about 1:16 to about 1:4, about 1:16 to about 1:6, about 1:16 to about 1:6, about 1:16 to about 1:8, about 1:16 to about 1:10, about 1:16 to about 1:12, about 1:16 to about 1:14, about 1:14 to about 1:4, about 1:12 to about 1:6, or about 1:10 to about 1:8. In yet a further aspect, the ratio of velpatasvir to sofosbuvir is about 1:4.

In a further aspect, velpatasvir and sofosbuvir are co-formulated as an oral dosage form. In a still further aspect, the oral dosage form is a tablet or oral pellets. In yet a further aspect, velpatasvir and sofosbuvir are co-formulated as a single oral dosage form.

In a further aspect, the method comprises administering ledipasvir and sofosbuvir. In a further aspect, ledipasvir and sofosbuvir are administered sequentially. In a still further aspect, ledipasvir and sofosbuvir are administered simultaneously:

In a further aspect, ledipasvir and sofosbuvir are co-formulated. In a still further aspect, the ratio of ledipasvir to sofosbuvir is of from about 1:20 to about 3:10, about 1:15 to about 3:10, about 1:10 to about 3:10 about 1:5 to about 3:10, about 1:20 to about 2:5, about 1:20 to about 1:2, about 1:15 to about 2:5, or about 1:10 to about 1:2. In yet a further aspect, the ratio of ledipasvir to sofosbuvir is about 9:40.

In a further aspect, ledipasvir and sofosbuvir are co-formulated as an oral dosage form. In a still further aspect, the oral dosage form is a tablet or oral pellets. In yet a further aspect, ledipasvir and sofosbuvir are co-formulated as a single oral dosage form.

In a further aspect, the one or more compounds are administered orally.

In a further aspect, the substance-use disorder is alcohol use disorder.

In a further aspect, the subject is a human.

In a further aspect, the subject is not currently undergoing treatment for hepatitis C infection. In a still further aspect, the subject is not currently undergoing treatment for an infection due to a flavivirus.

In a further aspect, the subject has been diagnosed with a need for treatment of posttraumatic stress disorder prior to the administering step. In a still further aspect, the subject has been diagnosed with a need for treatment of the substance-use disorder prior to the administering step.

In a further aspect, the method further comprises identifying a subject in need of treatment of the substance-use disorder.

In a further aspect, the effective amount is a therapeutically effective amount. In a still further aspect, the effective amount is a prophylactically effective amount.

### E. PHARMACEUTICAL COMPOSITIONS

In one aspect, disclosed are pharmaceutical compositions comprising an effective amount of one or more compounds selected from pibrentasvir, glecaprevir, and velpatasvir, or pharmaceutically acceptable salts thereof, and an effective amount of an agent known to treat a psychiatric disorder, and a pharmaceutically acceptable carrier.

In one aspect, disclosed are pharmaceutical compositions comprising an effective amount of one or more compounds selected from pibrentasvir, glecaprevir, velpatasvir, ledipasvir, and sofosbuvir, or pharmaceutically acceptable salts thereof, and an effective amount of an agent known to treat a psychiatric disorder, and a pharmaceutically acceptable carrier.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457.

The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In various aspects, the disclosed pharmaceutical compositions comprise the disclosed compounds (including pharmaceutically acceptable salt(s) thereof) as an active ingredient, a pharmaceutically acceptable carrier, and, optionally; other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage: thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions: while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, the composition comprises an individually effective amount of one or more compounds selected from pibrentasvir, glecaprevir, and velpatasvir, or pharmaceutically acceptable salts thereof. For example, in various aspects, the composition comprises an individually effective amount of pibrentasvir or a pharmaceutically acceptable salt thereof. In a further aspect, the composition comprises an individually effective amount of glecaprevir or a pharmaceutically acceptable salt thereof. In a still further aspect, the composition comprises an individually effective amount of velpatasvir or a pharmaceutically acceptable salt thereof.

In a further aspect, the composition comprises an individually effective amount of one or more compounds selected from pibrentasvir, glecaprevir, velpatasvir, ledipasvir, and sofosbuvir, or pharmaceutically acceptable salts thereof. For example, in various aspects, the composition comprises an individually effective amount of pibrentasvir or a pharmaceutically acceptable salt thereof. In a further aspect, the composition comprises an individually effective amount of glecaprevir or a pharmaceutically acceptable salt thereof. In a still further aspect, the composition comprises an individually effective amount of velpatasvir or a pharmaceutically acceptable salt thereof. In yet a further aspect, the composition comprises an individually effective amount of ledipasvir or a pharmaceutically acceptable salt thereof. In an even further aspect, the composition comprises an individually effective amount of sofosbuvir or a pharmaceutically acceptable salt thereof.

In a further aspect, the composition comprises a synergistically effective amount of two or more compounds selected from pibrentasvir, glecaprevir, and velpatasvir, or pharmaceutically acceptable salts thereof. For example, in various aspects, the composition comprises a synergistically effective amount of pibrentasvir and glecaprevir, or pharmaceutically acceptable salts thereof. In a further aspect, the composition comprises a synergistically effective amount of pibrentasvir and velpatasivir, or pharmaceutically acceptable salts thereof. In a still further aspect, the composition comprises a synergistically effective amount of glecaprevir and velpatasvir, or pharmaceutically acceptable salts thereof. In yet a further aspect, the composition comprises a synergistically effective amount of pibrentasvir, glecaprevir, and velpatasvir, or pharmaceutically acceptable salts thereof.

In a further aspect, the composition comprises a synergistically effective amount of two or more compounds selected from pibrentasvir, glecaprevir, velpatasvir, ledipasvir, and sofosbuvir, or pharmaceutically acceptable salts thereof. For example, in various aspects, the composition comprises a synergistically effective amount of pibrentasvir and glecaprevir, or pharmaceutically acceptable salts thereof. In a further aspect, the composition comprises a synergistically effective amount of pibrentasvir and velpatasivir, or pharmaceutically acceptable salts thereof. In a still further aspect, the composition comprises a synergistically effective amount of glecaprevir and velpatasvir, or pharmaceutically acceptable salts thereof. In yet a further aspect, the composition comprises a synergistically effective amount of glecaprevir and ledipasvir, or pharmaceutically acceptable salts thereof. In an even further aspect, the composition comprises a synergistically effective amount of glecaprevir and sofosbuvir, or pharmaceutically acceptable salts thereof. In a still further aspect, the composition comprises a synergistically effective amount of pibrentasvir and ledipasvir, or pharmaceutically acceptable salts thereof. In yet a further aspect, the composition comprises a synergistically effective amount of pibrentasvir and sofosbuvir, or pharmaceutically acceptable salts thereof. In an even further aspect, the composition comprises a synergistically effective amount of velpatasvir and ledipasvir, or pharmaceutically acceptable salts thereof. In a still further aspect, the composition comprises a synergistically effective amount of velpatasvir and sofosbuvir, or pharmaceutically acceptable salts thereof. In yet a further aspect, the composition comprises a synergistically effective amount of sofosbuvir and sofosbuvir, or pharmaceutically acceptable salts thereof. In an even further aspect, the composition comprises a synergistically effective amount of pibrentasvir, glecaprevir, and velpatasvir, or pharmaceutically acceptable salts thereof. In a still further aspect, the composition comprises a synergistically effective amount of pibrentasvir, glecaprevir, and ledipasvir, or pharmaceutically acceptable salts thereof. In yet a further aspect, the composition comprises a synergistically effective amount of pibrentasvir, ledipasvir, and velpatasvir, or pharmaceutically acceptable salts thereof. In an even further aspect, the composition comprises a synergistically effective amount of ledipasvir, glecaprevir, and velpatasvir, or pharmaceutically acceptable salts thereof. In a still further aspect, the composition comprises a synergistically effective amount of pibrentasvir, glecaprevir, and sofosbuvir, or pharmaceutically acceptable salts thereof. In yet a further aspect, the composition comprises a synergistically effective amount of pibrentasvir, sofosbuvir, and velpatasvir, or pharmaceutically acceptable salts thereof. In an even further aspect, the composition comprises a synergistically effective amount of sofosbuvir, glecaprevir, and velpatasvir, or pharmaceutically acceptable salts thereof. In a still further aspect, the composition comprises a synergistically effective amount of pibrentasvir, ledipasvir, and sofosbuvir, or pharmaceutically acceptable salts thereof. In yet a further aspect, the composition comprises a synergistically effective amount of ledipasvir, sofosbuvir, and velpatasvir, or pharmaceutically acceptable salts thereof. In an even further aspect, the composition comprises a synergistically effective amount of sofosbuvir, glecaprevir, and ledipasvir, or pharmaceutically acceptable salts thereof. In a still further aspect, the composition comprises a synergistically effective amount of sofosbuvir, glecaprevir, ledipasvir, and pibrentasvir, or pharmaceutically acceptable salts thereof.

In a further aspect, the composition comprises exactly one of pibrentasvir, glecaprevir, and velpatasvir, or pharmaceutically acceptable salts thereof. For example, in various aspects, the composition comprises pibrentasvir or a pharmaceutically acceptable salt thereof. In a further aspect, the composition comprises glecaprevir or a pharmaceutically acceptable salt thereof. In a still further aspect, the composition comprises velpatasvir or a pharmaceutically acceptable salt thereof.

In a further aspect, the composition comprises exactly one of pibrentasvir, glecaprevir, velpatasvir, ledipasvir, and sofosbuvir, or pharmaceutically acceptable salts thereof. For example, in various aspects, the composition comprises pibrentasvir or a pharmaceutically acceptable salt thereof. In a further aspect, the composition comprises glecaprevir or a pharmaceutically acceptable salt thereof. In a still further aspect, the composition comprises velpatasvir or a pharmaceutically acceptable salt thereof. In yet a further aspect, the composition comprises ledipasvir or a pharmaceutically acceptable salt thereof. In an even further aspect, the composition comprises sofosbuvir or a pharmaceutically acceptable salt thereof.

In a further aspect, the composition comprises exactly two of pibrentasvir, glecaprevir, and velpatasvir, or pharmaceutically acceptable salts thereof. For example, in various aspects, the composition comprises pibrentasvir and glecaprevir, or pharmaceutically acceptable salts thereof. In a further aspect, the composition comprises pibrentasvir and velpatasvir, or pharmaceutically acceptable salts thereof. In a still further aspect, the composition comprises glecaprevir and velpatasvir, or pharmaceutically acceptable salts thereof.

In a further aspect, the composition comprises exactly two of pibrentasvir, glecaprevir, velpatasvir, ledipasvir, and sofosbuvir, or pharmaceutically acceptable salts thereof. For example, in various aspects, the composition comprises pibrentasvir and glecaprevir, or pharmaceutically acceptable salts thereof. In a further aspect, the composition comprises pibrentasvir and velpatasvir, or pharmaceutically acceptable salts thereof. In a still further aspect, the composition comprises glecaprevir and velpatasvir, or pharmaceutically acceptable salts thereof. In yet a further aspect, the composition comprises glecaprevir and ledipasvir, or pharmaceutically acceptable salts thereof. In an even further aspect, the composition comprises ledipasvir and velpatasvir, or pharmaceutically acceptable salts thereof. In a still further aspect, the composition comprises pibrentasvir and ledipasvir, or pharmaceutically acceptable salts thereof. In yet a further aspect, the composition comprises ledipasvir and sofosbuvir, or pharmaceutically acceptable salts thereof. In an even further aspect, the composition comprises pibrentasvir and sofosbuvir, or pharmaceutically acceptable salts thereof. In a still further aspect, the composition comprises glecaprevir and sofosbuvir, or pharmaceutically acceptable salts thereof. In yet a further aspect, the composition comprises velpatasvir and sofosbuvir, or pharmaceutically acceptable salts thereof.

In a further aspect, exactly two of the compounds are present. Thus, in various aspects, the two compounds are present in a ratio of from about 1:10 to about 4:7, from about 1:8 to about 3:6, from about 1:7 to about 2:6, or from about 1:6 to about 1:3, or from about 1:6 to about 1:2. In a still further aspect, the two compounds are present in a ratio of about 1:10, about 1:5, about 3:10, about 2:5, about 1:2, about 3:5, about 7:10, about 4:5, or about 9:10. In yet a further aspect, the two compounds are present in a ratio of from about 4:7, about 3:7, about 2:7, or about 1:7. In an even further aspect, the two compounds are present in a ratio of about 2:5.

In a further aspect, the composition comprises pibrentasvir and glecaprevir. Thus, in various aspects, the ratio of pibrentasvir to glecaprevir is of from about 1:10 to about 4:7, from about 1:8 to about 3:6, from about 1:7 to about 2:6, or from about 1:6 to about 1:3, or from about 1:6 to about 1:2. In a still further aspect, the ratio of pibrentasvir to glecaprevir is of about 1:10, about 1:5, about 3:10, about 2:5, about 1:2, about 3:5, about 7:10, about 4:5, or about 9:10. In yet a further aspect, the ratio of pibrentasvir to glecaprevir is of from about 4:7, about 3:7, about 2:7, or about 1:7. In an even further aspect, the ratio of pibrentasvir to glecaprevir is about 2:5.

In a further aspect, the ratio of glecaprevir to pibrentasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of glecaprevir to pibrentasvir is of about 1:10, about 1:5, about 3:10, about 2:5, about 1:2, about 3:5, about 7:10, about 4:5, or about 9:10. In yet a further aspect, the ratio of glecaprevir to pibrentasvir is of from about 4:7, about 3:7, about 2:7, or about 1:7. In an even further aspect, the ratio of glecaprevir to pibrentasvir is about 2:5.

In a further aspect, the composition comprises pibrentasvir and velpatasvir. Thus, in various aspects, the ratio of pibrentasvir to velpatasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of pibrentasvir to velpatasvir is of about 1:10, about 1:5, about 3:10, about 2:5, about 1:2, about 3:5, about 7:10, about 4:5, or about 9:10. In yet a further aspect, the ratio of pibrentasvir to velpatasvir is of from about 4:7, about 3:7, about 2:7, or about 1:7. In an even further aspect, the ratio of pibrentasvir to velpatasvir is about 2:5.

In a further aspect, the ratio of velpatasvir to pibrentasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of velpatasvir to pibrentasvir is of about 1:10, about 1:5, about 3:10, about 2:5, about 1:2, about 3:5, about 7:10, about 4:5, or about 9:10. In yet a further aspect, the ratio of velpatasvir to pibrentasvir is of from about 4:7, about 3:7, about 2:7, or about 1:7. In an even further aspect, the ratio of velpatasvir to pibrentasvir is about 2:5.

In a further aspect, the composition comprises velpatasvir and glecaprevir. Thus, in various aspects, the ratio of velpatasvir to glecaprevir is of from about 1:10 to about 4:7, from about 1:8 to about 3:6, from about 1:7 to about 2:6, or from about 1:6 to about 1:3, or from about 1:6 to about 1:2. In a still further aspect, the ratio of velpatasvir to glecaprevir is of about 1:10, about 1:5, about 3:10, about 2:5, about 1:2, about 3:5, about 7:10, about 4:5, or about 9:10. In yet a further aspect, the ratio of velpatasvir to glecaprevir is of from about 4:7, about 3:7, about 2:7, or about 1:7. In an even further aspect, the ratio of velpatasvir to glecaprevir is about 2:5.

In a further aspect, the ratio of glecaprevir to velpatasvir is of from about 1:10 to about 4:7, from about 1:8 to about 3:6, from about 1:7 to about 2:6, or from about 1:6 to about 1:3, or from about 1:6 to about 1:2. In a still further aspect, the ratio of glecaprevir to velpatasvir is of about 1:10, about 1:5, about 3:10, about 2:5, about 1:2, about 3:5, about 7:10, about 4:5, or about 9:10. In yet a further aspect, the ratio of glecaprevir to velpatasvir is of from about 4:7, about 3:7, about 2:7, or about 1:7. In an even further aspect, the ratio of glecaprevir to velpatasvir is about 2:5.

In a further aspect, the composition comprises velpatasvir and sofosbuvir. In a further aspect, the ratio of velpatasvir to sofosbuvir is of from about 1:16 to about 1:3, from about 1:12 to about 1:5, or about 1:10 to about 1:7. In a still further aspect, the ratio of velpatasvir to sofosbuvir is about 1:4.

In a further aspect, the composition comprises ledipasvir to sofosbuvir. In a further aspect, the ratio of ledipasvir to sofosbuvir is of from about 1:20 to about 3:10, about 1:15 to about 3:10, or about 1:10 to about 3:10. In a still further aspect, the ratio of ledipasvir to sofosbuvir is about 9:40.

In a further aspect, the composition comprises each of pibrentasvir, glecaprevir, and velpatasvir, or pharmaceutically acceptable salts thereof.

In a further aspect, the effective amount is a therapeutically effective amount. In a still further aspect, the effective amount is a prophylactically effective amount.

In a further aspect, the effective amount is an amount of from about 0.1 mg/kg to about 0.4 mg/kg, about 0.1 mg/kg to about 0.3 mg/kg, about 0.1 mg/kg to about 0.2 mg/kg, about 0.2 mg/kg to about 0.4 mg/kg, about 0.3 mg/kg to about 0.4 mg/kg, or about 0.2 mg/kg to about 0.3 mg/kg. In a still further aspect, the effective amount is an amount of about 0.1 mg/kg, about 0.15 mg/kg, about 0.2 mg/kg, about 0.25 mg/kg, about 0.3 mg/kg, about 0.35 mg/kg, or about 0.4 mg/kg. In yet a further aspect, the effective amount is an amount of about 0.25 mg/kg.

In a further aspect, the composition comprises an effective amount of the agent known to treat a psychiatric disorder. Examples of agents known to treat psychiatric disorders include, but are not limited to, methylphenidate, amphetamine, atomoxetine, guanfacine, clonidine, fluoxetine, sertraline, fluvoxamine, paroxetine, citalopram, escitalopram, imipramine, desipramine, amitryptyline, nortriptyline, doxepin, clomipramine, bupropion, venlafaxine, mirtazapine, duloxetine, phenelzine, tranylcypromine, selegiline, haloperidol, pimozide, fluphenazine, thioridazine, chlorpromazine, trifluoperazine, molindone, loxapine, risperidone, olanzapine, quetiapine, ziprasidone, aripiprazole, clozapine, alprazolam, clonazepam, lorazepam, buspirone, zolpidem, eszopiclone, zaleplon, ramelteon, lithium, divalproex, valproate, carbamazepine, oxcarbazepine, lamotrigine, topiramate, acamprosate, buprenorphine, disulfiram, methadone, naltrexone, and gabapentin. In a still further aspect, the agent known to treat a psychiatric disorder is a psychotropic. In yet a further aspect, the psychotropic is selected from an antidepressant (e.g., a tricyclic antidepressant, a selective serotonin reuptake inhibitor, a monoamine oxidase inhibitor), an antipsychotic (e.g., haloperidol, loxapine, thioridazine, molindone, thiothixene, fluphenazine, mesoridazine, trifluoperazine), an anxiolytic (e.g., a benzodiazepine, buspirone, hydroxyzine), a mood stabilizer (e.g., valproic acid, lamotrigine, carbamazepine), a medication for addiction (e.g., naltrexone, acamprosate, disulfiram), a stimulant (e.g., methylphenidate, dextroamphetamine-amphetamine, dextroamphetamine), and a psychedelic (e.g., ketamine, esketamine, dextromethorphan).

It is understood that the disclosed compositions can be prepared from the disclosed compounds. It is also understood that the disclosed compositions can be employed in the disclosed methods of using.

### F. KITS

In one aspect, disclosed are kits comprising one or more compounds selected from pibrentasvir, glecaprevir, and velpatasvir, or pharmaceutically acceptable salts thereof, and one or more of: (a) an agent known to treat a psychiatric disorder: (b) instructions for treating a psychiatric disorder; and (c) instructions for administering the one or more compounds in connection with treating a psychiatric disorder.

In one aspect, disclosed are kits comprising one or more compounds selected from pibrentasvir, glecaprevir, velpatasvir, ledipasvir, and sofosbuvir, or pharmaceutically acceptable salts thereof, and one or more of: (a) an agent known to treat a psychiatric disorder: (b) instructions for treating a psychiatric disorder; and (c) instructions for administering the one or more compounds in connection with treating a psychiatric disorder.

In a further aspect, the kit comprises pibrentasvir or a pharmaceutically acceptable salt thereof. In a still further aspect, the kit comprises glecaprevir or a pharmaceutically acceptable salt thereof. In yet a further aspect, the kit comprises velpatasvir or a pharmaceutically acceptable salt thereof.

In a further aspect, the kit comprises pibrentasvir and glecaprevir, or pharmaceutically acceptable salts thereof. In a still further aspect, the kit comprises pibrentasvir and velpatasvir, or pharmaceutically acceptable salts thereof. In yet a further aspect, the kit comprises glecaprevir and velpatasvir, or pharmaceutically acceptable salts thereof. In an even further aspect, the kit comprises ledipasvir and sofosbuvir, or pharmaceutically acceptable salts thereof. In a still further aspect, the kit comprises velpatasvir and sofosbuvir, or pharmaceutically acceptable salts thereof.

In a further aspect, the kit comprises the agent known to treat a psychiatric disorder. Examples of agents known to treat psychiatric disorders include, but are not limited to, methylphenidate, amphetamine, atomoxetine, guanfacine, clonidine, fluoxetine, sertraline, fluvoxamine, paroxetine, citalopram, escitalopram, imipramine, desipramine, amitryptyline, nortriptyline, doxepin, clomipramine, bupropion, venlafaxine, mirtazapine, duloxetine, phenelzine, tranylcypromine, selegiline, haloperidol, pimozide, fluphenazine, thioridazine, chlorpromazine, trifluoperazine, molindone, loxapine, risperidone, olanzapine, quetiapine, ziprasidone, aripiprazole, clozapine, alprazolam, clonazepam, lorazepam, buspirone, zolpidem, eszopiclone, zaleplon, ramelteon, lithium, divalproex, valproate, carbamazepine, oxcarbazepine, lamotrigine, topiramate, acamprosate, buprenorphine, disulfiram, methadone, naltrexone, and gabapentin. In a further aspect, the agent known to treat a psychiatric disorder is a psychotropic. Examples of psychotropic agents include, but are not limited to, antidepressants (e.g., a tricyclic antidepressant, a selective serotonin reuptake inhibitor, a monoamine oxidase inhibitor), antipsychotics (e.g., haloperidol, loxapine, thioridazine, molindone, thiothixene, fluphenazine, mesoridazine, trifluoperazine), anxiolytics (e.g., a benzodiazepine, buspirone, hydroxyzine), mood stabilizers (e.g., valproic acid, lamotrigine, carbamazepine), medications for addiction (e.g., naltrexone, acamprosate, disulfiram), stimulants (e.g., methylphenidate, dextroamphetamine-amphetamine, dextroamphetamine), and psychedelics (e.g., ketamine, esketamine, dextromethorphan). In a still further aspect, the one or more compounds and the agent known to treat a psychiatric disorder are co-packaged. In yet a further aspect, the one or more compounds and the agent known to treat a psychiatric disorder are co-formulated.

As would be understood by one of ordinary skill in the art, examples of psychiatric disorders include, but are not limited to, neurodevelopmental disorders (e.g., attention deficit/hyperactivity disorder, autism spectrum disorder, cerebral palsy), schizophrenia spectrum disorders (e.g., schizophrenia, schizoaffective disorder, delusional disorder, schizoptypal personality disorder, schizophreniform disorder), depressive disorders (e.g., bipolar disorder, major depression, persistent depressive disorder, seasonal affective disorder, psychotic depression, peripartum depression, treatment resistant depression, atypical depression, premenstrual dysphoric disorder), anxiety disorders (e.g., generalized anxiety disorder, panic disorder, social anxiety disorder, separation anxiety disorder, agoraphobia), obsessive-compulsive disorder, trauma- or stressor-related disorders (posttraumatic stress disorder, acute stress disorder, an adjustment disorder, reactive attachment disorder, disinhibited social engagement disorder), dissociative disorders (e.g., dissociative amnesia, dissociative fugue, depersonalization disorder, dissociative identity disorder), somatic symptom disorder, feeding or eating disorders (e.g., bulimia nervosa, anorexia nervosa, avoidant/restrictive food intake disorder), elimination disorders (e.g., encopresis, enuresisis), sleep-wake disorders (e.g., insomnia, obstructive sleep apnea, parasomnia, narcolepsy, restless leg syndrome), sexual dysfunctions or disorders (e.g., a desire disorder, an arousal disorder, an orgasm disorder, a pain disorder), gender dysphoria, disruptive, impulse-control, and conduct disorders (e.g., oppositional defiant disorder, intermittent explosive disorder, conduct disorder, pyromania, kleptomania), substance-related and addictive disorders (e.g., a substance-induced disorder, a substance-use disorder (e.g., an alcohol use disorder), substance-induced psychosis, withdrawal, substance-induced delirium), neurocognitive disorders (e.g., Alzheimer's disease, vascular dementia, dementia with Lewy body, frontotemporal lobar degeneration), personality disorders (e.g., borderline personality disorder, antisocial personality disorder, histrionic personality disorder, narcissistic personality disorder, obsessive-compulsive personality disorder, schizoid personality disorder, schizotypal personality disorder), and paraphilic disorders (e.g., exhibitionistic disorder, fetishistic disorder, frotteuristic disorder, pedophilic disorder, sexual masochism disorder, sexual sadism disorder, transvestic disorder, voyeuristic disorder).

In a further aspect, the psychiatric disorder is a trauma- or stressor-related disorder. In a still further aspect, the trauma- or stressor-related disorder is selected form posttraumatic stress disorder, acute stress disorder, an adjustment disorder, reactive attachment disorder, and disinhibited social engagement disorder. In yet a further aspect, the trauma- or stressor-related disorder is posttraumatic stress disorder.

In a further aspect, the psychiatric disorder is a substance-use disorder. In a still further aspect, the substance-use disorder is alcohol use disorder.

In a further aspect, the psychiatric disorder is a co-morbid disorder. In a still further aspect, the co-morbid disorder is posttraumatic stress disorder and alcohol use disorder.

It is understood that the disclosed kits can be prepared from the disclosed compounds, products, and pharmaceutical compositions. It is also understood that the disclosed kits can be employed in connection with the disclosed methods of using.

### G. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Treescan for PTSD Medications

Despite the high prevalence and negative impact of Posttraumatic Stress Disorder (PTSD: e.g., Gradus et al. (2015) Am J Epidemiol. 182:451-458: Hoge et al. (2007) Am J Psychiatry. 164:150-153; Pietrzak (2011) J Anxiety Disord. 25:456-465), there are few effective pharmacologic treatments for PTSD and even these effective medications do not work well enough (Shiner et al. (2020) J Clin Psychiatry. 81: Shiner et al. (2018) J Clin Psychiatry. 79). In 2017, the Veterans Affairs (VA) PTSD Psychopharmacology Work Group published a statement in Biological Psychiatry describing key issues that are leading to a PTSD “pharmacotherapy crisis” (Krystal et al. (2017) Biol Psychiatry. 82: e51-e59). First, antidepressants, the most prescribed pharmacologic agents for PTSD, are associated with remission in fewer than half of the patients who take them. Currently, only sertraline and paroxetine are approved for the treatment of PTSD by the US Food and Drug Administration (FDA). The treatment guideline jointly issued by the VA and the Department of Defense (DoD) include two additional antidepressant medications, fluoxetine and venlafaxine, that have similarly limited effectiveness (Bernardy N C, et al. VA/DOD Clinical Practice Guideline for the Management of Posttraumatic Stress Disorder and Acute Stress Disorder. 2017, Washington, DC: United States Departments of Veterans Affairs and Defense). Second, there have been no new drugs approved by the FDA for the treatment of PTSD in almost two decades. The research and development of new drugs for PTSD has been limited, and current development may take many years to complete, resulting in a challenge to the field of how to proceed with identifying effective medications for PTSD treatment right now.

Leaders from the National Institute of Mental Health (NIMH) responded to the VA PTSD Psychopharmacology Work Group statement by noting that VA electronic health record (EHR) data provides “an unparalleled opportunity” to address the PTSD pharmacotherapy crisis (Gordon et al. (2017) Biol Psychiatry. 82:460-461). Their response highlighted the enormous potential of using large-scale EHR data to identify existing medications for other conditions that may also be effective medications for PTSD (Gordon et al. (2017) Biol Psychiatry. 82:460-461). There are several reasons why VA data is particularly well-suited to the discovery of novel pharmaceutical treatments for PTSD. First, PTSD is a common diagnosis among VA patients and, further, VA patients with PTSD have a high degree of medical comorbidity and receive a wide variety of medications to address these illnesses (Shiner et al. (2012) Mil Med. 177:814-822: Shiner et al. (2017) J Clin Psychiatry. 78: e545-e552). Second, the VA maintains national registry data sources capturing longitudinal patient care, including patient demographics, procedural and diagnostic codes, and pharmacy data. Notably, the VA's EHR repository contains data sources not typically available in large administrative datasets, including clinical note text and structured data from standardized mental health symptom assessments, allowing for the ability to identify the concurrent delivery of psychosocial treatments and examine changes in disorder severity over time.

### A. Methods and Materials

Study Sample and Data Sources. A nested case-control study was conducted within a previously established retrospective cohort that included all VA users with a clinical diagnosis of PTSD (International Classification of Diseases [ICD]-9:309.81, ICD-10: F43.1x) from Oct. 1, 1999 through Sep. 30, 2019 (Shiner et al. (2021) J Eval Clin Pract.). The data source contains information on services use, clinical diagnoses, filled prescriptions, and patient-reported outcome measures (PROMs) for PTSD using the PTSD Checklist (PCL) for these patients. This study was approved by the Veterans Institutional Review Board of Northern New England.

The goal of the current study was to evaluate any filled medications associated with PTSD symptom improvement, and as such, the analytic sample for this study included only patients with PROMs that were at least 30 days apart. 30 days was selected to allow for medications to be taken for at least one full course (typically 28 days). Patients with PROMs more than 365 days apart were excluded as these are unlikely to provide an accurate assessment of PTSD symptom change between the two time points. For patients who had two overlapping PROM intervals, those closest to 84 days in length were chosen to emulate typical PTSD medication efficacy trials (Watts et al. (2013) J Clin Psychiatry. 74: e541-550). When a patient had two or more overlapping intervals at exactly 84 days, the historically earliest interval was chosen. If patients had multiple non-overlapping intervals, all were included. Patients who met the selection criteria for PTSD in the original cohort and had at least one PROM pair where they started a new medication (defined below) constituted the overall analytic sample for the current study.

PTSD Improvement Cases and No Improvement Controls. In order to maximize the number of patients with available symptomatic measurement, two different versions of a PROM for PTSD were integrated into a single dataset (Shiner et al. (2021) J Eval Clin Pract.). The two PROMs were the PCL versions aligned to the Diagnostic and Statistical Manual of Mental Disorders (DSM), version IV and Version 5 (APA (2000): Diagnostic and Statistical Manual of Mental Disorders. 4th Edition. Text Revision: DSM-IV-TR. Arlington, VA: American Psychiatric Association: APA (2013): Diagnostic and Statistical Manual of Mental Disorders: Fifth Edition (DSM-5). Washington, D.C.: American Psychiatric Association), which will heretofore be called the PCL-IV and the PCL-5 (Blevins et al. (2015) J Trauma Stress. 28:489-498: Weathers et al. (1993) Trauma. Coping. and Adaptation. San Antonio, Texas: International Society for Traumatic Stress Studies, 9th Annual Meeting). Validation work shows a correlation of 0.87 between PCL versions in a large sample of Veterans (Bovin et al. (2016) Psychol Assess. 28:1379-1391). A validated crosswalk (ICC=0.96) was used to convert all values to PCL-5 scoring (Moshier et al. (Under review): An empirical crosswalk for the PTSD Checklist: Translating DSM-IV to DSM-5). PTSD improvement cases were defined by a clinically meaningful improvement, which was a decrease of 15 points or more between assessment points (Lee et al. (2019) Reliable Change Index and Clinically Significant Margins for the CAPS-5 and PCL-5 among Veterans. Boston, MA: International Society for Traumatic Stress Studies, 35th Annual Meeting). Because this improvement criterion was calculated using the Jacobson and Truax Reliable Change Index (1.96 times the standard error of the difference in change, which is the “distribution of change scores that would be expected if no actual change had occurred”: Jacobson and Truax (1991) J Consult Clin Psychol. 59:12-19), a corollary that changes of 7 points or less could be due to measurement error was used. Thus, the no PTSD improvement control group was defined as worsening of symptoms or reductions of up to 7 points on the PCL. Improvements between 8 and 14 points were of uncertain clinical significance and were therefore excluded.

Identification and Classification of New Medications. The predictors of interest were initiation of medications for any indication. Candidate agents included those that were started in the interval between PCL pairs and continued for at least a 28-day supply. When patients started multiple medications between a given PCL pair, they contributed information for each of the medications in the tree. To ensure that new medications were being examined, any agent prescribed in the six months prior to the first PCL score was excluded. All medications were classified by mechanism of action to create a phylogenic tree using ChEMBL, the European Bioinformatics Institute's open-source repository of bioactivity data (Bento et al. (2014) Nucleic Acids Res. 42: D1083-1090). Using the ChEMBL database, medications were grouped by mechanistic class, and these classes were used to create hierarchical tree structures for use. Individual medication trees connected at higher level branch points, thus creating one overarching phylogeny of all FDA-approved medications grouped according to mechanism. The resulting “Pharm Tree” was then converted into a log file for input into data mining software called TreeScan (described below).

Analytic Method: TreeScan. As this study was exploratory in nature, an innovative method was used to identify medications associated with cases of PTSD symptom improvement. The tree-based scan statistic (TreeScan) is data mining software that scans variables that exist in a natural hierarchy for associations with a dichotomous outcome, while adjusting for the multiple testing inherent in the large number of evaluated potential associations (Kulldorff et al. (2003) Biometrics. 59:323-331: Kulldorff et al. (2013) Pharmacoepidemiol Drug Saf. 22:517-523). In TreeScan, “leaves” represent the finest granular level of data and, in this application, corresponded to individual medications (e.g., clonidine). Groups of closely related “leaves” called “branches” represented higher-level drug mechanisms (e.g., alpha-2 adrenergic receptor agonists). TreeScan works by calculating a log likelihood ratio based on the number of observed and expected number of events for each leaf of the tree, as well as for each branch consisting of multiple, related leaves (Kulldorff et al. (2013) Pharmacoepidemiol Drug Saf. 22:517-523). The cuts that maximize the log likelihood ratio are identified as potential associations. Expected cases were calculated based on the overall distribution of cases in the entire tree. To obtain the number of expected cases for each medication, the proportion of patients who had improved PTSD symptoms among all patients in the study were multiplied by the number of patients who received each medication (population of the node). The ratio of observed to expected cases was calculated as a relative estimate of effect.

Medications with multiple mechanisms were grouped according to each individual mechanism, therefore one drug could appear in multiple trees. For example, trazodone (the medication most often prescribed to VA patients with PTSD: 6), is associated with three mechanisms in ChEMBL (i.e., serotonin 2a receptor antagonism, serotonin 2c receptor antagonism, serotonin transporter inhibition) and appears in three different branches with other medications that share the same mechanisms. It is possible that an association with improvement in PTSD symptoms could be observed at the level of the leaf representing an individual medication or at a higher-level branch representing a mechanism. The alerting threshold for TreeScan is based on a multiplicity-adjusted P value (Huybrechts et al. (2021) Am J Epidemiol. 190:1159-1168), which was set at 10%. Therefore, only outcome nodes with P<0.1 were considered statistical alerts.

Sensitivity analyses. To assess the possibility that changes in PTSD symptoms could be attributed to established interventions, the subset of patients in the analytic cohort who did not receive concurrent evidence-based treatment (EBT) for PTSD was identified to determine if the patterns of observed results in the overall analytic sample were consistent within this group. EBT was defined as evidence-based psychotherapies (EBPs) and evidence-based antidepressants (EBAs) as recommended by the VA-DoD clinical practice guideline (CPG) and commonly used in routine practice (Shiner et al. (2021) Adm Policy Ment Health. 48:70-87; Shiner et al. (2020) Adm Policy Ment Health. 47:451-467). While the 2017 VA-DoD CPG endorses many EBP protocols (he Management of Posttraumatic Stress Disorder Work Group (2017): VA/DoD Clinical Practice Guideline for the Management of Posttraumatic Stress Disorder and Acute Stress Disorder. Washington, DC: US Departments of Veterans Affairs and Defense), VA implementation efforts over the last 20 years have focused on two protocols including prolonged exposure (PE) and cognitive processing therapy (CPT: Rosen et al. (2016): Adm Policy Ment Health. 43:957-977). The provision of PE and CPT in the parent cohort was previously identified (Shiner et al. (2021) Improvements to PTSD quality metrics with natural language processing. J Eval Clin Pract). To identify provision of EBAs, filled prescriptions of fluoxetine, sertraline, paroxetine, or venlafaxine were identified. To ensure patients had current PTSD, all analyses were repeated within the subset of patients who had a baseline PCL of 38 or higher, as well as a clinical PTSD diagnosis within 30 days of the baseline PCL score. A score of 50 or higher on the PCL-IV has been frequently used as a case definition for military-related PTSD (Shiner et al. (2012) Mil Med. 177:814-8229: Terhakopian et al. (2008) J Trauma Stress. 21:290-300), and this is equivalent to a PCL-5 score of 36-38 (Moshier et al. (Under review): An empirical crosswalk for the PTSD Checklist: Translating DSM-IV to DSM-5: Hoge et al. (2014) Lancet Psychiatry. 1:269-277).

b. Results

There were 192,912 PCL pairs that met the inclusion criteria, representing 168,941 patients (Table 1). There was a median of 89 days between the baseline and follow-up PCL, with 25% of intervals spanning less than 79 days and 25% of intervals spanning more than 141 days. Cases and controls were similar in terms of demographic and diagnostic characteristics with standardized mean differences of well below 0.1 (Table 2). Cases were more likely to have received EBT and had higher baseline PCL scores. When the cohort was restricted to those with no EBT receipt, there were 75,266 PCL pairs representing 69,453 patients. When the cohort was restricted to those with a baseline PCL of 38 or higher as well as a PTSD diagnosis within 30 days, there were 134,289 PCL pairs representing 119,372 patients overall and 47,778 PCL pairs representing 44,400 patients in the subgroup with no EBT receipt.

### Table 1.

TABLE 1
Cohort Selection

| | |
|---|---|
| | PCL |
| Patients | Pairs |

Overall Cohort

| | | | |
|---|---|---|---|
| A | How many patients had any diagnosis of PTSD between | 2,098,389 | N/A |
| | Oct. 1, 1999 and Sep. 30, 2019? | | |
| B | How many of (A) had at least 1 PCL Score? | 852,438 | N/A |
| C | How many of (B) had at least 1 additional PCL score within | 353,847 | 6,158,258 |
| | 30 to 365 days of the first? | | |
| D | How many of (C) do not include overlapping PCL pairs | 353,847 | 480,372 |
| | within the same patient? | | |
| E | How many of (D) had at least one concurrent drug trial | 192,006 | 223,048 |
| | lasting at least 28 days? | | |
| F | How many of (E) met the criteria to be considered a case* or | 168,941 | 192,912 |
| | control**? | | |

Sensitivity Analysis Cohorts

| | | | |
|---|---|---|---|
| G | How many of (F) did not coincide with any instance of | 69,453 | 75,266 |
| | EBT? | | |
| H | How many of (F) had a baseline PCL score of 38 or higher | 119,372 | 134,289 |
| | and a PTSD diagnosis within 30 days of baseline? | | |
| I | How many of (H) did not coincide with any instance of | 44,400 | 47,778 |
| | EBT? | | |

PTSD = Posttraumatic Stress Disorder,
PCL = PTSD Checklist,
EBT = Evidence-Based Treatment for PTSD
*Case: PCL improvement of 15 points or more
**Control: PCL improvement of 7 points or less or PCL worsening

### Table 2.

TABLE 2
Characteristics of the Sample

| | Overall | | Cases* | | Controls** | | |
|---|---|---|---|---|---|---|---|
| | N = 192,912 | | n = 43,402 | | n = 149,510 | | |
| Variable | % | n | % | n | % | n | SMD |

Demographic Characteristics at Baseline

| | | | | | | | |
|---|---|---|---|---|---|---|---|
| Age (M ± SD) | 44.24 | 14.01 | 44.13 | 14.00 | 44.27 | 14.01 | 0.010 |
| Female | 14.33 | 27,641 | 15.88 | 6,892 | 13.88 | 20,749 | 0.056 |
| White Non-Hispanic | 60.01 | 115,757 | 61.00 | 26,477 | 59.72 | 89,280 | 0.026 |
| Black Non-Hispanic | 20.99 | 40,501 | 20.94 | 9,087 | 21.01 | 31,414 | 0.002 |
| Hispanic | 11.36 | 21,924 | 10.74 | 4,663 | 11.55 | 17,261 | 0.025 |
| Post-9/11 Era Veteran | 58.72 | 113,276 | 56.37 | 24,465 | 59.40 | 88,811 | 0.061 |
| Vietnam Era Veteran | 18.36 | 35,412 | 18.11 | 7,861 | 18.43 | 27,551 | 0.008 |
| Combat Exposure | 63.50 | 122,508 | 60.87 | 26,419 | 64.27 | 96,089 | 0.070 |
| Military Sexual Trauma Exposure | 16.26 | 31,364 | 17.79 | 7,722 | 15.81 | 23,642 | 0.053 |

Comorbidities in the Two Years Prior to Baseline

| | | | | | | | |
|---|---|---|---|---|---|---|---|
| Depressive Disorders | 66.75 | 128,763 | 67.80 | 29,427 | 66.44 | 99,336 | 0.029 |
| Anxiety Disorders | 39.62 | 76,425 | 40.87 | 17,737 | 39.25 | 58,688 | 0.033 |
| Bipolar Disorders | 8.05 | 15,528 | 9.12 | 3,960 | 7.74 | 11,568 | 0.050 |
| Psychotic Disorders | 4.31 | 8,315 | 4.73 | 2,054 | 4.19 | 6,261 | 0.026 |
| Personality Disorders | 6.77 | 13,058 | 7.29 | 3,164 | 6.62 | 9,894 | 0.026 |
| Substance Use Disorders | 33.99 | 65,573 | 36.89 | 16,010 | 33.15 | 49,563 | 0.078 |

Concurrent Evidence Based PTSD Treatment

| | | | | | | | |
|---|---|---|---|---|---|---|---|
| Any Sessions of EBP for PTSD | 26.48 | 51,092 | 38.57 | 16,740 | 22.98 | 34,352 | 0.343 |
| Any Prescriptions for EBA for PTSD | 46.12 | 88,974 | 47.06 | 20,423 | 45.85 | 68,551 | 0.024 |
| Any EBA or EBP for PTSD | 60.98 | 117,646 | 68.62 | 29,784 | 58.77 | 87,862 | 0.206 |

PTSD Checklist (PCL) Information

| | | | | | | | |
|---|---|---|---|---|---|---|---|
| Baseline Score (M ± SD) | 49.02 | 16.40 | 56.05 | 12.70 | 46.98 | 16.78 | 0.610 |
| Baseline Score 38+ | 76.10 | 146,812 | 91.21 | 39,589 | 71.72 | 107,223 | 0.518 |
| PTSD Diagnosis within | 89.48 | 172,619 | 91.85 | 39,865 | 88.79 | 132,754 | 0.104 |
| 30 Days of Baseline PCL | | | | | | | |
| PTSD Diagnosis within | 69.61 | 134,289 | 84.20 | 36,544 | 65.38 | 97,745 | 0.444 |
| 30 Days of Baseline PCL | | | | | | | |
| and Baseline Score of 38+ | | | | | | | |

SMD = Standardized Mean Difference, PTSD = Posttraumatic Stress Disorder, EBP = Evidence-Based Psychotherapy, EBA = Evidence-Based Antidepressants
*Case: PCL improvement of 15 points or more
**Control: PCL improvement of 7 points or less or PCL worsening

Medications Associated with Improvement in PTSD Symptoms. Among all PCL pairs, TreeScan identified 25 unique associations between medications and improvements in PTSD symptoms. Although medications were grouped according to mechanism of action in the TreeScan hierarchy, the resulting medication signals were organized according to clinical indication for ease of interpretation (Table 3). Sertraline, which is FDA-approved for the treatment PTSD, was prescribed widely but had a weak overall association with symptom improvement (ratio of observed to expected [O:E]=1.16; p=0.001). Other medications commonly prescribed for PTSD and related mood disorders like citalopram, fluoxetine, trazodone, and lithium had similarly small associations with PTSD symptom improvement. Prazosin, which is commonly prescribed for PTSD-related nightmares also had a similar effect (O:E=1.10; P=0.001). Atypical antipsychotics have fallen in and out of favor as treatments for PTSD (Huang et al. (2020) Front Pharmacol. 11:559), and several small associations were observed at the branch level for these medications (e.g., the serotonin 2C receptor antagonist branch; O:E=1.04; P=0.01).

### Table 3.

TABLE 3
Medications Associated with Improvement in PTSD,*
Among All Patients in the Overall Sample

| TreeScan Node | OBS | EXP | O:E | P |
|---|---|---|---|---|
| Antivirals | | | | |
| Hepatitis C Virus | 43 | 24 | 1.77 | 0.02 |
| NS3/4A Protease Inhibitor | | | | |
| ¹Glecaprevir | 33 | 16 | 2.09 | 0.005 |
| Hepatitis C Virus | 100 | 64 | 1.57 | 0.001 |
| NS5A Protein Inhibitor | | | | |
| ¹Pibrentasvir | 33 | 16 | 2.09 | 0.005 |
| Velpatasvir | 24 | 11 | 2.10 | 0.04 |
| Antipsychotics | | | | |
| Serotonin 2A (5-HT2A) | 10511 | 10102 | 1.04 | 0.001 |
| Receptor Antagonist | | | | |
| Serotonin 2C (5-HT2C) | 9630 | 9281 | 1.04 | 0.01 |
| Receptor Antagonist | | | | |
| Addiction Treatments | | | | |
| Opioid (Mu/Kappa/Delta) | 1031 | 815 | 1.27 | 0.001 |
| Receptor Antagonist | | | | |
| Naloxone | 426 | 312 | 1.37 | 0.001 |
| Naltrexone | 605 | 504 | 1.20 | 0.001 |
| Supplements | | | | |
| Folic Acid | 567 | 445 | 1.27 | 0.001 |
| Thiamine | 444 | 351 | 1.27 | 0.001 |
| Cholecalciferol | 2647 | 2470 | 1.07 | 0.02 |
| Mood Disorder Treatments | | | | |
| Serotonin Transporter Inhibitor | 17417 | 16196 | 1.09 | 0.001 |
| Sertraline | 4659 | 4025 | 1.16 | 0.001 |
| Citalopram | 1653 | 1508 | 1.10 | 0.01 |
| Fluoxetine | 1716 | 1571 | 1.09 | 0.02 |
| Trazodone | 4308 | 4061 | 1.06 | 0.005 |
| Lithium | 304 | 246 | 1.24 | 0.02 |
| Sleep Disorder Treatments | | | | |
| Adrenergic Alpha1 Receptor Antagonist | 5279 | 4960 | 1.07 | 0.001 |
| Prazosin | 4659 | 4259 | 1.10 | 0.001 |
| Melatonin Receptor Agonist | 1326 | 1190 | 1.12 | 0.005 |
| Melatonin | 1295 | 1161 | 1.12 | 0.006 |
| Cognitive Stimulants | | | | |
| Nicotine | 2371 | 2109 | 1.13 | 0.001 |
| Atomoxetine | 104 | 74 | 1.40 | 0.08 |

PTSD = Posttraumatic Stress Disorder;
OBS = Observed Cases;
EXP = Expected Cases;
O:E = Observed Cases over Expected Cases
*Improved by 15 or more points (compared to 7 points or less or PCL worsening) on the PTSD Checklist.
¹Glecaprevir/Pibrentasvir = Mavyret (fixed-dose tablet of Glecaprevir 100 mg and Pibrentasvir 40 mg)

Several drugs commonly used in detoxification and addiction treatment demonstrated associations. Two opioid antagonists, naloxone (O:E=1.37: P=0.001) and naltrexone (O:E=1.20; P=0.001), were associated with improvements in PTSD symptoms. Similarly, folic acid (O:E=1.27; P=0.001) and thiamine (O:E=1.27: P=0.001) were associated with improvements in PTSD symptoms. The drugs with the strongest associations with PTSD symptom improvement were Hepatitis C Virus (HCV) Direct Acting Antivirals (DAA) including glecaprevir (O:E=2.09; P=0.005), pibrentasvir (O:E=2.09; P=0.005), and velpatasvir (O:E=2.10, P=0.04). Of note, glecaprevir and pibrentasvir demonstrated associations that were identical in magnitude, which is explained by the fact that they are co-prescribed under the brand name Mavyret.

Sensitivity Analyses. Among the subset of PCL pairs that did not include EBT (no sessions of EBP, no prescriptions for EBA), TreeScan identified 19 unique associations representing cuts at the leaf or branch level (Table 4). Again, the strongest associations were HCV DAAs including Mavyret (O:E=3.09; P=0.01), indicating that the associations detected using TreeScan in the overall sample were not due to the effect of established PTSD interventions. Results were similar when the cohort was restricted to those with a baseline PCL of 38 or higher as well as a PTSD diagnosis of 30 days, with O:E ratios for Mavyret of 2.21 (P=0.001) in the overall group and 3.52 (P=0.001) in the subgroup with no EBT.

### Table 4.

TABLE 4
Medications Associated with Improvement in PTSD,*
Among Patients in the Overall Sample without
Any Concurrent Evidence-Based Treatment

| TreeScan Node | OBS | EXP | O:E | P |
|---|---|---|---|---|
| Antivirals | | | | |
| Hepatitis C Virus NS3/4A Protease Inhibitor | 19 | 7 | 2.65 | 0.01 |
| ¹Glecaprevir | 14 | 5 | 3.09 | 0.01 |
| Hepatitis C Virus NS5A Protein Inhibitor | 41 | 21 | 1.99 | 0.003 |
| ¹Pibrentasvir | 14 | 5 | 3.09 | 0.01 |
| Antipsychotics | | | | |
| Serotonin 2A (5-HT2A) Receptor Antagonist | 3616 | 3225 | 1.14 | 0.001 |
| Serotonin 2C (5-HT2C) Receptor Antagonist | 3285 | 2953 | 1.12 | 0.001 |
| Dopamine D2 Receptor Antagonist | 819 | 690 | 1.19 | 0.001 |
| Quetiapine | 460 | 371 | 1.24 | 0.001 |
| Addiction Treatments | | | | |
| Opioid (Mu/Kappa/Delta) | 306 | 242 | 1.27 | 0.003 |
| Receptor Antagonist | | | | |
| Supplements | | | | |
| Folic Acid | 185 | 134 | 1.38 | 0.001 |
| Thiamine | 146 | 106 | 1.38 | 0.01 |
| Mood Disorder Treatments | | | | |
| Serotonin Transporter Inhibitor | 3631 | 3227 | 1.14 | 0.001 |
| Citalopram | 1039 | 814 | 1.28 | 0.001 |
| Escitalopram | 558 | 474 | 1.18 | 0.01 |
| Trazodone | 1272 | 1157 | 1.1 | 0.05 |
| Lithium | 127 | 92 | 1.38 | 0.03 |
| Mirtazapine | 849 | 755 | 1.13 | 0.05 |
| Sleep Disorder Treatments | | | | |
| Adrenergic Alpha1 Receptor Antagonist | 1593 | 1418 | 1.13 | 0.001 |
| Prazosin | 1375 | 1170 | 1.18 | 0.001 |

OBS = Observed Cases;
EXP = Expected Cases;
O:E = Observed Cases over Expected Cases
Evidence-Based Treatment for PTSD included prolonged exposure, cognitive processing therapy, fluoxetine, sertraline, paroxetine, and venlafaxine.
*Improved by 15 or more points (compared to 7 points or less or PCL worsening) on the PTSD Checklist.
¹Glecaprevir/Pibrentasvir = Mavyret (fixed-dose tablet of Glecaprevir 100 mg and Pibrentasvir 40 mg)

See also Tables 5 and 6 below.

### Table 5.

TABLE 5
Standardized Morbidity Ratio for Medications Associated
with Improvement in PTSD,* among all Patients
with a Baseline PCL Score of 38 or Higher as
well as a PTSD Diagnosis within 30 Days

| TreeScan Node | OBS | EXP | O:E | P |
|---|---|---|---|---|
| Antivirals | | | | |
| Hepatitis C Virus | 36 | 20 | 1.78 | 0.03 |
| NS3/4A Protease Inhibitor | | | | |
| ¹Glecaprevir | 30 | 14 | 2.21 | 0.001 |
| Hepatitis C Virus | 84 | 51 | 1.65 | 0.001 |
| NS5A Protein Inhibitor | | | | |
| ¹Pibrentasvir | 30 | 14 | 2.21 | 0.001 |
| Velpatasvir | 21 | 10 | 2.17 | 0.02 |
| Addiction Treatments | | | | |
| Opioid (Mu/Kappa/Delta) | 912 | 723 | 1.26 | 0.001 |
| Receptor Antagonist | | | | |
| Naloxone | 376 | 279 | 1.35 | 0.001 |
| Naltrexone | 536 | 444 | 1.21 | 0.001 |
| Supplements | | | | |
| Folic Acid | 469 | 391 | 1.20 | 0.003 |
| Thiamine | 377 | 304 | 1.24 | 0.001 |
| Cholecalciferol | 2238 | 2070 | 1.08 | 0.008 |
| Mood Disorder Treatments | | | | |
| Serotonin Transporter Inhibitor | 14605 | 13719 | 1.08 | 0.001 |
| Citalopram | 1315 | 1202 | 1.10 | 0.04 |
| Sertraline | 3909 | 3434 | 1.14 | 0.001 |
| Trazodone | 3610 | 3396 | 1.07 | 0.008 |
| Sleep Disorder Treatments | | | | |
| Melatonin Receptor Agonist | 1158 | 1058 | 1.10 | 0.09 |
| Melatonin | 1131 | 1033 | 1.10 | 0.09 |
| Cognitive Stimulants | | | | |
| Nicotine | 2007 | 1736 | 1.16 | 0.001 |
| Atomoxetine | 92 | 65 | 1.41 | 0.05 |

PTSD = Posttraumatic Stress Disorder;
OBS = Observed Cases;
EXP = Expected Cases;
O:E = Observed Cases over Expected Cases
*Improved by 15 or more points (compared to 7 points or less or PCL worsening) on the PTSD Checklist.
¹Glecaprevir/Pibrentasvir = Mavyret (fixed-dose tablet of Glecaprevir 100 mg and Pibrentasvir 40 mg)

### Table 6.

TABLE 6
Standardized Mortality Ratio for Medications Associated with
Improvement in PTSD,* among Patients with a Baseline PCL
Score of 38 or Higher as well as a PTSD Diagnosis within
30 Days and without Any Concurrent Evidence-Based Treatment

| TreeScan Node | OBS | EXP | O:E | P |
|---|---|---|---|---|
| Antivirals | | | | |
| Hepatitis C Virus NS3/4A Protease Inhibitor | 16 | 6 | 2.85 | 0.004 |
| ¹Glecaprevir | 14 | 4 | 3.52 | 0.001 |
| Hepatitis C Virus NS5A Protein Inhibitor | 32 | 15 | 2.21 | 0.001 |
| ¹Pibrentasvir | 14 | 4 | 3.52 | 0.001 |
| Antipsychotics | | | | |
| Serotonin 2A (5-HT2A) Receptor Antagonist | 2903 | 2656 | 1.10 | 0.001 |
| Serotonin 2C (5-HT2C) Receptor Antagonist | 2634 | 2417 | 1.10 | 0.001 |
| Addiction Treatments | | | | |
| Opioid (Mu/Kappa/Delta) | 256 | 205 | 1.25 | 0.02 |
| Receptor Antagonist | | | | |
| Mood Disorder Treatments | | | | |
| Serotonin Transporter Inhibitor | 2841 | 2574 | 1.12 | 0.001 |
| Citalopram | 785 | 632 | 1.25 | 0.001 |
| Trazodone | 992 | 893 | 1.12 | 0.03 |
| Cognitive Stimulants | | | | |
| Nicotine | 610 | 522 | 1.17 | 0.005 |

OBS = Observed Cases;
EXP = Expected Cases;
O:E = Observed Cases over Expected Cases
Evidence-Based Treatment for PTSD included prolonged exposure, cognitive processing therapy, fluoxetine, sertraline, paroxetine, and venlafaxine.
*Improved by 15 or more points (compared to 7 points or less or PCL worsening) on the PTSD Checklist.
¹Glecaprevir/Pibrentasvir = Mavyret (fixed-dose tablet of Glecaprevir 100 mg and Pibrentasvir 40 mg)

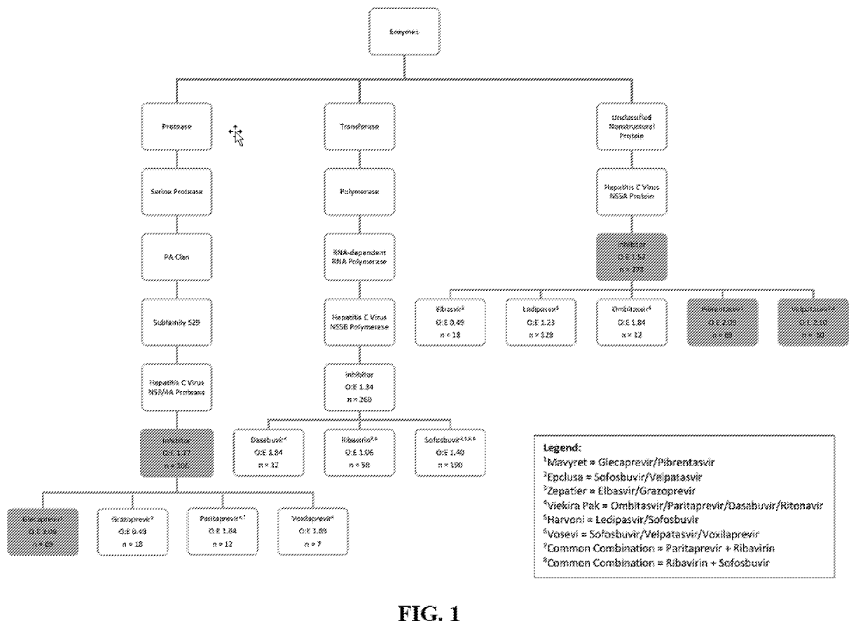

Illustrating Agents with Associations and Related Agents. To illustrate the mechanistic hierarchy of TreeScan and aid in conceptualization of these results, a simplified phylogenic tree containing the branches (mechanistic classes) and leaves (individual drugs) for which improvement in PTSD symptoms was identified in the overall results was recreated. The tree is not drawn to scale or all inclusive: it only represents a small cross-section of medications within a particular mechanistic class that demonstrated associations in the overall TreeScan models. The phylogenic tree highlights HCV DAAs (FIG. 1) with three main branches (mechanisms): HCV nonstructural protein 3/4A (NS3/4A) protease inhibitors, nonstructural protein 5B (NS5B) RNA-dependent RNA polymerase inhibitors, and nonstructural protein 5A (NS5A) inhibitors. Cuts (branches and leaves) identified by TreeScan in the overall analysis are highlighted. The figure legend outlines common combinations of DAAs listed in this tree.

Similar to glecaprevir and pibrentasvir (Mavyret), grazoprevir and elbasvir are listed in separate branches but demonstrate effects that are identical in magnitude because they are prescribed together as Zepatier. However, sofosbuvir can be combined with four different DAAs listed in this tree, explaining the relatively large number of observations for this leaf. Sofosbuvir was most commonly prescribed with ledipasvir as Harvoni, which did not generate a statistical alert (O:E=1.23, P>0.1). Among the three mechanistic branches, the NS3/4A protease inhibitors (O:E=1.77, P=0.02) demonstrated the largest effect size followed by the NS5A protein inhibitors (O:E=1.57, P=0.001). Within the NS5A inhibitor branch, both pibrentasvir and velpatasvir had strong associations with improvement in PTSD symptoms. Velpatasvir is prescribed in combinations including Epclusa (velpatasvir and sofosbuvir) and Vosevi (velpatasvir, sofosbuvir, and voxilaprevir), and there were no associations for other medications prescribed along with velpatasvir. This introduces the possibility that the NS5A inhibitor branch drives the DAA finding and the signal for the NS3/4A protease inhibitor branch is incidental, as glecaprevir is always prescribed with the NS5A inhibitor pibrentasvir.

c. Discussion

This study is the first large-scale exploration of existing medications that may have potential efficacy for the treatment of PTSD using a population-based sample. There has been limited advancement in the pharmacologic treatment options for PTSD over the last two decades, and the findings from this study are an initial step towards future critical innovation in this area. To address this issue, a novel exploratory method called TreeScan was used. Although the application of any novel exploratory method can have limitations, several features of these results indicate that TreeScan worked as intended. As expected, it was found that the serotonin transport inhibitors commonly used and recommended for the treatment of PTSD improved PTSD symptoms in this study. Improvements associated with medications known to enhance the effects of serotonin transport inhibitors, such as lithium (Stamm T J et al. (2008) Psychiatr Genet. 18:92-97), were also observed. However, consistent with the existing PTSD literature and clinical experience, the observed effects were small. Similarly, although prazosin is often prescribed to treat nightmares associated with PTSD, the effect on overall PTSD symptoms was limited. Previous research has identified a strong association between PTSD and substance use disorders (SUD), necessitating the development of treatments that address both conditions (Sofuoglu et al. (2014) Addictive Behaviors. 39:428-433). Not surprisingly, medications closely associated with alcohol detoxification, like folic acid and thiamine, and addiction treatments, like naloxone and naltrexone, were associated with small improvements in PTSD symptoms. This is especially clear in the case of naloxone, which is prescribed as an emergency kit and assigned as a 30-day supply in the ordering system, but only taken in the case of an overdose. It is possible that possessing this potentially life-saving tool results in a substantial decrease in anxiety. Further, supplements like folic acid and thiamine are commonly initiated as part of initial detoxification management and continued orally as part of aftercare. Therefore, it is likely that more general effects of patients initiating substance abuse treatment are being observed, rather than a direct effect of opioid antagonism.

Consistent with the goals of this study, a previously unknown association was found between use of several non-psychotropic medications and improvement in PTSD symptoms. The sensitivity analyses indicate that these improvements are not due to concurrent receipt of established PTSD treatments. The strongest associations came from the DAAs. These agents have revolutionized the care of HCV: in contemporary practice they provide a cure for HCV infections in most cases (Backus et al. (2017) Antivir Ther. 22:481-493: Ioannou et al. (2016) Gastroenterology. 151:457-471 e455). These DAAs are most commonly prescribed as combinations to avoid antiviral resistance (Zając et al. (2019) European Journal of Medicinal Chemistry. 165:225-249). There is growing evidence that HCV has direct effects on the brain, leading to neuropsychiatric symptoms (Iriana et al. (2017) Clin Liver Dis. 21:535-542). As opposed to interferon-containing HCV treatment regimens that were known to cause or worsen depression (Machado et al. (2017) J Affect Disord. 209:235-245: Udina et al. (2012) J Clin Psychiatry. 73:1128-1138), available studies suggest that DAA treatment without interferon does not cause or worsen depression and may lead to improvements in depression and related mental health symptoms (Gallach et al. (2018) C. PLOS One. 13: e0208112: Hahn et al. (2018) Eur J Clin Invest. 48: e13024; Kesen et al. (2019) Turk J Gastroenterol. 30:801-806; Pericot-Valverde et al. (2020) Open Forum Infect Dis. 7: ofaa380). Prior studies have not evaluated the effect of DAA treatment on PTSD symptoms or compared the effects of various DAA combinations on mental health symptoms. If HCV is contributing to PTSD symptoms, some DAAs may be more effective than others because they are better at clearing HCV virus in the brain. However, the most prescribed combinations in this analysis, Harvoni and Mavyret, are both highly effective for HCV, but appear to have drastically different effects on PTSD symptoms. This finding introduces the possibility that several DAAs may improve PTSD symptoms through a mechanism unrelated to clearance of HCV.

Although the mechanism of action of individual DAAs at the hepatocyte has recently been explored (Zając et al. (2019) European Journal of Medicinal Chemistry. 165:225-249), relatively little is known about their off-target effects. However, there are several mechanisms that could explain behavioral effects, including PTSD symptom improvement for these medications. First, pibrentasvir falls into a class of drugs called phenylpiperidines (Wishart et al. (2018) Nucleic Acids Res. 46: D1074-D1082), which includes synthetic opioids (Elbaridi et al. (2017) Pain Physician. 20: Se23-se31). Disruptions in the endogenous opioid system has been postulated in the pathophysiology of PTSD (van der Kolk B A (1994) Harv Rev Psychiatry. 1:253-265), and there are high rates of opioid use disorders and pain disorders in the population studied (Shiner et al. (2017) J Dual Diagn. 13:201-212). Second, pibrentasvir inhibits NS5A, a non-structural HCV protein that binds to and inactivates protein kinase R (PKR: He et al. (2001) J Virol. 75:5090-5098). PKR has a central role in cellular response to stress. Thus, pibrentasvir inhibition of NS5 A or an unknown cellular homologue may result in modified PKR activity leading to alterations in gene expression profiles across diverse cell types. As PKR is activated by pro-inflammatory cytokines that disrupt the blood brain barrier, PKR has been linked with neuroinflammation and neurodegenerative disease, and inhibition of PKR may therefore exert a neuroprotective effect (Gal-Ben-Ari et al. (2019) Frontiers in Molecular Neuroscience. 11). PKR inactivation has been associated with enhanced memory and learning in animal models (Zhu et al. (2011) Cell. 147:1384-1396). As PTSD symptom improvement has been conceptualized as new learning, these medications may promote symptom improvement through this enhanced learning (Fonzo et al. (2021) Biol Psychiatry. 89:857-867). However, the differential pattern of PTSD response among NS5A inhibitors suggests that other unknown factors such as blood brain barrier permeability are also relevant. Finally, it remains within the realm of possibility that some form of viral illness is involved in at least some cases of PTSD and DAAs may have differential effects on that as-yet-unidentified pathogen.

In conclusion, this exploratory approach both demonstrated findings consistent with what is known about pharmacotherapy for PTSD and uncovered a novel medication that may improve PTSD symptoms. Those novel medications demonstrated larger association with PTSD improvement than standard PTSD treatments typically used in clinical practice. Because these novel medications do not have known effects on typical monoamines associated with PTSD treatments (i.e., serotonin and norepinephrine), this association would indicate a gap either in the understanding of the psychotropic effects of some DAAs or in the pathophysiology of PTSD.

### 2. Comparative Effectiveness of Direct-Acting Antivirals for Posttraumatic Stress Disorder

As detailed above, an exploratory study using United States (US) Department of Veterans Affairs (VA) medical records was conducted, and an unexpected association between receipt of several direct acting antiviral (DAA) medications used in the treatment of Hepatitis C Virus (HCV) and improvement in PTSD symptoms was uncovered. See also Shiner et al. (2021) “Mining Clinical Data for Novel PTSD Medications.” Biol Psychiatry. Three agents, including glecaprevir, pibrentasvir, and velpatasvir, were all associated with over double the expected number of patients experiencing a clinically meaningful improvement in PTSD symptoms (observed to expected ratio >2.0). As a point of comparison, sertraline, which is FDA-approved for PTSD, was associated with only a slightly higher than expected improvement (observed to expected ratio=1.2). DAA treatment is exceptionally effective for HCV, with the VA reporting cure rates of greater than 90% (Belperio et al. (2017) Ann Intern Med. 167 (7): 499-504). While it is possible that patients' PTSD symptoms improved when they were cured of HCV, a differential pattern of PTSD response was found among patients receiving DAA regimens that have similar efficacy for HCV. Glecaprevir and pibrentasvir (GLE/PIB) are always prescribed together under the brand name Mavyret. Velpatasvir is commonly prescribed in combination with sofosbuvir (SOF/VEL) under the brand name Epclusa. Sofosbuvir is also commonly prescribed with ledipasvir (LDV/SOF) under the brand name Harvoni. Neither sofosbuvir nor ledipasvir were associated with a significantly higher than expected improvement in PTSD symptoms. Though the effects of DAA combinations on the hepatocyte have been studied extensively (Zajac et al. (2019) Eur J Med Chem. 65:225-49), little is known about their psychotropic effects. Without wishing to be bound by theory, it was postulated that several potential mechanisms related to cellular signaling could be involved in ameliorating PTSD symptoms, and that differences between the agents could be explained by exogenous factors such as blood brain barrier (BBB) permeability (Shiner et al. (2021) “Mining Clinical Data for Novel PTSD Medications.” Biol Psychiatry). If this exploratory finding is confirmed in subsequent analyses using causal methods, the discovery that several DAAs have an off-target effect on PTSD symptoms could inform both the deployment of a new class of PTSD medications and an improved understanding of PTSD pathophysiology.

While the strongest evidence for the efficacy of DAAs in treating PTSD would be prospective RCTs in patients who have PTSD without HCV, additional analysis of existing medical records data can also provide useful information, in a resource and time efficient manner (Shiner and Watts (2020) Am J Epidemiol. 190 (7): 1220-1222). To that end, several VA registry-based observational studies were completed, which compare established medications for PTSD, providing a template to evaluate novel agents using real-world data (Shiner et al. (2020) J Clin Psychiatry 81 (6): Shiner et al. (2018) J Clin Psychiatry 79 (5): Shiner et al. (2021) J Clin Psychiatry 82 (6)). A similar registry-based study focusing on the effect of DAAs for PTSD could rule out some sources of bias, narrow the list of candidate agents, and provide a preliminary estimate of effectiveness. In addition to the possibility that improvements in PTSD symptoms could be related more directly to curing HCV, there are other sources of bias that could have impacted the exploratory study results (Shiner et al. (2021) “Mining Clinical Data for Novel PTSD Medications.” Biol Psychiatry). First, patients were non-randomly assigned to DAA treatment. For example, the DAA combinations that were FDA approved first (e.g., LDV/SOF in 2014) were given to many chronically ill patients (Belperio et al. (2017) Ann Intern Med. 167 (7): 499-504), while DAA combinations that were approved later (e.g., SOF/VEL in 2016 and GLE/PIB in 2017) may have been given more often to patients with new disease because chronically ill patients were already cured. This could lead to differences in age, gender, PTSD chronicity, and other factors between patients who receive each DAA combination. Second, it is possible patients received other treatments that affected their PTSD symptoms. Although the previous study included a sensitivity analysis that removed patients who received evidence-based PTSD treatments recommended by the VA including several trauma-focused psychotherapies and antidepressants (Shiner et al. (2021) “Mining Clinical Data for Novel PTSD Medications.” Biol Psychiatry), it remains possible that patients received additional treatments for co-occurring mental health disorders that led to improvement in PTSD symptoms.

As a next step in evaluating the effect of DAAs on PTSD symptoms, a VA registry-based study was performed, comparing the three most commonly prescribed DAA combinations (Shiner et al. (2021) “Mining Clinical Data for Novel PTSD Medications.” Bio/Psychiatry), including LDV/SOF, SOF/VEL, and GLE/PIB. To control for possible confounding, a weighting approach was used that accounted for patients' propensity to receive each DAA combination based on a set of known covariates related to hepatitis disease status, other medical and mental illness, and concurrent mental health treatments. Without wishing to be bound by theory, it was hypothesized that patients receiving SOF/VEL and GLE/PIB would have superior PTSD symptomatic outcomes compared to those receiving LDV/SOF.

a. Methods

Data Sources. The VA Corporate Data Warehouse (CDW) was used to identify all VA users with a clinical diagnosis of PTSD (309.81, F43.1x) from Oct. 1, 1999-9/30/2019. Information was obtained on services use, clinical diagnoses, prescription fills, laboratory tests, and patient-reported outcome measures (PROMs) from the CDW for these patients. This study was approved by the Veterans Institutional Review Board of Northern New England.

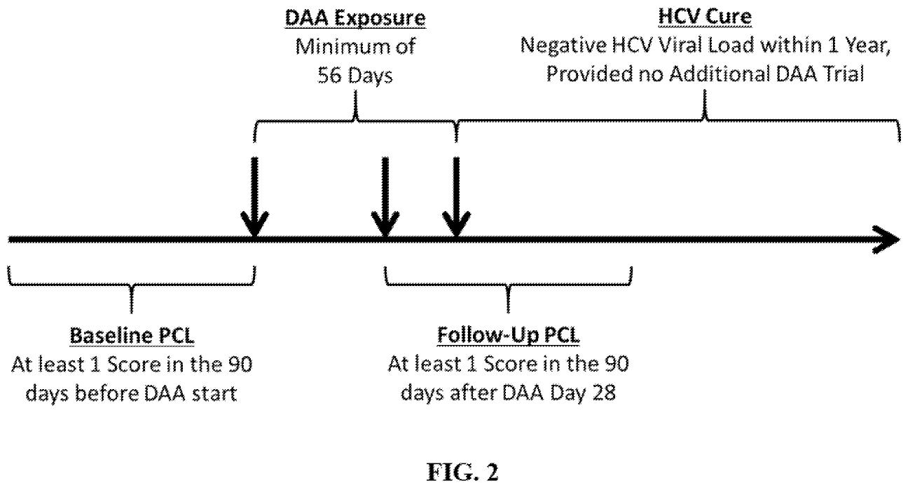

Direct Acting Antiviral Cohort Selection. Patients who completed a course of LDV/SOF, SOF/VEL, and GLE/PIB were identified (FIG. 2). At least 56 days of continuous treatment was required. The cohort was restricted to those who received baseline PTSD symptom measurement within 90 days prior to DAA initiation and follow-up PTSD symptom measurement after a minimum of 28 days of continuous DAA receipt. 28 days was chosen as it was the minimum exposure for a drug to be included in the exploratory study because prior information about the etiologically relevant outcome window was not available. Up to an additional 90 days was allowed to capture the follow-up PTSD symptom measure. Though not required for cohort inclusion, a sensitivity analysis was further conducted to examine effects among only patients who were cured of HCV to determine if HCV cure accounts for observed associations. Cure was defined as an undetectable HCV viral load up to a year after the completion of DAA therapy, provided there was no additional course of DAA.

PTSD Symptoms. To maximize sample size within the clinical subgroups, two different versions of a PROM were integrated for PTSD, captured from up to two data sources within the CDW, to obtain the baseline and follow-up symptom measurements. This included scores obtained from structured data produced by psychometric assessment software in the VA medical record and scores documented by clinicians in their treatment notes. A previously published natural language processing (NLP) algorithm was used, with 98% precision in identifying the correct score and version of the PCL to abstract scores from clinical notes (Holder et al. (2020) J Affect Disord. 273:425-33: Holder et al. (2020) Behav Res Ther. 135:103756). Scores abstracted from structured data and from NLP of clinical notes were integrated into a single dataset, which has been described in detail elsewhere (Shiner et al. (2021) “Improvements to PTSD quality metrics with natural language processing.” J Eval Clin Pract.).

Briefly, the two PROMs were the PTSD Checklist (PCL) versions aligned to the Diagnostic and Statistical Manual of Mental Disorders (DSM), version IV and Version 5 (American Psychiatric Association. Diagnostic and Statistical Manual of Mental Disorders: Fifth Edition (DSM-5). American Psychiatric Association: Washington, D.C.: 2013: American Psychiatric Association. Diagnostic and Statistical Manual of Mental Disorders, 4th Edition, Text Revision: DSM-IV-TR. American Psychiatric Association: Arlington, VA: 2000), which will heretofore be called the PCL-IV and the PCL-5 (Blevins et al. (2015) J Trauma Stress. 28 (6): 489-98: Weathers et al. in Trauma. Coping. and Adaptation (International Society for Traumatic Stress Studies, 9th Annual Meeting, San Antonio, Texas, 1993). Validation work shows a correlation of 0.87 between PCL versions in a large sample of Veterans (Bovin et al. (2016) Psychol Assess. 28 (11): 1379-91). A validated crosswalk (ICC=0.96) was used to convert all values to PCL-5 scoring (Moshier et al. (2019) J Trauma Stress. 32 (5): 799-805). To preserve sample size, a minimum severity score was not required. However, a covariate was created for baseline severity score of ≥31 out of 80, as scores of 31-33 are optimally efficient for diagnosing PTSD (Bovin et al. (2016) Psychol Assess. 28 (11): 1379-91). In addition to calculating continuous change from baseline to follow-up, a categorical outcome of clinically meaningful improvement, which was a decrease of 15 points or more from baseline to follow-up, was assessed (Marx et al. (2021) “Reliable and Clinically Significant Change in the Clinician-Administered PTSD Scale for DSM-5 and PTSD Checklist for DSM-5 among Male Veterans,” Psychol Assess (In Press)). When patients had multiple PCL measurements in the baseline or follow-up period, mean values were calculated.

Potential Confounders. Seven groups of potential confounders that could plausibly affect the relationship between treatment and outcome were measured (Table 7). Potential confounders spanned categories of HCV disease status, PTSD treatment history, PCL checklist availability, concurrent treatment, patient characteristics, health services use, and comorbid diagnoses (The Management of Posttraumatic Stress Disorder Work Group. VA/DOD CPG for the Management of PTSD and ASD. United States Departments of Veterans Affairs and Defense: Washington, DC: 2017).

### Table 7.

TABLE 7
Explanation of Covariates ^{a}
Hepatitis C Disease Status

| | |
|---|---|
| Chronicity | Days from HCV Diagnosis to Direct Acting Antiviral Medication |
| | Start ^{b} |
| Fibrosis | FIB-4 Score Rated as Mild, Moderate, or Advanced ^{c} |

PTSD Treatment History

| | |
|---|---|
| Chronicity | Days from PTSD Diagnosis to Direct Acting Antiviral Medication |
| | Start ^{b} |
| Evidence-Based | Receipt of at least 12 weeks of continuous treatment with |
| Antidepressants | medications recommended by VA clinical practice guidelines for |
| | PTSD. ^{d} |
| Evidence-Based | Receipt of at least 8 sessions prolonged exposure or cognitive |
| Psychotherapy | processing therapy over the course of one year. ^{e} |

PTSD Checklist Availability

| | |
|---|---|
| Timing | Days between baseline and follow-up PCL scores relative to |
| | medication start and stop dates. |
| Severity | Baseline score and whether the baseline score met diagnostic |
| | threshold. ^{g} |

Concurrent Treatments

| | |
|---|---|
| Evidence-Based | Number of weeks of treatment with mediations recommended |
| Antidepressants | medications recommended by VA clinical practice guidelines for |
| | PTSD. ^{d} |
| Evidence-Based | Number of sessions of prolonged exposure or cognitive processing |
| Psychotherapy | therapy. ^{e} |
| Other Medications | Categorical receipt of other antidepressants, anticonvulsants, |
| | sedative hypnotics, opioids, atypical antipsychotics, prazosin, as |
| | well as medications for alcohol use disorder and opioid agonist |
| | treatments. |

Patient Characteristics at Baseline

| | |
|---|---|
| Age | Continuous. |
| Sex | Categorical male or female |
| Race | Categorical Black Non-Hispanic, Hispanic, White Non-Hispanic, |
| | Other |
| Military Exposures | Combat, Sexual Trauma, Service-Connected Disability |

VA Health Service use Characteristics in the Year Preceding Baseline

| | |
|---|---|
| Outpatient visits | e.g., visits to specialized PTSD clinics or to primary care clinics |
| Acute psychiatric | Emergency department and urgent care visits for psychiatric |
| care use | indications, psychiatric hospitalizations |
| Residential treatment | Stays in residential PTSD or substance abuse programs |

Diagnoses in the Two Years Preceding Baseline

| | |
|---|---|
| Psychiatric | Number of non-PTSD Diagnoses and three most common |
| Comorbidities | diagnoses. ^{h} |
| Medical | Number of non-HCV Diagnoses and three most common diagnoses. ^{h} |
| Comorbidities | |

^{a} VA = US Department of Veterans Affairs, HCV = Hepatitis C Virus, PTSD = Posttraumatic Stress Disorder PCL = PTSD Checklist, PE = Prolonged Exposure, CPT = Cognitive Processing Therapy
^{b} Variable was assessed with a look-back period to Oct. 1, 1999.
^{c} FIB-4 grades level of hepatic fibrosis based on age, platelet counts, and aminotransferase levels (Sterling et al. (2006) Hepatology. 43(6): 1317-25; Vallet-Pichard et al. (2007) Hepatology. 46(1): 32-6).
^{d} Including fluoxetine, paroxetine, sertraline, and venlafaxine (The Management of Posttraumatic Stress Disorder Work Group. VA/DoD CPG for the Management of PTSD and ASD. United States Departments of Veterans Affairs and Defense: Washington, DC; 2017).
^{e} Measured using a natural language processing algorithm that classifies psychotherapy note text (Maguen et al. (2018) Adm Policy Ment Health. 45(4): 519-29).
^{f} PCL-IV scores were converted to PCL-5 scoring using a validated crosswalk (Moshier et al. (2019) J Trauma Stress 32(5): 799-805).
^{g} Cutoff of 31 based on optimal diagnostic efficiency (Bovin et al. (2016) Psychol Assess. 28(11): 1379-91).
^{h} Using a previously published index of mental and physical comorbidities adapted for VA medical record data (Shiner et al. (2021) J Behav Med. 44(4): 492-506).

Analysis. The first step in the analysis was to balance confounders across the DAA medication exposure groups. Consistent with modern epidemiologic principles of confounder identification (Lash T L, VanderWeele T J, Haneuse S, Rothman K J. Modern Epidemiology, Fourth Edition. Wolters Kluwer: New York: 2020), covariates with a standardized mean difference (SMD) of greater than or equal to 0.2 were considered as confounders for the univariate association with clinically meaningful improvement and for the bivariate relationship between exposure to one DAA versus each of the other DAAs. After identification of confounders, propensity scores representing the probability that a particular trial would be of each DAA combination were calculated (Stuart E A. (2010) Statistical Science 25 (1): 1-21). Propensity scores with multinomial logistic regression were estimated using generalized booster effects (McCaffrey et al. (2013) Statistics in medicine 32 (19): 3388-414), in which the dependent variable is an indicator for each of the three medications and confounders are the independent variables (Stuart E A. (2010) Statistical Science 25 (1): 1-21: McCaffrey et al. (2013) Statistics in medicine 32 (19): 3388-414). Weights were estimated based on the population average treatment effect model.

The second step in the analysis was to compare continuous and categorical PCL outcomes among the three DAA combinations with weighted regression analyses, using DAA combination received as the independent variable. These weighted medication groups were defined by the inverse of the propensity scores and adjusted for covariates unbalanced at the SMD ≥0.2 level. For the continuous outcome of change in total PCL score, weighted linear regression analysis was used, whereby the coefficient of the variable tests the hypothesis that each of the three DAA combinations, coded as a multilevel categorical variable, has the same mean change from baseline to follow-up. For the categorical outcome of clinically meaningful improvement, weighted logistic regression analysis was used, whereby the coefficient of the variable tests the hypothesis that each of the three DAA combinations results in the same percentage of patients achieving clinically meaningful improvement. To examine the possibility that any effect observed is due to HCV cure, all analyses were repeated excluding patients without evidence of HCV cure. If HCV cure explains the effect of DAAs on PTSD symptoms, a stronger association between medication and PCL improvement would be expected in the group containing only patients who were cured. Data management was performed in SAS version 9.4 (SAS Institute) and statistical modeling in R version 4.0.2 (R core team).

b. Results

A total of 253 patients met the inclusion criteria, including 54 who received GLE/PIB, 145 who received LDV/SOF, and 54 who received SOF/VEL (Table 8). There were several differences between patients across groups (Table 9). Particularly, those who received LDV/SOF had a longer duration of HCV illness, higher levels of fibrosis, greater concurrent use of sedative hypnotics and prazosin, less concurrent use of medications for alcohol use disorder, older age, more primary care visits, fewer emergency department visits for mental health indications, fewer admissions to acute inpatient and residential settings, lower rates of substance use disorder diagnoses, and lower rates of non-HCV liver disease diagnoses compared to patients in the other groups. Patients in the GLE/PIB group were less likely to be women. Additionally, there were several differences in the timing of PCL scores relative to DAA trials, with the shortest time from baseline PCL to DAA start in the GLE/PIB group and the shortest time from follow-up PCL to DAA end in the SOF/VEL group. Mean baseline PCL scores were very similar and in the severe range at approximately 50 across groups, although patients in the SOF/VEL group were more likely to have a baseline score of ≥31.

### Table 8.

TABLE 8
Patient Selection for Analysis of Direct-Acting Antivirals
(DAAs) for Posttraumatic Stress Disorder (PTSD) ^{a}

| Question | | Answer |
|---|---|---|
| A | How many Department of Veterans Affairs (VA) patients had any | 2,098,389 |
| | diagnosis of PTSD between FY2000 and FY2019? | |
| B | How many patients from (A) also had a diagnosis of Hepatitis C | 146,915 |
| | Virus (HCV)? | |
| C | How many patients from (B) received at least one prescription of a | 47,220 |
| | DAA for HCV? | |
| D | How many patients from (C) received at least one prescription for | 36,347 |
| | GLE/PIB, LDV/SOF, or SOF/VEL? | |
| E | How many prescriptions from (D) followed a 6-month washout of | 37,825 |
| | that medication? | |
| F | How many prescriptions from (E) were continued for at least 56 | 26,443 |
| | days? | |
| G | How many starts from (F) had a PTSD Checklist (PCL) within 90 | 1,063 |
| | days prior to start. | |
| H | How many starts from (G) had a PCL within 90 days after the 28-day | 253 |
| | point? | |

Detailed Information from (H): Person Trials from All Potential Patients

| GLE/PIB | LDV/SOF | SOF/VEL |
|---|---|---|
| 54 | 145 | 54 |

^{a} GLE/PIB = Glecaprevir/Pibrentasvir, LDV/SOF = Ledipasvir/Sofosbuvir, SOF/VEL = Sofosbuvir/Velpatasvir

### Table 9.

TABLE 9
Patient and Clinical Characteristics ^{a}

| | GLE/PIB | | LDV/SOF | | SOF/VEL | |
|---|---|---|---|---|---|---|
| | (N = 54) | | (N = 145) | | (N = 54) | |
| Drug Combination | n | % | n | % | n | % |

Hepatitis Disease Status

| | | | |
|---|---|---|---|
| Thousands of Days Since HCV | 1.8 ± 1.9 | 2.3 ± 2.1 | 1.4 ± 1.9 |
| Diagnosis (M ± SD) | | | |

Fibrosis (FIB-4 Score)

| | | | | | | |
|---|---|---|---|---|---|---|
| Mild (<1.45) | 32 | 59.3 | 75 | 51.7 | 37 | 68.5 |
| Moderate (≥1.45 and ≤3.25) | 18 | 33.3 | 61 | 42.1 | 13 | 24.1 |
| Advanced (>3.25) | 4 | 7.4 | 9 | 6.2 | 4 | 7.4 |
| HCV Cure following DAA Treatment ^{b} | 50 | 92.6 | 137 | 94.5 | 47 | 87.0 |

m

| | | | |
|---|---|---|---|
| Thousands of Days since PTSD Diagnosis | 1.7 ± 1.7 | 2.0 ± 2.0 | 1.7 ± 1.8 |
| Number of Prior EBA Trials (M ± SD) | 0.5 ± 0.8 | 0.4 ± 0.8 | 0.4 ± 0.7 |
| Number of Prior EBP Trials (M ± SD) | 0.2 ± 0.4 | 0.2 ± 0.4 | 0.2 ± 0.4 |

PCL Timing, Version, and Severity

| | | | | | | |
|---|---|---|---|---|---|---|
| Days from DAA Start to Earliest | 26.9 ± 24.8 | | 38.7 ± 26.3 | | 32.1 ± 27.7 | |
| Baseline PCL (M ± SD) | | | | | | |
| Days from Baseline PCL to Follow- | 110.4 ± 41.7 | | 118.7 ± 37.7 | | 113.4 ± 42.2 | |
| Up PCL (M ± SD) | | | | | | |
| Days from DAA End to Follow-Up | 16.1 ± 32.6 | | 6.7 ± 28.8 | | 1.2 ± 32.0 | |
| PCL (M ± SD) | | | | | | |
| Baseline PCL (M ± SD) | 48.4 ± 16.3 | | 49.1 ± 15.9 | | 50.9 ± 16.1 | |
| Baseline PCL Score ≥31 | 45 | 83.3 | 122 | 84.1 | 49 | 90.7 |

Concurrent Treatment

| | | | | | | |
|---|---|---|---|---|---|---|
| Sessions of EBP for PTSD (M ± SD) | 4.3 ± 6.1 | | 3.2 ± 5.1 | | 3.5 ± 5.4 | |
| Weeks of EBA for PTSD (M ± SD) | 4.8 ± 6.7 | | 4.9 ± 7.1 | | 3.3 ± 5.5 | |
| Any Non-EBA Antidepressant | 28 | 51.9 | 104 | 71.7 | 37 | 68.5 |
| Any Anticonvulsant | 7 | 13.0 | 21 | 14.5 | 9 | 16.7 |
| Any Sedative/Hypnotics | SL | SL | 30 | 20.7 | SL | SL |
| Any Opioid | 15 | 27.8 | 50 | 34.5 | 9 | 16.7 |
| Any Atypical Antipsychotic | 11 | 20.4 | 38 | 26.2 | 15 | 27.8 |
| Any Prazosin | 15 | 27.8 | 65 | 44.8 | 16 | 29.6 |
| Any FDA Approved Medications for AUD | 10 | 18.5 | 10 | 6.9 | 8 | 14.8 |
| Any Opioid Agonist Therapy | 14 | 25.9 | 21 | 14.5 | 9 | 16.7 |

Patient Characteristics at Baseline

| | | | | | | |
|---|---|---|---|---|---|---|
| Age (M ± SD) | 48.1 ± 13.8 | | 50.9 ± 14.0 | | 43.8 ± 13.2 | |
| Women | SL | SL | 13 | 9.0 | 6 | 11.1 |
| Married | 13 | 24.1 | 42 | 29.0 | 10 | 18.5 |
| Rural | 12 | 22.2 | 41 | 28.3 | 11 | 20.4 |
| White Non-Hispanic | 39 | 72.2 | 90 | 62.1 | 40 | 74.1 |
| Black Non-Hispanic | 12 | 22.2 | 44 | 30.3 | 9 | 16.7 |
| Hispanic | SL | SL | 10 | 6.9 | 3 | 5.6 |
| Other Racial or Ethnic Group | SL | SL | SL | SL | SL | SL |
| Combat Exposure | 21 | 38.9 | 67 | 46.2 | 23 | 42.6 |
| Sexual Trauma while in Military | 12 | 22.2 | 34 | 23.4 | 14 | 25.9 |
| VA Disability Level 70% or Greater | 19 | 35.2 | 63 | 43.4 | 27 | 50.0 |

Service Use Characteristics in the 1 Year Preceding Baseline

| | | | |
|---|---|---|---|
| Number of PTSD Outpatient | 3.9 ± 7.0 | 6.8 ± 12.1 | 7.3 ± 14.5 |
| Clinical Team Visits (M ± SD) | | | |
| Number of Outpatient Mental | 58.5 ± 46.2 | 51.2 ± 41.3 | 64.7 ± 49.9 |
| Health Visits (M ± SD) | | | |
| Number of Outpatient Substance | 22.6 ± 22.6 | 17.8 ± 23.8 | 23.0 ± 26.0 |
| Abuse Visits (M ± SD) | | | |
| Number of Outpatient Primary Care | 5.4 ± 4.7 | 9.4 ± 7.3 | 6.8 ± 6.1 |
| Visits (M ± SD) | | | |
| Number of Outpatient Specialty | 3.0 ± 3.8 | 3.4 ± 2.6 | 3.8 ± 3.4 |
| Medical Visits (M ± SD) | | | |
| Number of ED Visits for Psychiatric | 1.3 ± 1.7 | 0.6 ± 1.3 | 1.6 ± 2.4 |
| Indication (M ± SD) | | | |
| Days of Acute Inpatient Mental | 9.2 ± 18.5 | 3.6 ± 12.7 | 9.4 ± 18.8 |
| Health (M ± SD) | | | |
| Days of Residential PTSD | 4.6 ± 19.5 | 3.1 ± 17.6 | 6.8 ± 30.6 |
| Treatment (M ± SD) | | | |
| Days Residential Substance Abuse | 35.6 ± 59.5 | 7.4 ± 25.7 | 17.2 ± 40.2 |
| Treatment (M ± SD) | | | |

Comorbidities in the 2 Years Preceding Baseline

| | | | | | | |
|---|---|---|---|---|---|---|
| Number of Non-PTSD MH | 3.8 ± 2.0 | | 3.2 ± 1.6 | | 3.5 ± 1.7 | |
| Comorbidities (M ± SD) | | | | | | |
| Substance Use Disorders | SH | SH | 114 | 78.6 | 50 | 92.6 |
| Depressive Disorders | 43 | 79.6 | 109 | 75.2 | 41 | 75.9 |
| Anxiety Disorders | 29 | 53.7 | 58 | 40.0 | 24 | 44.4 |
| Number of Non-PTSD PH | 2.3 ± 1.7 | | 2.3 ± 1.6 | | 2.1 ± 1.9 | |
| Comorbidities (M ± SD) | | | | | | |
| Non-HCV Liver Disease | 50 | 92.6 | 108 | 74.5 | 48 | 88.9 |
| Uncomplicated Hypertension | 22 | 40.7 | 62 | 42.8 | 18 | 33.3 |
| Obesity | 4 | 7.4 | 23 | 15.9 | 9 | 16.7 |

^{a} DAA = Direct-Acting Antiviral; PTSD = posttraumatic stress disorder, FY = Fiscal Year; VA = Department of Veterans Affairs; HCV = Hepatitis C Virus; EBA = Evidence-Based Antidepressant; EBP = Evidence-Based Psychotherapy PCL = PTSD Checklist; MH = Mental Health; PH = Physical Health; SL = suppressed low: cell is suppressed due to less than 3 patients; SH = suppressed high: cell is suppressed due to all but fewer than 3 patients
^{b} This variable was considered a potential moderator rather than a potential confounder.

Among the potential confounders, 14 were associated with both the outcome and the exposure and thus considered to be confounders (Table 10). Two of the confounders, sedative-hypnotic use and substance use disorder diagnoses, were insufficiently represented in one or more exposure groups and were not included in the propensity score model; this required creating weights based on one or two patients, which prevented the models from converging. Of the remaining 12 covariates, five remained imbalanced at the SMD>0.2 level after IPTW weighting (Table 11). The five unbalanced covariates, including concurrent prescription of opioids, concurrent prescription of prazosin, the number of primary care visits in the year preceding baseline, the number of emergency department visits for psychiatric indications in the year preceding baseline, and non-HCV liver disease diagnoses, were maintained as covariates in all weighted outcomes models.

### Table 10.

TABLE 10
Assessment of Potential Confounding, as Measured
by Standardized Mean Difference ^{a b}

| | Association | | | |
|---|---|---|---|---|
| | | Exposure | | |
| | Outcome | GLE/PIB | GLE/PIB | SOF/VEL |
| | 15-Point | versus | versus | versus |
| Variable | Improvement | LDV/SOF | SOF/VEL | LDV/SOF |

Hepatitis Disease Status

| | | | | |
|---|---|---|---|---|
| Thousands of Days Since HCV | 0.01 | 0.26 | 0.18 | 0.43 |
| Diagnosis | | | | |

Fibrosis (FIB-4 Score)

| | | | | |
|---|---|---|---|---|
| Mild (<1.45) | 0.29 | 0.15 | 0.19 | 0.35 |
| Moderate (≥1.45 and ≤3.25) | 0.28 | 0.18 | 0.21 | 0.39 |
| Advanced (>3.25) | 0.04 | 0.05 | 0.00 | 0.05 |

PTSD Treatment History

| | | | | |
|---|---|---|---|---|
| Thousands of Days since | 0.08 | 0.14 | 0.02 | 0.15 |
| PTSD Diagnosis | | | | |
| Number of Prior EBA Trials | 0.20 | 0.09 | 0.22 | 0.12 |
| Number of Prior EBP Trials | 0.07 | 0.05 | 0.05 | 0.00 |

PCL Timing, Version, and Severity

| | | | | |
|---|---|---|---|---|
| Days from DAA Start to | 0.16 | 0.46 | 0.20 | 0.25 |
| Earliest Baseline PCL | | | | |
| Days from Baseline PCL to | 0.22 | 0.21 | 0.07 | 0.13 |
| Follow-Up PCL | | | | |
| Days from DAA End to | 0.09 | 0.31 | 0.54 | 0.26 |
| Follow-Up PCL | | | | |
| Baseline PCL | 0.50 | 0.04 | 0.15 | 0.11 |
| Baseline PCL Score ≥31 | 0.37 | 0.02 | 0.22 | 0.20 |

Concurrent Treatment

| | | | | |
|---|---|---|---|---|
| Sessions of EBP for PTSD | 0.25 | 0.19 | 0.14 | 0.05 |
| Weeks of EBA for PTSD | 0.08 | 0.01 | 0.24 | 0.25 |
| Any Non-EBA Antidepressant | 0.10 | 0.42 | 0.35 | 0.07 |
| Any Anticonvulsant | 0.30 | 0.04 | 0.10 | 0.06 |
| Any Sedative/Hypnotics | 0.40 | 0.54 | 0.00 | 0.54 |
| Any Opioid | 0.26 | 0.15 | 0.27 | 0.42 |
| Any Atypical Antipsychotic | 0.09 | 0.14 | 0.17 | 0.04 |
| Any Prazosin | 0.23 | 0.36 | 0.04 | 0.32 |
| Any FDA Approved | 0.28 | 0.35 | 0.10 | 0.26 |
| Medications for AUD | | | | |
| Any Opioid Agonist Therapy | 0.09 | 0.29 | 0.23 | 0.06 |

Patient Characteristics at Baseline

| | | | | |
|---|---|---|---|---|
| Age | 0.09 | 0.20 | 0.32 | 0.52 |
| Women | 0.19 | 0.32 | 0.38 | 0.07 |
| Married | 0.04 | 0.11 | 0.14 | 0.25 |
| Rural | 0.24 | 0.14 | 0.05 | 0.19 |
| White Non-Hispanic | 0.05 | 0.22 | 0.04 | 0.26 |
| Black Non-Hispanic | 0.04 | 0.19 | 0.14 | 0.33 |
| Hispanic | 0.16 | 0.25 | 0.20 | 0.06 |
| Other Racial or Ethnic Group | 0.05 | 0.21 | 0.00 | 0.21 |
| Combat Exposure | 0.02 | 0.15 | 0.08 | 0.07 |
| Sexual Trauma while in | 0.14 | 0.03 | 0.09 | 0.06 |
| Military | | | | |
| VA Disability Level 70% or | 0.12 | 0.17 | 0.30 | 0.13 |
| Greater | | | | |

Service Use Characteristics in the 1 Year Preceding Baseline

| | | | | |
|---|---|---|---|---|
| Number of PTSD Outpatient | 0.25 | 0.30 | 0.30 | 0.04 |
| Clinical Team Visits | | | | |
| Number of Outpatient Mental | 0.07 | 0.17 | 0.13 | 0.29 |
| Health Visits | | | | |
| Number of Outpatient | 0.10 | 0.21 | 0.02 | 0.21 |
| Substance Abuse Visits | | | | |
| Number of Outpatient Primary | 0.33 | 0.65 | 0.26 | 0.38 |
| Care Visits | | | | |
| Number of Outpatient | 0.10 | 0.12 | 0.22 | 0.14 |
| Specialty Medical Visits | | | | |
| Number of ED Visits for | 0.25 | 0.47 | 0.18 | 0.56 |
| Psychiatric Indication | | | | |
| Days of Acute Inpatient Mental | 0.15 | 0.35 | 0.02 | 0.37 |
| Health | | | | |
| Days of Residential PTSD | 0.07 | 0.08 | 0.09 | 0.15 |
| Treatment | | | | |
| Days Residential Substance | 0.03 | 0.62 | 0.36 | 0.29 |
| Abuse Treatment | | | | |

Comorbidities in the 2 Years Preceding Baseline

| | | | | |
|---|---|---|---|---|
| Number of Non-PTSD MH | 0.08 | 0.37 | 0.17 | 0.21 |
| Comorbidities | | | | |
| Substance Use Disorders | 0.29 | 0.64 | 0.27 | 0.41 |
| Depressive Disorders | 0.10 | 0.11 | 0.09 | 0.02 |
| Anxiety Disorders | 0.14 | 0.28 | 0.19 | 0.09 |
| Number of Non-PTSD PH | 0.02 | 0.04 | 0.11 | 0.16 |
| Comorbidities | | | | |
| Non-HCV Liver Disease | 0.31 | 0.50 | 0.13 | 0.38 |
| Uncomplicated Hypertension | 0.17 | 0.04 | 0.15 | 0.20 |
| Obesity | 0.08 | 0.27 | 0.29 | 0.02 |

^{a} DAA = Direct-Acting Antiviral; PTSD = posttraumatic stress disorder, FY = Fiscal Year; VA = Department of Veterans Affairs; HCV = Hepatitis C Virus; EBA = Evidence-Based Antidepressant; EBP = Evidence-Based Psychotherapy PCL = PTSD Checklist; MH = Mental Health; PH = Physical Health
^{b} Covariates associated with both the outcome and exposure (SMD ≥ 0.2) are indicated by the gray rows.

### Table 11.

TABLE 11
Post-Weighting Balance of Confounders ^{a}

| | GLE/PIB (N ± 54) | | LDV/SOF (N ± 145) | | SOF/VEL (N ± 54) | |
|---|---|---|---|---|---|---|
| Drug Combination | n | % | n | % | n | % |
| Mild Fibrosis | 32 | 54.9 | 75 | 55.3 | 37 | 59.9 |
| Moderate Fibrosis | 18 | 32.8 | 61 | 38.0 | 13 | 32.2 |
| Number of Prior EBA | 0.4 ± 0.8 | | 0.4 ± 0.8 | | 0.4 ± 0.7 | |
| Trials (M ± SD) | | | | | | |
| Days from Baseline PCL to | 114.2 ± 38.2 | | 117.2 ± 37.2 | | 114.5 ± 39.2 | |
| Follow-Up PCL (M ± SD) | | | | | | |
| Baseline PCL Score ≥31 | 45 | 84.3 | 122 | 85.1 | 49 | 90.5 |
| Any Concurrent Opioid ^{b} | 15 | 28.1 | 50 | 31.3 | 9 | 22.2 |
| Any Concurrent Prazosin ^{b} | 15 | 30.0 | 65 | 40.2 | 16 | 25.3 |
| Any Concurrent FDA | 10 | 12.4 | 10 | 7.0 | 8 | 11.6 |
| Approved Medications for | | | | | | |
| AUD | | | | | | |
| Prior Year PTSD Outpatient | 4.4 ± 7.0 | | 6.2 ± 11.4 | | 5.8 ± 12.3 | |
| Clinical Team Visits | | | | | | |
| (M ± SD) | | | | | | |
| Prior Year Outpatient | 6.1 ± 4.6 | | 8.4 ± 7.1 | | 7.8 ± 7.0 | |
| Primary Care Visits | | | | | | |
| (M ± SD) ^{b} | | | | | | |
| Prior Year ED Visits for | 1.0 ± 1.6 | | 0.9 ± 1.5 | | 1.3 ± 2.1 | |
| Psychiatric Indication | | | | | | |
| (M ± SD) ^{b} | | | | | | |
| Non-HCV Liver Disease | 50 | 86.2 | 108 | 79.3 | 48 | 89.7 |
| Diagnosis in Prior Two | | | | | | |
| Years ^{b} | | | | | | |

^{a} DAA = Direct-Acting Antiviral; PTSD = posttraumatic stress disorder, FY = Fiscal Year; VA = Department of Veterans Affairs; HCV = Hepatitis C Virus; EBA = Evidence-Based Antidepressant; EBP = Evidence-Based Psychotherapy PCL = PTSD Checklist; MH = Mental Health; PH = Physical Health
^{b} Variable was not balanced (SMD < 0.2) after weighting and was retained as a covariate in outcomes models.

In the unweighted analyses, there were large overall differences in both mean change in PCL score and the percentage of patients who improved by 15 points or more (Table 12 Top Panel). The largest mean improvement in PCL score was 12.9 points (SD=14.3) for the GLE/PIB group, and the smallest mean improvement in PCL score was 3.1 points (SD=14.1) for the LDV/SOF group. This translated to a mean difference of 9.79 points between the GLE/PIB and LDV/SOF groups (95% CI: 5.33, 14.25). The difference was slightly smaller in the propensity score weighted model at 7.34 points (95% CI: 1.05, 13.63). Similarly, the unweighted proportion of patients improving by 15 points or more on the PCL was 40.7% for the GLE/PIB group and 17.9% for the LDV/SOF group. This translated to an odds ratio of 2.27 (95% CI: 1.42, 3.65). The effect was similar in weighted models at 2.17 (95% CI: 0.93, 5.06). While SOF/VEL was superior to LDV/SOF in unweighted analyses (MD=6.01; 95% CI: 1.47, 10.54), the effect was greatly diminished in the propensity score weighted model (MD=2.95; 95% CI: −6.33, 12.23). Similarly, the advantage of SOF/VEL over LDV/SOF in the proportion of patients improving by 15 points or more diminished considerably when moving from the unweighted model (OR-1.97; 95% CI: 1.18, 3.28) to the propensity score weighted model (OR=1.54; 95% CI: 0.45, 5.24). The effect of GLE/PIB relative to LDV/SOF was somewhat weaker in magnitude in our analyses excluding patients without evidence of HCV cure (Table 12 Bottom Panel), arguing against the idea that HCV cure is required for a PTSD treatment effect.

### Table 12.

TABLE 12
Baseline PCL and Outcomes ^{a}
All Patients

| GLE/PIB | LDV/SOF | SOF/VEL | GLE/PIB versus | | GLE/PIB versus | | SOF/VEL versus | |
|---|---|---|---|---|---|---|---|---|
| N = 54 | N = 145 | N = 54 | LDV/SOF | | SOF/VEL | | LDV/SOF | |
| M ± SD | M ± SD | M ± SD | MD | 95% CI | MD | 95% CI | MD | 95% CI |

Baseline PCL

| | | | | | | | | | |
|---|---|---|---|---|---|---|---|---|---|
| Unweighted | 48.4 ± 16.3 | 49.1 ± 15.9 | 50.9 ± 16.1 | 0.67 | −4.39, 5.73 | 2.47 | −3.63, 8.57 | −1.80 | −6.81, 3.21 |
| IPTW | 44.1 ± 20.8 | 47.2 ± 49.1 | 48.0 ± 32.2 | 3.10 | −2.64, 8.84 | 3.89 | −2.67, 10.46 | −0.80 | −9.52, 7.92 |

Change in PCL

| | | | | | | | | | |
|---|---|---|---|---|---|---|---|---|---|
| Unweighted | −12.9 ± 14.3 | −3.1 ± 14.1 | −9.2 ± 14.7 | 9.79 | 5.33, 14.25 | 3.78 | −1.68, 9.25 | 6.01 | 1.47, 10.54 |
| IPTW | −14.9 ± 33.2 | −7.5 ± 66.8 | −10.5 ± 41.9 | 7.34 | 1.05, 13.63 | 4.39 | −2.43, 11.21 | 2.95 | −6.33, 12.23 |

Clinically Meaningful Improvement

| | n | % | n | % | n | % | OR | 95% CI | OR | 95% CI | OR | 95% CI |
|---|---|---|---|---|---|---|---|---|---|---|---|---|
| Unweighted | 22 | 40.7 | 26 | 17.9 | 19 | 35.2 | 2.27 | 1.42, 3.65 | 1.16 | 0.71, 1.88 | 1.97 | 1.18, 3.28 |
| IPTW | 22 | 43.6 | 26 | 26.3 | 19 | 30.3 | 2.17 | 0.93, 5.06 | 1.41 | 0.58, 3.42 | 1.54 | 0.45, 5.24 |

Excluding Patients without Evidence of Hepatitis C Virus Cure

| GLE/PIB | LDV/SOF | SOF/VEL | GLE/PIB versus | | GLE/PIB versus | | SOF/VEL versus | |
|---|---|---|---|---|---|---|---|---|
| N = 50 | N = 137 | N = 47 | LDV/SOF | | SOF/VEL | | LDV/SOF | |
| M ± SD | M ± SD | M ± SD | MD | 95% CI | MD | 95% CI | MD | 95% CI |

Baseline PCL

| | | | | | | | | | |
|---|---|---|---|---|---|---|---|---|---|
| Unweighted | 48.3 ± 16.3 | 48.7 ± 16.0 | 51.5 ± 14.5 | 0.40 | −4.86, 5.66 | 3.20 | −2.95, 9.35 | −2.80 | −7.74, 2.14 |
| IPTW | 43.8 ± 23.1 | 47.2 ± 53.3 | 48.1 ± 34.5 | 3.44 | −2.67, 9.55 | 4.29 | −2.54, 11.12 | −0.85 | −10.02, 8.31 |

Change in PCL

| | | | | | | | | | |
|---|---|---|---|---|---|---|---|---|---|
| Unweighted | −12.6 ± 13.6 | −2.4 ± 13.7 | −8.8 ± 13.4 | 10.21 | 5.80, 14.62 | 3.87 | −1.50, 9.24 | 6.34 | 1.87, 10.81 |
| IPTW | −11.9 ± 26.5 | −5.7 ± 56.3 | −7.6 ± 35.8 | 6.17 | 0.34, 12.01 | 4.26 | −2.17, 10.69 | 1.92 | −6.77, 10.60 |

Clinically Meaningful Improvement

| | n | % | n | % | n | % | OR | 95% CI | OR | 95% CI | OR | 95% CI |
|---|---|---|---|---|---|---|---|---|---|---|---|---|
| Unweighted | 20 | 40.0 | 23 | 16.8 | 15 | 31.9 | 2.38 | 1.44, 3.94 | 1.25 | 0.73, 2.15 | 1.90 | 1.08, 3.32 |
| IPTW | 20 | 30.1 | 23 | 19.0 | 15 | 21.6 | 1.83 | 0.77, 4.36 | 1.57 | 0.62, 3.98 | 1.17 | 0.33, 4.18 |

^{a} MD = Mean Difference, OR = Odds Ratio, CI = Confidence Interval, PCL = PTSD Checklist, M = Mean, SD = Standard Deviation, IPTW = Inverse Propensity of Treatment Weighting

c. Discussion

Though almost all patients in the study were cured of HCV, those receiving certain DAAs experienced greater PTSD symptom improvement. Consistent with the hypothesis, patients in both the GLE/PIB group and the SOF/VEL group had superior outcomes to patients in the LDV/SOF group in unadjusted analyses. However, GLE/PIB appears to be more strongly associated with PTSD symptom improvement in adjusted analyses. Without wishing to be bound by theory, this suggests that among DAAs commonly used in the VA, GLE/PIB is the strongest candidate for continued research as a potential PTSD treatment. In the current study, patients receiving GLE/PIB improved by a mean of approximately 13 points on the PCL. Other recent analyses using the same VA PCL dataset indicate that this level of improvement is larger than typically observed in routine practice for evidence-based treatments recommended by the clinical practice guideline for PTSD from the VA and Department of Defense (The Management of Posttraumatic Stress Disorder Work Group. VA/DoD CPG for the Management of PTSD and ASD. United States Departments of Veterans Affairs and Defense: Washington, DC: 2017). First, in a study of almost 7,000 patients receiving adequately dosed antidepressants, PTSD symptoms improved by only 5-6 points over 12 weeks (Shiner et al. (2021) J Clin Psychiatry. 82 (6)). Second, in over 100,000 patients receiving ≥8 sessions of prolonged exposure or cognitive processing therapy over ≤14 weeks, patients improved by a mean of 8-10 points (Shiner et al. (2021) “Improvements to PTSD quality metrics with natural language processing.” J Eval Clin Pract.).

When considering known mechanisms of action for the three DAA combinations that were studied, all contain NS5A protein inhibitors (ledipasvir, pibrentasvir, and velpatasvir). Two of the DAAs contain a NS5B polymerase inhibitor (sofosbuvir). The most promising agent, GLE/PIB, is unique in that it contains a NS3/4A protease inhibitor (glecaprevir). HCV NS3 protein has been found to cross the blood brain barrier (BBB), activating microglia, resulting in the release of pro-inflammatory cytokines, which has been linked to neurological impairment (Wilkinson et al. (2010) Gut. 59 (10): 1394-400; Adinolfi et al. (2015) World J Gastroenterol. 21 (8): 2269-80). By inhibiting the HCV NS3/4A protease, glecaprevir prevents HCV viral replication (Zajac et al. (2019) Eur J Med Chem. 165:225-49). Given the FDA clinical review of glecaprevir notes poor BBB permeability (Stabinski L, Hodowenec A. NDA 209394 Clinical Review: Mavyret (glecaprevir and pibrentasvir). United States Food and Drug Administration: Washington, DC: 2015), the medication could potentially block HCV from passing the BBB altogether to activate microglia. While it is unclear how this mechanism would target PTSD symptoms (Yehuda et al. (2015) Nat Rev Dis Primers. 1:15057), many of the pro-inflammatory cytokines that are released in the presence of HCV infection are consistent with those identified as biomarkers for PTSD, which lends to systemic inflammation in PTSD pathophysiology (Bhatt et al. (2020) Nature Communications. 11 (1): 2360; Kim et al. (2020) Antioxidants (Basel). 9 (2): 107: Hori and Kim (2019) Psychiatry and Clinical Neurosciences. 73 (4): 143-53). While this would explain GLE/PIB's effect on PTSD symptoms following HCV clearance, it would not necessarily explain why PTSD might improve in the absence of HCV infection. However, given current knowledge on NS3 and NS5 inhibitors (McGivern et al. (2015) Journal of Virology. 89 (10): 5362-70; Rivas-Estilla et al. (2002) Journal of Virology. 76 (21): 10637-53), it is plausible that GLE/PIB is acting on a cellular as opposed to a viral level with off target PTSD effects via the inflammatory process.

3. Changes in Alcohol Consumption Following Direct-Acting Antiviral Treatment for Hepatitis C in VA Patients with Comorbid Alcohol Use Disorder and PTSD

Alcohol use disorder (AUD) is a common substance use disorder (SUD) in the United States (US), afflicting 5% of persons over age 12 and associated with significant disability (Tucker et al., 2020). Among the US population, an estimated 42% of people with AUD have a co-occurring post-traumatic stress disorder (PTSD) diagnosis (Pietrzak et al., 2011). Among those with PTSD, the prevalence of AUD ranges from 9.8% to 61.3% (Debell et al., 2014). The association of AUD and PTSD is especially strong among US military veterans. In this group, 63% of those with SUD have co-occurring PTSD, and over 75% of veterans with PTSD have co-occurring SUD (Seal, Cohen, Bertenthal, et al., 2011). There is a 4-fold increase in the odds of a comorbid AUD diagnosis for Iraq and Afghanistan Veterans with PTSD (Seal, Cohen, Waldrop, et al., 2011). One hypothesis for this high rate of co-occurrence is self-medication, where PTSD leads to drinking in order to alleviate trauma-related symptoms (Kwako et al., 2015: McLean et al., 2015). This problem may be compounded by limited access to evidence-based treatments for PTSD (Maguen et al., 2020: Shiner et al., 2018; Watkins et al., 2018).

Hepatitis C virus (HCV) is spread through blood contact (often via needle sharing), infecting the liver and resulting in acute illness, with nearly half of patients proceeding to develop chronic infection associated with cirrhosis and liver cancers (Memon & Memon, 2002: Westbrook & Dusheiko, 2014). HCV-infected liver cells are known to activate an immune cell protein called the NLRP3 inflammasome, which can lead to chronic systemic inflammation (Chen et al., 2014: Ramachandran et al., 2021). The role of hyperactive NLRP3 is well established in many diseases including various cancers, metabolic disorders, autoimmune disorders, and Alzheimer's disease (Fusco et al., 2020; Sharma & Kanneganti, 2021: Wang et al., 2020).

Chronic HCV is more prevalent in persons with AUD than in the general population (Marshall, 2021), and an estimated 44% of veterans with HCV have a history of AUD (Butt et al., 2007). AUD and HCV are common causes of chronic liver disease, and together they have a synergistic effect on its progression (Marshall, 2021). Prior to the advent of current direct-acting antiviral (DAA) treatment for HCV, research suggested that alcohol consumption was associated with a decreased likelihood of sustained virologic response (SVR) or HCV cure (Okazaki et al., 1994). Therefore, clinicians treating HCV would often delay therapy until patients were abstinent from alcohol (Okazaki et al., 1994). Thus, most of the available literature in this area focuses on the impact of alcohol consumption on HCV treatment efficacy, hepatic functioning and rates of hepatocellular carcinoma (Singal & Anand, 2007). Alcohol consumption negatively impacted the previous generation of treatment for HCV, pegylated interferon (Chang et al., 2005). Because of this history, the impact of alcohol consumption on SVR has been carefully monitored with the emergence of DAAs for HCV, and a growing literature now indicates that alcohol consumption has little to no impact on DAA efficacy (Tsui et al., 2016). Despite the growing evidence against withholding DAA treatment until abstinence from alcohol consumption, many payers—and consequently clinicians—continued to endorse this practice (Anand et al., 2006: Barua et al., 2015). In 2015, the Centers for Medicare & Medicaid Services (CMS) issued a notice to relax policies that required abstinence from alcohol and substances in order to obtain DAA treatment for HCV (Herink et al., 2021). In 2016, the Department of Veterans Affairs (VA) followed suit by updating Clinical Practice Guidelines to encourage HCV treatment providers to prescribe DAAs regardless of alcohol or substance use status (Owens et al., 2018). Additionally in 2016, the VA announced the expansion of treatment to all HCV patients regardless of liver disease stage, prior to which, limited resources prioritized HCV treatments to the sickest patients (VA, 2016).

Unexpected but highly promising epidemiological data suggest that two DAAs for HCV, sofosbuvir/velpatasvir (SOF/VEL), which was FDA approved in 2016 (Jackson & Everson, 2017), and glecaprevir/pibrentasvir (GLE/PIB which was FDA-approved in 2017, (Lamb, 2017), are associated with PTSD symptom improvement (Shiner, Forehand, et al., 2022). While all DAAs for HCV are associated with a greater than 90% SVR in VA patients (Belperio et al., 2017), there was no association with PTSD symptom improvement for a third commonly-prescribed DAA called ledipasvir/sofosbuvir (LDV/SOF) which was FDA-approved in 2014 (Kowdley et al., 2014). Subsequent analyses controlling for patient differences between agents indicated that GLE/PIB was the most strongly associated DAA with PTSD symptom improvement (Shiner, Huybrechts, et al., 2022). Although it was of interest to explore the mediating role of common comorbid psychiatric disorders such as AUD underlying the association of DAA treatment with PTSD symptom improvement, the sample size was too small for formal mediation analyses.

The biological mechanism by which DAAs could have psychiatric effects is unclear. However, emerging evidence has linked NLRP3 activation to various psychiatric disorders likely due to its role in neuroinflammation (çelik et al., 2022). It has been established that NLRP3 can be activated by pathogens, such as HCV, or environmental stressors, such has fear for one's life (Cheon et al., 2020). In animal models of PTSD and anxiety, NLRP3 activation has demonstrated association with dysregulation of fear memory and response (Dong et al., 2020). Furthermore, NLRP3 inhibition has shown improved anxiety (Yamanashi et al., 2020). Administration of alcohol in animal models has exhibited inhibition of NLRP3 (Hoyt et al., 2016), thereby supporting the PTSD/AUD self-medication hypothesis. Additionally, NLRP3 inhibition has demonstrated reduced alcohol consumption in mice (Lowe et al., 2020). Thus, theoretically, if a medication were to inhibit NLRP3 activity, it is plausible that it could simultaneously inhibit HCV replication, fear response, and alcohol consumption. While existing DAAs are only classified as acting on virus, there is some evidence of off-target effects (Giovannini et al., 2020).

The goal of this study was to identify individuals with comorbid PTSD, AUD, and HCV who completed a standardized assessment of alcohol consumption before and after DAA treatment and assess clinically meaningful change in alcohol consumption between the specific DAA treatments. To our knowledge, there has only been one study evaluating the impact of DAA treatment on alcohol consumption. A retrospective cohort study of VA patients receiving DAAs for HCV from 2013-2015 (prior to the FDA approval of GLE/PIB and SOF/VEL) found that the overall frequency of alcohol misuse declined from 9.1% to 7.7% following DAA treatment (Kim et al., 2020). Our study differs in several ways. First, we include the most recently approved DAAs for HCV, GLE/PIB and SOF/VEL, which were not included in the prior work and provide data following the directive discouraging the withholding of DAAs until alcohol abstinence. Second, we specifically focus on patients with diagnoses of AUD and comorbid PTSD with levels of alcohol consumption consistent with current AUD. In addition to the primary goal of understanding the impact of HCV treatment on alcohol use among a sample with co-occurring AUD and PTSD, we further compared DAA treatments with one another to determine if observations are tied to specific treatments, as has been observed for PTSD (Shiner, Huybrechts, et al., 2022), or if DAAs have an overall effect on change in AUD regardless of the specific treatment used.

a. Methods

Data Sources and Study Sample: To conduct the present study; a subgroup of the parent cohort of Veterans with PTSD that was constructed for the initial exploratory study was used (Shiner, Forehand, et al., 2022). Following the parent cohort construction, VA patients with comorbid diagnoses of PTSD, AUD, and HCV between Oct. 1, 1999 and Sep. 30, 2019 were identified using the VA Corporate Data Warehouse (CDW), a national repository of electronic medical record data (Price et al., 2015). International Classification of Diseases (ICD) Versions 9 and 10 codes were used to ascertain diagnoses of PTSD (ICD-9): 309.81: ICD-10: F43.1x), HCV (ICD-9:070.41, 070.44, 070.51, 070.54, 070.7, 070.70, 070.71; ICD-10: B17.1, B17.10, B17.11, B19.2, B19.20, B19.21, B18.2), and AUD (ICD-9:303.x, 305.0x: ICD-10: F10.x). Diagnostic information in the CDW was supplemented with linked Medicare files when available. The sample was restricted to patients who completed a course of DAA (see direct acting antiviral treatment, below). The sample was further restricted to patients who had standardized assessments of alcohol consumption at baseline and follow-up, as well as heavy alcohol use at baseline (see alcohol consumption measurement, below). Patients whose first AUD diagnosis occurred after DAA initiation were excluded to ensure that our study population contained only persons who clearly had active AUD at the time of DAA treatment. The Veterans Institutional Review Board of Northern New England approved this study.

Direct Acting Antiviral Treatment: Patients who received at least 56 continuous days of prescription drug fills for the DAA agents LDV/SOF, SOF/VEL, and GLE/PIB were identified. A validation study of pharmacy fill records for DAA agents in the VA CDW found that 98% (95% CI: 97%, 100%) of recorded DAA fills in the VA CDW were confirmed after clinician verification (Rentsch et al., 2018). Patients were included if their DAA trial and related alcohol consumption measurement (see below) was completed by February 2020.

Alcohol Consumption Measurement: Alcohol consumption was measured using the Alcohol Use Disorders Identification Test-Consumption (AUDIT-C) Module (Bush et al., 1998), a validated instrument administered annually as part of behavioral health screening in the VA (Shiner et al., 2014). A baseline AUDIT-C was required within year prior to DAA start and a follow-up AUDIT-C between one month and one year after DAA start. A baseline score of 8 or higher was required, indicating a level of alcohol consumption consistent with current dependence (Rubinsky et al., 2013). Clinically meaningful improvement was examined as a binary variable (yes/no), defined as a follow-up AUDIT-C score below the threshold (three points or less for men and two points or less for women) for alcohol misuse screening (Bradley et al., 2009). Continuous change in AUDIT-C score from baseline to follow-up was also examined. For patients who had multiple AUDIT-C scores in the baseline or follow-up period, the mean values were calculated for each period.

Covariates: Seven groups of covariates were measured (Table 13): HCV disease status, prior evidence-based PTSD treatment, timing of AUDIT-C scores relative to DAA initiation, concurrent mental health treatment, patient demographic characteristics at baseline, VA health services use in the year prior to DAA initiation, and comorbidities in the two years prior to DAA initiation.

### Table 13.

TABLE 13
Explanation of Covariates ^{a}
Hepatitis C Disease Status

| | |
|---|---|
| Chronicity | Days from HCV diagnosis to Direct Acting Antiviral |
| | medication start ^{b} |
| Fibrosis | FIB-4 score rated as mild, moderate, or advanced ^{c} |

Prior Evidence-Based PTSD Treatment

| | |
|---|---|
| Evidence-Based | Receipt of at least 12 weeks of continuous treatment with |
| Antidepressants | medications recommended by VA clinical practice guidelines |
| | for depression ^{d} |
| Evidence-Based | Receipt of at least 8 sessions prolonged exposure or cognitive |
| Psychotherapy | processing therapy over the course of one year ^{e} |

AUDIT-C Availability

| | |
|---|---|
| Chronicity | Days from alcohol use disorder diagnosis to Direct Acting |
| | Antiviral medication start ^{b} |
| Timing | Days between baseline and follow-up AUDIT-C scores relative |
| | to medication start and stop dates |

Concurrent Treatments

| | |
|---|---|
| Evidence-Based | Number of weeks of treatment with medications recommended |
| Antidepressants | by VA clinical practice guidelines for depression ^{d} |
| Evidence-Based | Number of sessions of prolonged exposure or cognitive |
| Psychotherapy | processing therapy ^{e} |
| Other Medications | Categorical receipt of other antidepressants, anticonvulsants, |
| | sedative hypnotics, opioids, atypical antipsychotics, prazosin, |
| | as well as medications for alcohol use disorder and opioid |
| | agonist treatments. |

Patient Characteristics at Baseline

| | |
|---|---|
| Age | Continuous |
| Sex | Categorical male or female |
| Race | Categorical Black Non-Hispanic, Hispanic, White Non- |
| | Hispanic, Other |
| Military Exposures | Combat, Sexual Trauma, Service-Connected Disability |

VA Health Service use Characteristics in the Year Preceding Baseline

| | |
|---|---|
| Outpatient visits | e.g., visits to specialized PTSD clinics or to primary care |
| | clinics |
| Acute psychiatric | Emergency department and urgent care visits for psychiatric |
| care use | indications, psychiatric hospitalizations |
| Residential treatment | Stays in residential PTSD or substance abuse programs |

Diagnoses in the Two Years Preceding Baseline

| | |
|---|---|
| Psychiatric | Number of non-alcohol use disorder diagnoses and three most |
| Comorbidities | common diagnoses ^{g} |
| Medical Comorbidities | Number of non-HCV diagnoses and three most common |
| | diagnoses ^{g} |

^{a} VA = US Department of Veterans Affairs, HCV = Hepatitis C Virus, PTSD = Posttraumatic Stress Disorder, PE = Prolonged Exposure, CPT = Cognitive Processing Therapy
^{b} Variable was assessed with a look-back period to Oct. 1, 1999.
^{c} FIB-4 grades level of hepatic fibrosis based on age, platelet counts, and aminotransferase levels (Sterling et al., 2006; Vallet-Pichard et al., 2007).
^{d} Including fluoxetine, paroxetine, sertraline, and venlafaxine (Bernardy N, 2017).
^{e} Measured using a natural language processing algorithm that classifies psychotherapy note text (Maguen et al., 2018).
^{f} Cutoff of 8 based on optimal diagnostic efficiency (Rubinsky et al., 2013).
^{g} Using a previously published index of mental and physical comorbidities adapted for VA medical record data (Shiner, Peltzman, et al., 2021).

Statistical Analysis: An analytic strategy developed for the prior study of changes in PTSD symptoms among patients with PTSD and HCV (Shiner, Huybrechts, et al., 2022) was followed. Briefly, the first step in the analysis was to balance confounders across the DAA medication exposure groups. Covariates with a standardized mean difference (SMD) of greater than or equal to 0.1 were considered as confounders for the univariate association with clinically meaningful improvement and for the bivariate relationship between exposure to one DAA versus each of the other DAAs (Austin, 2009). After identification of confounders, propensity scores representing the probability that a particular trial would be of each DAA combination were calculated (Stuart, 2010). Propensity scores were estimated with multinomial logistic regression using generalized booster effects (McCaffrey et al., 2013), in which the dependent variable was an indicator for each of the three medications and variables meeting the definition of confounders (i.e., variables associated with the exposure and the outcome with an SMD ≥0.1) were the independent variables (McCaffrey et al., 2013: Stuart, 2010). Weights were estimated based on the population average treatment effect model.

The second step in the analysis was to compare continuous and categorical AUD outcomes among the three DAA combinations with weighted regression analyses, using DAA combination received as the independent variable. These weighted medication groups were defined by the inverse of the propensity scores and adjusted for covariates that remained unbalanced at the SMD ≥0.1 level after propensity score weighting. For the categorical outcome of clinically meaningful improvement, weighted logistic regression analysis was used, whereby the coefficient of the variable tests the hypothesis that each of the three DAA combinations results in the same percentage of patients achieving clinically meaningful improvement. For the continuous outcome of change in AUDIT-C score, weighted linear regression analysis was used, whereby the coefficient of the variable tests the hypothesis that each of the three DAA combinations, coded as a multilevel categorical variable, has the same mean change from baseline to follow-up. Data management was performed in SAS version 9.4 (SAS Institute) and statistical modeling in R version 4.0.2 (R core team).

b. Results

A total of 1,211 patients met the inclusion criteria, including 174 who received GLE/PIB, 808 who received LDV/SOF, and 229 who received SOF/VEL (Table 14). There were many differences between patients across groups (Table 15), and twelve covariates met the definition of confounders based on having an association with both the exposure and the outcome at the SMD ≥0.1 level (Table 16). Patients receiving GLE/PIB had lower frequency of advanced liver fibrosis, higher frequency of military sexual trauma, and higher frequency of non-AUD SUDs. Patients receiving LDV/SOF had fewer days from first available AUD diagnosis to DAA initiation, more weeks of evidence-based antidepressants recommended by the VA for PTSD (EBAs for PTSD) concurrent with their DAA, fewer emergency department visits for psychiatric indications and psychiatric hospitalizations in the year preceding DAA initiation, as well as higher frequency of diabetes diagnoses in the two years prior to DAA initiation. Patients receiving SOF/VEL had fewer sessions of evidence-based psychotherapy protocols recommended by the VA for PTSD (EBP for PTSD) concurrent with their DAA. Patients in all three groups differed on the timing of their baseline and follow-up AUDIT-C measurements relative to their DAA trial and on whether they received FDA-approved medications for AUD. After propensity score weighting, seven confounders remained unbalanced and were retained as covariates in all weighted outcome models (Table 17). These included lower frequency of advanced fibrosis for the GLE/PIB group, more days from the baseline AUDIT-C score to DAA initiation in the LDV/SOF group, more concurrent sessions of EBP for PTSD in the LDV/SOF group, fewer weeks of concurrent EBA for PTSD in the GLE/PIB group, continued difference across groups in receipt of FDA-approved medications for AUD, higher frequency of non-AUD SUDs in the GLE/PIB group, and higher frequency of diabetes in the LDV/SOF group.

### Table 14.

| Question | | Answer |
|---|---|---|
| A | How many Department of Veterans Affairs (VA) patients | 2,098,389 |
| | had any diagnosis of PTSD between FY2000 and | |
| | FY2019? | |
| B | How many patients from (A) also had a diagnosis of | 147,174 |
| | Hepatitis C Virus (HCV)? | |
| C | How many patients from (B) also had a diagnosis of | 103,588 |
| | AUD? | |
| D | How many patients from (C) received at least one | 43,070 |
| | prescription of a DAA for HCV? | |
| E | How many patients from (D) received at least one | 27,606 |
| | prescription for GLE/PIB, LDV/SOF, or SOF/VEL? | |
| F | How many prescriptions from (E) followed a 6-month | 28,556 |
| | washout of that medication? | |
| G | How many prescriptions from (F) were continued for at | 21,870 |
| | least 56 days? | |
| H | How many starts from (G) had an Alcohol Use Disorder | 19,982 |
| | Identification Test (AUDIT-C) within 365 days prior to | |
| | 7 days after DAA start. | |
| I | How many starts from (H) had an AUDIT-C within 365 | 16,121 |
| | days after the 35-day point? | |
| J | How many person trials from (I) had their first AUD | 14,619 |
| | diagnosis prior to trial start? | |
| K | How many persons had a baseline AUDIT-C score of 8 | 1,211 |
| | or greater? | |

Detailed Information from (J): Person Trials from All Potential Patients

| GLE/PIB | LDV/SOF | SOF/VEL |
|---|---|---|
| 174 | 808 | 229 |

^{a} GLE/PIB = Glecaprevir/Pibrentasvir, LDV/SOF = Ledipasvir/Sofosbuvir, SOF/VEL = Sofosbuvir/Velpatasvir

### Table 15.

TABLE 15
Baseline Patient and Clinical Characteristics ^{a}

| | GLE/PIB | | LDV/SOF | | SOF/VEL | |
|---|---|---|---|---|---|---|
| | (N = 174) | | (N = 808) | | (N = 229) | |
| Drug Combination | n | % | n | % | n | % |

Hepatitis Disease Status

| | | | |
|---|---|---|---|
| Days Since HCV Diagnosis (M ± SD) | 3035.38 ± 2436.47 | 3330.59 ± 2044.97 | 3229.26 ± 2278.13 |

Fibrosis (FIB-4 Score)

| | | | | | | |
|---|---|---|---|---|---|---|
| Mild (<1.45) | 64 | 36.8 | 205 | 25.4 | 63 | 27.5 |
| Moderate (≥1.45 and ≤3.25) | 61 | 35.1 | 304 | 37.6 | 83 | 36.2 |
| Advanced (>3.25) | 26 | 14.9 | 181 | 22.4 | 55 | 24.0 |
| HCV cure | 157 | 90.2 | 775 | 95.9 | 208 | 90.8 |

Alcohol Use Disorder Status

| | | | |
|---|---|---|---|
| Days since Alcohol Use Disorder | 4074.65 ± 2215.98 | 3677.85 ± 2020.12 | 3987.87 ± 2269.33 |
| Diagnosis | | | |

Prior Evidence-Based PTSD Treatment

| | | | |
|---|---|---|---|
| Number of Prior EBA Trials (M ± SD) | 0.55 ± 1.03 | 0.36 ± 0.65 | 0.47 ± 0.91 |
| Number of Prior EBP Trials (M ± SD) | 0.10 ± 0.30 | 0.07 ± 0.27 | 0.08 ± 0.31 |

Alcohol Use Disorder Timing, Version, and Severity

| | | | |
|---|---|---|---|
| Days from Earliest Baseline AUDIT- | 92.07 ± 89.88 | 139.29 ± 97.37 | 112.20 ± 92.63 |
| C to start of DAA treatment (M ± SD) | | | |
| Days from Baseline AUDIT-C to | 277.55 ± 138.48 | 329.28 ± 116.32 | 297.03 ± 137.84 |
| Follow-Up AUDIT-C (M ± SD) | | | |
| Days from DAA End to Follow-Up | 124.85 ± 102.06 | 118.96 ± 91.99 | 113.05 ± 94.09 |
| AUDIT-C (M ± SD) | | | |
| Baseline AUDIT-C (M ± SD) | 10.42 ± 1.44 | 10.25 ± 1.50 | 10.21 ± 1.48 |

Concurrent Treatment

| | | | | | | |
|---|---|---|---|---|---|---|
| Sessions of EBP for PTSD (M ± SD) | 0.74 ± 2.76 | | 0.63 ± 2.87 | | 0.38 ± 2.13 | |
| Weeks of EBA for PTSD (M ± SD) | 4.90 ± 9.77 | | 6.86 ± 12.30 | | 5.62 ± 10.14 | |
| Any Non-EBA Antidepressant | 118 | 67.8 | 516 | 63.9 | 139 | 60.7 |
| Any Anticonvulsant | 41 | 23.6 | 148 | 18.3 | 35 | 15.3 |
| Any Sedative/Hypnotics | 17 | 9.8 | 141 | 17.5 | 31 | 13.5 |
| Any Opioid | 55 | 31.6 | 306 | 37.9 | 58 | 25.3 |
| Any Atypical Antipsychotic | 53 | 30.5 | 241 | 29.8 | 62 | 27.1 |
| Any Prazosin | 36 | 20.7 | 158 | 19.6 | 40 | 17.5 |
| Any FDA Approved Medications for AUD | 47 | 27.0 | 131 | 16.2 | 51 | 22.3 |
| Any Opioid Agonist Therapy | 24 | 13.8 | 65 | 8.0 | 21 | 9.2 |

Patient Characteristics at Baseline

| | | | | | | |
|---|---|---|---|---|---|---|
| Age (M ± SD) | 55.31 ± 11.40 | | 58.10 ± 9.21 | | 56.87 ± 10.93 | |
| Women | 8 | 4.6 | 26 | 3.2 | 7 | 3.1 |
| Married | 29 | 16.7 | 152 | 18.8 | 43 | 18.8 |
| Rural | 40 | 23.0 | 172 | 21.3 | 63 | 27.5 |
| White Non-Hispanic | 112 | 64.4 | 408 | 50.5 | 161 | 70.3 |
| Black Non-Hispanic | 47 | 27.0 | 331 | 41.0 | 44 | 19.2 |
| Hispanic | 7 | 4.0 | 38 | 4.7 | 16 | 7.0 |
| Other Racial or Ethnic Group | 6 | 3.4 | 13 | 1.6 | 5 | 2.2 |
| Combat Exposure | 41 | 23.6 | 162 | 20.0 | 57 | 24.9 |
| Sexual Trauma while in Military | 36 | 20.7 | 133 | 16.5 | 41 | 17.9 |
| VA Disability Level 70% or Greater | 49 | 28.2 | 229 | 28.3 | 65 | 28.4 |

Service Use Characteristics in the 1 Year Preceding Baseline

| | | | |
|---|---|---|---|
| Number of PTSD Outpatient Clinical | 2.54 ± 9.43 | 3.85 ± 16.23 | 3.10 ± 14.41 |
| Team Visits (M ± SD) | | | |
| Number of Outpatient Mental Health | 104.53 ± 98.11 | 96.58 ± 102.96 | 85.89 ± 93.07 |
| Visits (M ± SD) | | | |
| Number of Outpatient Substance | 30.49 ± 37.29 | 40.04 ± 58.18 | 29.21 ± 40.81 |
| Abuse Visits (M ± SD) | | | |
| Number of Outpatient Primary Care | 10.20 ± 9.14 | 11.23 ± 8.91 | 9.858 ± 38 |
| Visits (M ± SD) | | | |
| Number of Outpatient Specialty | 3.99 ± 3.15 | 4.55 ± 3.69 | 4.51 ± 3.57 |
| Medical Visits (M ± SD) | | | |
| Number of ED Visits for Psychiatric | 2.26 ± 3.53 | 1.65 ± 3.17 | 2.06 ± 3.45 |
| Indication (M ± SD) | | | |
| Days of Acute Inpatient Mental | 61.93 ± 118.33 | 50.51 ± 100.20 | 61.94 ± 106.82 |
| Health (M ± SD) | | | |
| Days of Residential PTSD Treatment | 9.67 ± 33.56 | 8.84 ± 33.29 | 9.27 ± 34.00 |
| (M ± SD) | | | |
| Days Residential Substance Abuse | 53.50 ± 89.63 | 49.13 ± 88.20 | 49.07 ± 86.53 |
| Treatment (M ± SD) | | | |

Comorbidities in the 2 Years Preceding Baseline

| | | | | | | |
|---|---|---|---|---|---|---|
| Number of Non-Alcohol Use | 3.30 ± 2.06 | | 2.92 ± 1.83 | | 3.02 ± 1.81 | |
| Disorder MH Comorbidities (M ± SD) | | | | | | |
| Trauma | 117 | 67.2 | 544 | 67.3 | 156 | 68.1 |
| Depression | 125 | 71.8 | 581 | 71.9 | 163 | 71.2 |
| Substance use disorder | 143 | 82.2 | 620 | 76.7 | 174 | 76.0 |
| Number of Non-HCV PH | 2.73 ± 2.64 | | 2.74 ± 2.32 | | 2.75 ± 2.50 | |
| Comorbidities (M ± SD) | | | | | | |
| Uncomplicated Hypertension | 93 | 53.4 | 521 | 64.5 | 129 | 56.3 |
| Diabetes | 19 | 10.9 | 142 | 17.6 | 28 | 12.2 |
| Liver disease | 50 | 28.7 | 271 | 33.5 | 90 | 39.3 |

^{a} DAA = Direct-Acting Antiviral; PTSD = posttraumatic stress disorder, FY = Fiscal Year; VA = Department of Veterans Affairs; HCV = Hepatitis C Virus; EBA = Evidence-Based Antidepressant; EBP = Evidence-Based Psychotherapy PCL = PTSD Checklist; MH = Mental Health; PH = Physical Health; SL = suppressed low: cell is suppressed due to less than 3 patients; SH = suppressed high: cell is suppressed due to all but fewer than 3 patients

### Table 16.

TABLE 16
Assessment of Potential Confounding, as Measured
by Absolute Standardized Mean Difference ^{a b}

| | Association | | | |
|---|---|---|---|---|
| | | Exposure | | |
| | | GLE/PIB | GLE/PIB | SOF/VEL |
| | Outcome | versus | versus | versus |
| Variable | No misuse ^{c} | LDV/SOF | SOF/VEL | LDV/SOF |

Hepatitis Disease Status

| | | | | |
|---|---|---|---|---|
| Thousands of Days Since | 0.05 | 0.13 | 0.08 | 0.05 |
| HCV Diagnosis | | | | |

Fibrosis (FIB-4 Score)

| | | | | |
|---|---|---|---|---|
| Mild (<1.45) | 0.05 | 0.25 | 0.20 | 0.05 |
| Moderate | 0.06 | 0.05 | 0.03 | 0.03 |
| (≥1.45 and ≤3.25) | | | | |
| Advanced (>3.25) | 0.11 | 0.19 | 0.23 | 0.04 |

Alcohol Use Disorder Status

| | | | | |
|---|---|---|---|---|
| Thousands of Days since | 0.10 | 0.19 | 0.04 | 0.14 |
| Alcohol Use Disorder | | | | |
| Diagnosis | | | | |

Prior Evidence-Based PTSD Treatment

| | | | | |
|---|---|---|---|---|
| Number of Prior EBA Trials | 0.02 | 0.22 | 0.08 | 0.14 |
| Number of Prior EBP Trials | 0.06 | 0.11 | 0.06 | 0.05 |

AUDIT-C Timing, Version, and Severity

| | | | | |
|---|---|---|---|---|
| Days from DAA Start to | 0.26 | 0.50 | 0.22 | 0.29 |
| Earliest Baseline AUDIT-C | | | | |
| Days from Baseline AUDIT- | 0.24 | 0.41 | 0.14 | 0.25 |
| C to Follow-Up AUDIT-C | | | | |
| Days from DAA End to | 0.02 | 0.06 | 0.12 | 0.06 |
| Follow-Up AUDIT-C | | | | |
| Baseline AUDIT-C | 0.05 | 0.12 | 0.14 | 0.03 |

Concurrent Treatment

| | | | | |
|---|---|---|---|---|
| Sessions of EBP for PTSD | 0.14 | 0.04 | 0.15 | 0.10 |
| Weeks of EBA for PTSD | 0.22 | 0.18 | 0.07 | 0.11 |
| Any Non-EBA | 0.06 | 0.08 | 0.15 | 0.07 |
| Antidepressant | | | | |
| Any Anticonvulsant | 0.05 | 0.13 | 0.21 | 0.08 |
| Any Sedative/Hypnotics | 0.04 | 0.23 | 0.12 | 0.11 |
| Any Opioid | 0.08 | 0.13 | 0.14 | 0.27 |
| Any Atypical Antipsychotic | 0.06 | 0.01 | 0.08 | 0.06 |
| Any Prazosin | 0.08 | 0.03 | 0.08 | 0.05 |
| Any FDA Approved | 0.10 | 0.27 | 0.11 | 0.15 |
| Medications for AUD | | | | |
| Any Opioid Agonist Therapy | 0.06 | 0.19 | 0.15 | 0.04 |

Patient Characteristics at Baseline

| | | | | |
|---|---|---|---|---|
| Age | 0.08 | 0.27 | 0.14 | 0.12 |
| Women | 0.02 | 0.07 | 0.08 | 0.01 |
| Married | 0.08 | 0.06 | 0.06 | 0 |
| Rural | 0.06 | 0.04 | 0.10 | 0.15 |
| White Non-Hispanic | 0.08 | 0.28 | 0.13 | 0.41 |
| Black Non-Hispanic | 0.07 | 0.30 | 0.19 | 0.49 |
| Hispanic | 0.03 | 0.03 | 0.13 | 0.10 |
| Other Racial or Ethnic Group | 0.01 | 0.12 | 0.08 | 0.04 |
| Combat Exposure | 0.07 | 0.09 | 0.03 | 0.12 |
| Sexual Trauma while in | 0.12 | 0.11 | 0.07 | 0.04 |
| Military | | | | |
| VA Disability Level 70% or | 0 | 0 | 0.01 | 0 |
| Greater | | | | |

Service Use Characteristics in the 1 Year Preceding Baseline

| | | | | |
|---|---|---|---|---|
| Number of PTSD Outpatient | 0.02 | 0.10 | 0.05 | 0.05 |
| Clinical Team Visits | | | | |
| Number of Outpatient Mental | 0.02 | 0.08 | 0.20 | 0.11 |
| Health Visits | | | | |
| Number of Outpatient | 0.04 | 0.20 | 0.03 | 0.22 |
| Substance Abuse Visits | | | | |
| Number of Outpatient | 0.08 | 0.11 | 0.04 | 0.16 |
| Primary Care Visits | | | | |
| Number of Outpatient | 0.02 | 0.17 | 0.16 | 0.01 |
| Specialty Medical Visits | | | | |
| Number of ED Visits for | 0.27 | 0.18 | 0.06 | 0.12 |
| Psychiatric Indication | | | | |
| Days of Acute Inpatient | 0.24 | 0.10 | <0.001 | 0.11 |
| Mental Health | | | | |
| Days of Residential PTSD | 0.03 | 0.03 | 0.01 | 0.01 |
| Treatment | | | | |
| Days Residential Substance | 0.19 | 0.05 | 0.05 | 0 |
| Abuse Treatment | | | | |

Comorbidities in the 2 Years Preceding Baseline

| | | | | |
|---|---|---|---|---|
| Number of Non-Alcohol Use | 0.01 | 0.19 | 0.15 | 0.05 |
| Disorder MH Comorbidities | | | | |
| (M ± SD) | | | | |
| Trauma | 0.04 | 0 | 0.02 | 0.02 |
| Depression | 0.03 | 0 | 0.02 | 0.02 |
| Substance use disorder | 0.13 | 0.14 | 0.15 | 0.02 |
| Number of Non-HCV PH | 0.05 | 0.01 | 0.01 | 0 |
| Comorbidities (M ± SD) | | | | |
| Uncomplicated | 0.02 | 0.23 | 0.06 | 0.17 |
| Hypertension | | | | |
| Diabetes | 0.15 | 0.19 | 0.04 | 0.15 |
| Liver disease | 0.04 | 0.10 | 0.22 | 0.12 |

^{a} DAA = Direct-Acting Antiviral; PTSD = posttraumatic stress disorder, FY = Fiscal Year; VA = Department of Veterans Affairs; HCV = Hepatitis C Virus; EBA = Evidence-Based Antidepressant; EBP = Evidence-Based Psychotherapy; PHQ-9 = Patient Health Questionnaire-9; MH = Mental Health; PH = Physical Health
^{b} Covariates associated with both the outcome and exposure (SMD ≥ 0.10) are indicated by the gray rows.
^{c} Decrease to 3 points or less on AUDIT-C for men; decrease to 2 points or less on AUDIT-C for women.

### Table 17.

TABLE 17
Post-Weighting Balance of Confounders ^{a}

| | GLE/PIB | LDV/SOF | SOF/VEL |
|---|---|---|---|
| Drug combination | (N = 174) | (N = 808) | (N = 229) |
| Advanced Fibrosis (>3.25) ^{b} | 15.4% | 22.5% | 23.1% |
| Thousands of Days since Alcohol Use | 3751 ± 2124 | 3714 ± 2050 | 3789 ± 2204 |
| Disorder Diagnosis (M ± SD) | | | |
| Days from DAA Start to Earliest Baseline | 121 ± 97 | 131 ± 97 | 119 ± 92 |
| AUDIT-C (M ± SD) ^{b} | | | |
| Days from Baseline AUDIT-C to Follow-Up | 318 ± 138 | 320 ± 122 | 312 ± 132 |
| AUDIT-C (M ± SD) | | | |
| Sessions of EBP for PTSD (M ± SD) ^{b} | 0.4 ± 2 | 0.6 ± 3 | 0.4 ± 2 |
| Weeks of EBA for PTSD (M ± SD) ^{b} | 5 ± 9 | 6 ± 12 | 6 ± 11 |
| Any FDA Approved Medications for AUD ^{b} | 19.9% | 17.3% | 22.2% |
| Sexual Trauma while in Military | 17.2% | 16.7% | 16.6% |
| Number of ED Visits for Psychiatric | 2 ± 3 | 2 ± 3 | 2 ± 3 |
| Indication (M ± SD) | | | |
| Days of Acute Inpatient Mental Health | 47 ± 100 | 52 ± 102 | 54 ± 99 |
| (M ± SD) | | | |
| Substance use disorder ^{b} | 81.9% | 77.6% | 75.2% |
| Diabetes ^{b} | 14.6% | 16.3% | 11.4% |

^{a} HCV = Hepatitis C Virus; PH = Physical health
^{b} Variable was not balanced (SMD > 0.10) after weighting and was retained as a covariate in outcomes models.

Consistent with the patient selection strategy, crude and adjusted AUDIT-C scores were approximately 10 at baseline (Table 18), reflecting heavy alcohol consumption. Crude frequency of clinically meaningful improvement ranged from 44.3% in the GLE/PIB group to 59.9% in the LDV/SOF group, although adjustment for differences between groups substantially lowered these proportions to 30.5% and 45.5%, respectively. This reflected a mean adjusted change in AUDIT-C scores ranging from −4.24 (SD=6.90) in the GLE/PIB group to −5.43 (SD=10.16) in the LDV/SOF group. In direct comparisons (Table 19), GLE/PIB performed worse than LDV/SOF, including both lower odds of clinically meaningful improvement (OR=0.59; 95% CI [0.40, 0.87]) and a smaller mean change in AUDIT-C score (MD=−1.20: 95% CI [−2.03,−0.36]). Differences in categorical and continuous outcomes in other bivariate comparisons were smaller in magnitude.

### Table 18.

TABLE 18
Distribution of Baseline AUDIT-C and
Outcomes Before and After Weighting ^{a}

| | | |
|---|---|---|
| GLE/PIB | LDV/SOF | SOF/VEL |
| (N = 174) | (N = 808) | (N = 229) |
| M ± SD | M ± SD | M ± SD |

Baseline AUDIT-C

| | | | |
|---|---|---|---|
| Unweighted | 10.42 ± 1.44 | 10.25 ± 1.50 | 10.21 ± 1.48 |
| IPTW | 10.11 ± 2.46 | 10.09 ± 7.05 | 9.98 ± 3.92 |

Change in AUDIT-C

| | | | |
|---|---|---|---|
| Unweighted | −5.28 ± 4.68 | −6.66 ± 4.26 | −5.59 ± 4.75 |
| IPTW | −4.24 ± 6.90 | −5.43 ± 19.16 | −4.66 ± 11.01 |

Clinically Meaningful Improvement (decrease to 3 points or
less on AUDIT-C for men; decrease to 2 points or less on
AUDIT-C for women)

| | n | % | n | % | n | % |
|---|---|---|---|---|---|---|
| Unweighted | 77 | 44.3 | 484 | 59.9 | 118 | 51.5 |
| IPTW | 77 | 30.5 | 484 | 45.5 | 118 | 40.5 |

^{a} AUDIT-C = Alcohol Use Disorders Identification Test, M = Mean, SD = Standard Deviation, IPTW = Inverse Probability of Treatment Weighting

### Table 19.

TABLE 19
Comparison Baseline AUDIT-C and
Outcomes Across DAA Treatments ^{a}

| GLE/PIB versus | | GLE/PIB versus | | SOF/VEL versus | |
|---|---|---|---|---|---|
| LDV/SOF | | SOF/VEL | | LDV/SOF | |
| MD | 95% CI | MD | 95% CI | MD | 95% CI |

Baseline AUDIT-C

| | | | | | | |
|---|---|---|---|---|---|---|
| Unweighted | −0.17 | −0.41, | −0.21 | −0.50, | 0.04 | −0.18, |
| | | 0.07 | | 0.08 | | 0.26 |
| IPTW | −0.02 | −0.34, | −0.13 | −0.48, | 0.11 | −0.12, |
| | | 0.30 | | 0.22 | | 0.33 |

Change in AUDIT-C

| | | | | | | |
|---|---|---|---|---|---|---|
| Unweighted | −1.38 | −2.14, | −0.31 | −1.24, | −1.07 | −1.76, |
| | | −0.62 | | 0.62 | | −0.39 |
| IPTW | −1.20 | −2.03, | −0.42 | −1.41, | −0.66 | −1.37, |
| | | −0.36 | | 0.57 | | 0.05 |

Clinically Meaningful Improvement (decrease to 3 points
or less on AUDIT-C for men; decrease to 2 points or less
on AUDIT-C for women)

| | OR | 95% CI | OR | 95% CI | OR | 95% CI |
|---|---|---|---|---|---|---|
| Unweighted | 0.74 | 0.62, 0.88 | 0.86 | 0.70, 1.06 | 0.86 | 0.75, 0.99 |
| IPTW | 0.59 | 0.40, 0.87 | 0.66 | 0.42, 1.04 | 0.89 | 0.65, 1.21 |

^{a} DAA = Direct Acting Antiviral, MD = Mean Difference, OR = Odds Ratio, CI = Confidence Interval, AUDIT-C = Alcohol Use Disorders Identification Test, M = Mean, SD = Standard Deviation, IPTW = Inverse Probability of Treatment Weighting

c. Discussion

In a cohort of VA patients with a diagnosis of HCV, AUD, and co-occurring PTSD, a substantial reduction of alcohol use was observed in all three DAA treatment groups, with mean improvements in AUDIT-C scores between 4.2 and 5.4 points. Similarly, in considering alcohol use as a categorical outcome, each DAA resulted in large proportions of patients with final AUDIT-C scores below the threshold for misuse, again ranging from 30% to 45% depending on the specific medication. After adjustment for differences between treatment groups, LDV/SOF appears to have the strongest association with clinically meaningful improvement in alcohol consumption compared to GLE/PIB and SOF/VEL. This result may be explained by LDV/SOF being the only medication in the analysis that was available prior to the 2016 HCV treatment directives around alcohol abstinence and liver health status. Furthermore, the smaller decrease in alcohol consumption observed in the GLE/PIB and SOF/VEL groups may be explained by the timing of their release falling after the directive. Notably, baseline AUDIT-C scores were obtained up to 365 days prior to DAA initiation, and LDV/SOF had the most days (139.29±97.37 days) from baseline AUDIT-C to DAA start. This may have allowed a longer period to achieve abstinence prior to HCV treatment.

In contrast to findings in previous work demonstrating greater PTSD improvement on GLE/PIB compared with the other DAAs (Shiner, Huybrechts, et al., 2022), GLE/PIB was associated with the smallest improvement in drinking outcomes in the current analysis. This is notable, as an observation of greater decreases in alcohol consumption for patients prescribed GLE/PIB compared to the other agents may have indicated AUD as a potential mediator of the GLE/PIB and PTSD association. While sample size issues preclude conducting formal mediation tests of these associations, this finding provides some evidence that changes in alcohol consumption are unlikely to be a mechanism of the previously documented association between GLE/PIB and improvements in PTSD symptoms (Shiner, Huybrechts, et al., 2022).

The evidence regarding effectiveness for AUD treatment in HCV populations have not previously been impressive. For example, a study comparing provider delivered Screening, Brief Intervention and Referral to Treatment (SBIRT) versus intensive SBIRT and integrated alcohol counseling, in a liver clinic treating HCV, found no enhancement over standard of care, and only 19-21% of participants achieved abstinence from alcohol (Proeschold-Bell et al., 2020). In a systematic review of psychosocial interventions for alcohol abstinence in patients with chronic liver disease, no psychosocial intervention demonstrated success (Khan et al., 2016). Thus, available evidence around expected alcohol consumption reduction in liver clinic treatment settings would suggest that the observed improvement both overall and within DAA treatment group is compelling.

Furthermore, the observed drinking reductions in the cohort may be higher than that observed with standard psychopharmacology for AUD (Kranzler & Soyka, 2018). Currently, there are three FDA-approved agents for the treatment of AUD including disulfiram and the two frontline treatments, acamprosate and oral or long-acting injectable formulations of naltrexone (Kranzler & Soyka, 2018). Additionally, several off-label medications including gabapentin and topiramate are currently utilized as second-line AUD treatments (Swift & Aston, 2015). These disparate medications, unified by their indication rather than mechanism of action, have fairly limited efficacy with effect sizes in the small to medium range compared to placebo and the literature calls for investigation into more efficacious medications (Kranzler & Soyka, 2018).

Mechanistically, it is unclear why GLE/PIB would be associated with the greatest PTSD improvement and the smallest drinking reduction. Notably, GLE/PIB is the only agent in this analysis to contain a serine protease inhibitor of HCV non-structural viral protein NS3/4A (Lamb, 2017). Serine protease inhibitors are known to act in the interferon signaling pathway which plays a critical role in the innate immune response (Casella et al., 2020). Overexpression of interferon signaling in the innate immune response has been implicated in PTSD pathophysiology (Breen et al., 2015). If GLE/PIB were to inhibit the interferon signaling pathway it is plausible that PTSD symptoms could improve.

All three DAA agents in the analysis contain NS5A inhibitors. NS5A is an HCV non-structural viral protein that is known to exploit protein kinase R (PKR) activity in immune cells during the viral replication process (He et al., 2001: Suzuki et al., 2019). It has been demonstrated that PKR regulates NLRP3 activation (Lan et al., 2020; Lu et al., 2012). It is plausible that NS5A inhibitors may also inhibit their cellular counterpart, PKR, whose signaling in immune cells has a demonstrated role in neuroinflammation (Gal-Ben-Ari et al., 2019; Kapil et al., 2014), possibly due to monocyte trafficking to the brain (Wohleb et al., 2014). More specifically, PKR inhibitors have been identified as potential inhibitors of nuclear factor kappa B (NF-KB) protein complex (Gupta et al., 2010; Zhang et al., 2013), a transcription factor that has demonstrated the ability to induce genetic expression involved in addiction pathways including opioid receptors (Nennig & Schank, 2017). With the identification of NLRP3 as a common component in HCV, PTSD, and AUD, the potential for a DAA acting in this pathway with off-target addiction effects emerges, which may have been masked by expected alcohol consumption reduction in the context of HCV treatment. Replication of results in a larger, less restrictive sample of patients receiving the most recent DAAs for HCV would be beneficial to future research in this area. Furthermore, prospectively testing DAAs in a non-HCV sample of patients with AUD would be an important next step in identifying a potential AUD medication with an alternative mechanism of action.

d. Conclusion

Patients with co-occurring AUD and PTSD who are receiving DAA treatment for HCV experience a substantial reduction in alcohol use. Further exploration of the role of DAAs in AUD treatment is warranted.

### H. PROPHETIC EXAMPLES

### 1. Pilot Study

An open label pilot trial of GLE/PIB for the treatment of PTSD will be conducted in patients eligible to receive care at the White River Junction Veterans Affairs Medical Center (VAMC). This study will be done as preparatory work for larger, external funded studies of GLE/PIB for PTSD. The study will fit within the FDA framework for drug approval as a Phase 2 study, given that there are extensive data already available regarding the safety and tolerability of GLE/PIB. GLE/PIB is already approved as a treatment for chronic hepatitis C virus (HCV) infections by the FDA. The goal of this Phase 2 study would be to gather preliminary data regarding the efficacy of GLE/PIB as a treatment for PTSD.

Composition of study population and rationale. This proposed trial will enroll approximately 10 patients between 19 and 70 years old with PTSD. Potential subjects will be eligible for care at the White River Junction VAMC. This site has a substantial population of patients with PTSD who would be potential participants in the clinical trial. In addition, approximately 8 men and 2 women are expected to be enrolled based on patient demographics at WRJ VAMC.

Drugs to be used. Glecaprevir 100 mg/Pibrentasvir 40 mg. 3 oral tablets once daily for 8 weeks. Drugs will be purchased with study funds from the national medical formulary available from VA's Pharmacy Benefits Management (PBM) program. All medications will be dispensed by the White River Junction VAMC pharmacy.

Eligibility and Primary Outcome Measure. Clinician Administered PTSD Scale (CAPS) (Weathers et al. (2018) Psychol Assess. 30 (3): 383-395) is a clinician-administered scale of PTSD symptoms, which queries the frequency and intensity of symptoms of PTSD. It is considered the gold standard for diagnosis and symptoms assessment in PTSD clinical studies. The Life Events Checklist (LEC) will be administered as a precursor to the CAPS as a brief (a few minutes) tool for identifying an index trauma. The LEC-5 assesses exposure to 16 events known to potentially result in PTSD or distress and includes one additional item assessing any other extraordinarily stressful event not captured in the first 16 items.⁸¹ If no traumatic event is indicated, the patient would screen fail and therefore would not proceed to the CAPS. Thus, the LEC helps prevent an unnecessary CAPS assessment. The primary outcome will consider CAPS score as a continuous value assessed just after completing the eight-week trial of GLE/PIB. The use of CAPS will be based on DSM-V criteria and will use the version that queries symptoms over the past week. The CAPS will be done at baseline to establish eligibility for study participation, after 4 weeks of treatment (midpoint), after completion of 8 weeks of treatment (treatment completion). 3 months after treatment completion, and 6 months after treatment completion. CAPS interviews will be audio-recorded directly into the research server study folder using Audacity software on the assessor's VA laptop and a Fifine Podcast Microphone K670.670B. A random sample of 5% of CAPS recordings will be selected for inter-rater reliability.

To evaluate whether GLE/PIB treatment will be associated with improvement in PTSD symptoms, linear mixed models will be used to test the treatment-by-time interaction on the CAPS. The primary outcome will be the post-treatment CAPS. The extent by which an improvement is realized will be assessed by comparing baseline and post treatment CAPS scores.

Exploratory blood bio-marker analysis. For the eligibility assessment, up to 40 milliliters will be drawn for the purposes of checking liver function, hepatitis B and C history, and exploratory analyses. At the post-treatment assessment, up to 40 milliliters of blood will be drawn for exploratory analyses as well as to check liver function, consistent with clinical prescribing guidelines for GLE/PIB. Exploratory analyses for the purpose of investigating a possible biological mechanism of GLE/PIB may include genetic expression of interferon production as well as the measurement of non-coding RNAs and pro-inflammatory cytokines, consistent with current PTSD bio-marker research (Breen et al. (2015) Molecular Psychiatry 20 (12): 1538-1545: Daskalakis et al. (2018) Biol Psychiatry. 83 (10): 849-865: Passos et al. (2015) Lancet Psychiatry. 2015: 2 (11): 1002-1012). Liver function and history of hepatitis will be tested and handled in a manner consistent with clinical practice. Vials for exploratory analyses will be labeled with the participant's assigned study ID and taken to Building 44 for processing. Data from these analyses will be entered directly into the secure research study folder under the participant's assigned study ID. The master list on the study folder will link the patient's study ID to their name. Samples will be destroyed immediately after analysis.

Additional Outcome Measures. The World Health Organization Disability Assessment Schedule 2.0 (WHODAS) (Buist-Bouwman et al. (2008) Int J Methods Psychiatr Res. 17 (4): 185-197) is a 36-item self-report instrument that assesses disability and function across six domains: communicating, getting around, self-care, getting along with people, life activities, and participation in society. The WHODAS has been used as an outcome of function and disability across many disorders and is commonly used in mental health treatment trials. The WHODAS will be done at the same interval as the CAPS.

To evaluate whether GLE/PIB treatment will be associated with improvement in functioning, linear mixed models will be used to test the treatment-by-time interaction on the WHODAS, and appropriate comparisons of model estimates (contrasts) will be performed to assess changes over time. The primary outcome will be the post-treatment WHODAS. The extent by which an improvement is realized will be assessed by comparing baseline and post treatment WHODAS scores.

The Systematic Assessment for Treatment Emergent Side Effects (SAFTEE) (Guy et al. (1987) Psychopharmacol Bull. 23 (1): 93-96: Guy et al. (1986) Psychopharmacol Bull. 22 (2): 397-401) is a 132-item self-report query regarding possible treatment emergent side effects. Initially developed by the National Institutes of Health in the 1980's it is widely used in psychopharmacology studies in all clinical disorders. It will be used to collect information about the rates and severity of side effects for each treatment group including sham. It will be assessed at treatment completion.

Compliance will be monitored by counts of returned medication. In addition, patients will be asked to report their degree of compliance to the medications at each visit. Specifically, participants will be requested bring their medication to each clinic visit. At each visit the study assessor will examine the medications and assess number of missing pills in comparison to the number that should have been taken. This number will be recorded in the study data capture and considered in future analysis. For situations where participants either forget medication bottle or receive a follow-up by virtual modality patients will be queried regarding these counts during the visit (or a follow-up phone call).

Interim safety checks will be performed at all phone and in-person visits. This will include questions about medication compliance described above, initiation of new medications or supplements to be checked against a list of possible interactions, side effects, adverse events, and suicidal ideation. This “Safety Check” questionnaire is included in the Appendix with other questionnaires.

To determine the occurrence of common side effects during treatment with GLE/PIB, the occurrence and proportion of patients experiencing each side effect will be described. The proportion of participants reporting each of the possible side effects listed in the SAFTEE scale will be reported.

### 2. Randomized Control Trial (RCT) of Mavyret® for PTSD

A double-blind, randomized placebo-controlled trial of GLE/PIB for the treatment of PTSD will be conducted in patients eligible to receive care at the White River Junction VAMC. The study will fit within the FDA framework for drug approval as a Phase 2 study; given that there are extensive data already available regarding the safety and tolerability of GLE/PIB as noted above. The goal of this Phase 2 study would be to gather preliminary data regarding the efficacy of GLE/PIB as a treatment for PTSD.

Composition of study population and rationale. This proposed trial will enroll approximately 92 patients between 19 and 70 years old with PTSD. Potential subjects will be eligible for care at the White River Junction VAMC. Tis site has a substantial population of patients with PTSD who would be potential participants in the clinical trial. VA data sources were queried to determine the population of patients eligible for participation. In addition, a selection of medical records was randomly sampled to determine what proportion of patients with PTSD would likely meet all inclusion and exclusion criteria. There are about 3,000 patients with a diagnosis of PTSD seen at White River Junction VAMC. A review of a sample of these medical records suggests that approximately 82% would meet study eligibility criteria. Thus, 3.7% of the likely eligible population will need to be recruited for the study.

Drugs to be used. Glecaprevir 100 mg/Pibrentasvir 40 mg, 3 oral tablets once daily for 8 weeks. As a sub-award site on this project, the VA Cooperative Studies Program Clinical Research Pharmacy Coordinating Center (VA CSP CRPCC) will provide active drugs and placebo directly to the White River Junction VAMC Pharmacy.

Eligibility and Primary Outcome Measure. As noted above, CAPS is a clinician-administered scale of PTSD symptoms, which queries the frequency and intensity of symptoms of PTSD, and is considered the gold standard for diagnosis and symptoms assessment in PTSD clinical studies. Here, the primary outcome will consider CAPS-5 score as a continuous value assessed just after completing the eight-week trial of GLE/PIB. The use of CAPS-5 will be based on DSM-V criteria and will use the version that queries symptoms over the past week. The CAPS-5 will be done at baseline to establish eligibility for study participation, after 4 weeks of treatment (midpoint), after completion of 8 weeks of treatment (treatment completion), 3 months after treatment completion, and 6 months after treatment completion. CAPS-5 interviews will be audio-recorded directly into the research server study folder using Audacity software on the assessor's VA laptop and a Fifine Podcast Microphone K670.670B. A random sample of 5% of CAPS-5 recordings will be selected for inter-rater reliability.

To evaluate whether GLE/PIB treatment will be associated with improvement in PTSD symptoms, linear mixed models will be used to test the treatment-by-time interaction on the CAPS-5. The primary outcome will be the post-treatment CAPS-5. The extent by which an improvement is realized will be assessed by comparing baseline and post treatment CAPS-5 scores.

Additional Outcome Measures. To evaluate whether GLE/PIB treatment will be associated with improvement in functioning, linear mixed models will be used to test the treatment-by-time interaction on the WHODAS (done at the same interval as the CAPS-5) (Buist-Bouwman et al. (2008) Int J Methods Psychiatr Res. 17 (4): 185-197), and appropriate comparisons of model estimates (contrasts) will be performed to assess changes over time. The primary outcome will be the post-treatment WHODAS. The extent by which an improvement is realized will be assessed by comparing baseline and post treatment WHODAS scores.

SAFTEE (Guy et al. (1987) Psychopharmacol Bull. 23 (1): 93-96: Guy et al. (1986) Psychopharmacol Bull. 22 (2): 397-401) will be used to collect information about the rates and severity of side effects for each treatment group including sham. It will be assessed at treatment completion.

Compliance will be monitored by counts of returned medication. In addition, patients will be asked to report their degree of compliance to the medications at each visit. Specifically, participants will be requested bring their medication to each clinic visit. At each visit the study assessor will examine the medications and assess number of missing pills in comparison to the number that should have been taken. This number will be recorded in the study data capture and considered in future analysis. For situations where participants either forget medication bottle or receive a follow-up by virtual modality patients will be queried regarding these counts during the visit (or a follow-up phone call).

Interim safety checks will be performed at all phone and in-person visits. This will include questions about medication compliance described above, initiation of new medications or supplements to be checked against a list of possible interactions, side effects, adverse events, and suicidal ideation. This “Safety Check” questionnaire is included in the Appendix with other questionnaires.

To determine the occurrence of common side effects during treatment with GLE/PIB, the occurrence and proportion of patients experiencing each side effect will be described. The proportion of participants reporting each of the possible side effects listed in the SAFTEE scale will be reported.

### I. REFERENCES

### 1. Treescan for PTSD Medications

Gradus J L, Antonsen S, Svensson E, Lash T L, Resick P A, Hansen J G (2015): Trauma, comorbidity, and mortality following diagnoses of severe stress and adjustment disorders: a nationwide cohort study. Am J Epidemiol. 182:451-458.
Hoge C W, Terhakopian A, Castro C A, Messer S C, Engel C C (2007): Association of posttraumatic stress disorder with somatic symptoms, health care visits, and absenteeism among Iraq war veterans. Am J Psychiatry. 164:150-153.
Pietrzak R H, Goldstein R B, Southwick S M, Grant B F (2011): Prevalence and Axis I comorbidity of full and partial posttraumatic stress disorder in the United States: results from Wave 2 of the National Epidemiologic Survey on Alcohol and Related Conditions. J Anxiety Disord. 25:456-465.
Shiner B, Leonard C E, Gui J, Cornelius S L, Schnurr P P, Hoyt J E, et al. (2020): Comparing Medications for DSM-5 PTSD in Routine V A Practice. J Clin Psychiatry. 81.
Shiner B, Westgate C L, Gui J, Maguen S, Young-Xu Y, Schnurr P P, et al. (2018): A Retrospective Comparative Effectiveness Study of Medications for Posttraumatic Stress Disorder in Routine Practice. J Clin Psychiatry. 79.
Krystal J H, Davis L L, Neylan T C, M A R, Schnurr P P, Stein M B, et al. (2017): It Is Time to Address the Crisis in the Pharmacotherapy of Posttraumatic Stress Disorder: A Consensus Statement of the PTSD Psychopharmacology Working Group. Biol Psychiatry. 82: e51-e59.
Bernardy N C, et al. V A/DoD Clinical Practice Guideline for the Management of Posttraumatic Stress Disorder and Acute Stress Disorder. 2017, Washington, D C: United States Departments of Veterans Affairs and Defense.
Gordon J A, Borja S E, Tuma F K (2017): A Collaborative Psychopharmacology Research Agenda for Posttraumatic Stress Disorder. Biol Psychiatry. 82:460-461.
Shiner B, Drake R E, Watts B V, Desai R A, Schnurr P P (2012): Access to V A services for returning veterans with PTSD. Mil Med. 177:814-822.
Shiner B, Westgate C L, Bernardy N C, Schnurr P P, Watts B V (2017): Anticonvulsant Medication Use in Veterans With Posttraumatic Stress Disorder. J Clin Psychiatry. 78: e545-e552.
Shiner B, Levis M, Dufort V M, Patterson O V, Watts B V, DuVall S L, et al. (2021): Improvements to PTSD quality metrics with natural language processing. J Eval Clin Pract.
Watts B V, Schnurr P P, Mayo L, Young-Xu Y, Weeks W B, Friedman M J (2013): Meta-analysis of the efficacy of treatments for posttraumatic stress disorder. J Clin Psychiatry. 74: e541-550.
APA (2000): Diagnostic and Statistical Manual of Mental Disorders. 4th Edition. Text Revision: DSM-IV-TR. Arlington, VA: American Psychiatric Association.
APA (2013): Diagnostic and Statistical Manual of Mental Disorders: Fifth Edition (DSM-5). Washington, D.C.: American Psychiatric Association.
Blevins C A, Weathers F W, Davis M T, Witte T K, Domino J L (2015): The PTSD Checklist for DSM-5 (PCL-5): Development and Initial Psychometric Evaluation. J Trauma Stress. 28:489-498.
Weathers F W, Litz B T, Herman D S, Huska J A, Keane T M (1993): The PTSD Checklist (PCL): Reliability, validity, and diagnostic utility. Trauma, Coping, and Adaptation. San Antonio, Texas: International Society for Traumatic Stress Studies, 9th Annual Meeting.
Bovin M J, Marx B P, Weathers F W, Gallagher M W, Rodriguez P, Schnurr P P, et al. (2016): Psychometric properties of the PTSD Checklist for Diagnostic and Statistical Manual of Mental Disorders-Fifth Edition (PCL-5) in veterans. Psychol Assess. 28:1379-1391.
Moshier S J, Lee D J, Bovin M J, Gauthier G, Zax A, Rosen R C, et al. (Under review): An empirical crosswalk for the PTSD Checklist: Translating DSM-IV to DSM-5
Lee D J, Bovin M J, Weathers F W, Schnurr P P, Sloan D M, Marx B P (2019): Reliable Change Index and Clinically Significant Margins for the CAPS-5 and PCL-5 among Veterans. Boston, M A: International Society for Traumatic Stress Studies, 35th Annual Meeting.
Jacobson N S, Truax P (1991): Clinical significance: a statistical approach to defining meaningful change in psychotherapy research. J Consult Clin Psychol. 59:12-19.
Bento A P, Gaulton A, Hersey A, Bellis L J, Chambers J, Davies M, et al. (2014): The ChEMBL bioactivity database: an update. Nucleic Acids Res. 42: D1083-1090.
Kulldorff M, Fang Z, Walsh S J (2003): A tree-based scan statistic for database disease surveillance. Biometrics. 59:323-331.
Kulldorff M, Dashevsky I, Avery T R, Chan A K, Davis R L, Graham D, et al. (2013): Drug safety data mining with a tree-based scan statistic. Pharmacoepidemiol Drug Saf. 22:517-523.
Huybrechts K F, Kulldorff M, Hernandez-Diaz S, Bateman B T, Zhu Y, Mogun H, et al. (2021): Active Surveillance of the Safety of Medications Used During Pregnancy. Am J Epidemiol. 190:1159-1168.
Shiner B, Leonard C, Gui J, Cornelius S, Gradus J L, Schnurr P P, et al. (2021): Measurement Strategies for Evidence-Based Antidepressants for Posttraumatic Stress Disorder Delivery: Trends and Associations with Patient-Reported Outcomes. Adm Policy Ment Health. 48:70-87.
Shiner B, Westgate C L, Gui J, Cornelius S, Maguen S E, Watts B V, et al. (2020): Measurement Strategies for Evidence-Based Psychotherapy for Posttraumatic Stress Disorder Delivery: Trends and Associations with Patient-Reported Outcomes. Adm Policy Ment Health. 47:451-467.
The Management of Posttraumatic Stress Disorder Work Group (2017): VA/DoD Clinical Practice Guideline for the Management of Posttraumatic Stress Disorder and Acute Stress Disorder. Washington, DC: US Departments of Veterans Affairs and Defense.
Rosen C S, Matthieu M M, Wiltsey Stirman S, Cook J M, Landes S, Bernardy N C, et al. (2016): A Review of Studies on the System-Wide Implementation of Evidence-Based Psychotherapies for Posttraumatic Stress Disorder in the Veterans Health Administration. Adm Policy Ment Health. 43:957-977.
Terhakopian A, Sinaii N, Engel C C, Schnurr P P, Hoge C W (2008): Estimating population prevalence of posttraumatic stress disorder: an example using the PTSD checklist. J Trauma Stress. 21:290-300.
Hoge C W, Riviere L A, Wilk J E, Herrell R K, Weathers F W (2014): The prevalence of post-traumatic stress disorder (PTSD) in US combat soldiers: a head-to-head comparison of DSM-5 versus DSM-IV-TR symptom criteria with the PTSD checklist. Lancet Psychiatry. 1:269-277.
Huang Z D, Zhao Y F, Li S, Gu H Y, Lin L L, Yang Z Y, et al. (2020): Comparative Efficacy and Acceptability of Pharmaceutical Management for Adults With Post-Traumatic Stress Disorder: A Systematic Review and Meta-Analysis. Front Pharmacol. 11:559.
Stamm T J, Adli M, Kirchheiner J, Smolka M N, Kaiser R, Tremblay P B, et al. (2008): Serotonin transporter gene and response to lithium augmentation in depression. Psychiatr Genet. 18:92-97.
Sofuoglu M, Rosenheck R, Petrakis I (2014): Pharmacological treatment of comorbid PTSD and substance use disorder: Recent progress. Addictive Behaviors. 39:428-433.
Backus L I, Belperio P S, Shahoumian T A, Loomis T P, Mole L A (2017): Real-world effectiveness and predictors of sustained virological response with all-oral therapy in 21,242 hepatitis C genotype-1 patients. Antivir Ther. 22:481-493.
Ioannou G N, Beste L A, Chang M F, Green P K, Lowy E, Tsui J I, et al. (2016): Effectiveness of Sofosbuvir, Ledipasvir/Sofosbuvir, or Paritaprevir/Ritonavir/Ombitasvir and Dasabuvir Regimens for Treatment of Patients With Hepatitis C in the Veterans Affairs National Health Care System. Gastroenterology. 151:457-471 e455.
Zając M, Muszalska I, Sobczak A, Dadej A, Tomczak S, Jelińska A (2019): Hepatitis C-New drugs and treatment prospects. European Journal of Medicinal Chemistry. 165:225-249.
Iriana S, Curry M P, Afdhal N H (2017): Neurologic Manifestations of Hepatitis C Virus Infection. Clin Liver Dis. 21:535-542.
Machado M O, Oriolo G, Bortolato B, Kohler C A, Maes M, Solmi M, et al. (2017): Biological mechanisms of depression following treatment with interferon for chronic hepatitis C: A critical systematic review: J Affect Disord. 209:235-245.
Udina M, Castellvi P, Moreno-Espana J, Navines R, Valdes M, Forns X, et al. (2012): Interferon-induced depression in chronic hepatitis C: a systematic review and meta-analysis. J Clin Psychiatry. 73:1128-1138.
Gallach M, Vergara M, da Costa J P, Miquel M, Casas M, Sanchez-Delgado J, et al. (2018): Impact of treatment with direct-acting antivirals on anxiety and depression in chronic hepatitis C. PLOS One. 13: e0208112.
Hahn D, Stokes C S, Kaiser R, Meyer M R, Lammert F, Gruenhage F (2018): Antidepressant effects of direct-acting antivirals against hepatitis C virus-Results from a pilot study. Eur J Clin Invest. 48: e13024.
Kesen O, Kani H T, Yanartas O, Aykut U E, Gok B, Gunduz F, et al. (2019): Evaluation of depression, anxiety and quality of life in hepatitis C patients who treated with direct acting antiviral agents. Turk J Gastroenterol. 30:801-806.
Pericot-Valverde I, Heo M, Niu J, Norton B L, Akiyama M J, Agyemang L, et al. (2020): Declines in Depressive Symptoms Among People who Inject Drugs Treated With Direct-Acting Antivirals While on Opioid Agonist Therapy. Open Forum Infect Dis. 7: ofaa380.
Wishart D S, Feunang Y D, Guo A C, Lo E J, Marcu A, Grant J R, et al. (2018): DrugBank 5.0: a major update to the DrugBank database for 2018. Nucleic Acids Res. 46: D1074-D1082.
Elbaridi N, Kaye A D, Choi S, Urman R D (2017): Current Concepts of Phenylpiperidine Derivatives Use in the Treatment of Acute and Chronic Pain. Pain Physician. 20: Se23-se31.
van der Kolk B A (1994): The body keeps the score: memory and the evolving psychobiology of posttraumatic stress. Harv Rev Psychiatry. 1:253-265.
Shiner B, Leonard Westgate C, Bernardy N C, Schnurr P P, Watts B V (2017): Trends in Opioid Use Disorder Diagnoses and Medication Treatment Among Veterans With Posttraumatic Stress Disorder. J Dual Diagn. 13:201-212.
He Y, Tan S L, Tareen S U, Vijaysri S, Langland J O, Jacobs B L, et al. (2001): Regulation of mRNA translation and cellular signaling by hepatitis C virus nonstructural protein NS5A. J Virol. 75:5090-5098.
Gal-Ben-Ari S, Barrera I, Ehrlich M, Rosenblum K (2019): PKR: A Kinase to Remember. Frontiers in Molecular Neuroscience. 11.
Zhu P J, Huang W, Kalikulov D, Yoo J W, Placzek A N, Stoica L, et al. (2011): Suppression of PKR promotes network excitability and enhanced cognition by interferon-γ-mediated disinhibition. Cell. 147:1384-1396.
Fonzo G A, Goodkind M S, Oathes D J, Zaiko Y V, Harvey M, Peng K K, et al. (2021): Amygdala and Insula Connectivity Changes Following Psychotherapy for Posttraumatic Stress Disorder: A Randomized Clinical Trial. Biol Psychiatry. 89:857-867.
Belperio P S, Chartier M, Ross D B, Alaigh P, Shulkin D (2017): Curing Hepatitis C Virus Infection: Best Practices From the U.S. Department of Veterans Affairs. Ann Intern Med. 167:499-504.
Mendez D, Gaulton A, Bento A P, Chambers J, De Veij M, Felix E, et al. (2019): ChEMBL: towards direct deposition of bioassay data. Nucleic Acids Res. 47: D930-D940.

### 2. Comparative Effectiveness of Direct-Acting Antivirals for Posttraumatic Stress Disorder

Kessler R C, Chiu W T, Demler O, Merikangas K R, Walters E E. Prevalence, severity, and comorbidity of 12-month DSM-IV disorders in the National Comorbidity Survey Replication. Arch Gen Psychiatry. 2005: 62 (6): 617-27.
Krystal J H, Davis L L, Neylan T C, M A R, Schnurr P P, Stein M B, et al. It Is Time to Address the Crisis in the Pharmacotherapy of Posttraumatic Stress Disorder: A Consensus Statement of the PTSD Psychopharmacology Working Group. Biol Psychiatry. 2017: 82 (7): e51-e59.
DePierro J, Lepow L, Feder A, Yehuda R. Translating Molecular and Neuroendocrine Findings in Posttraumatic Stress Disorder and Resilience to Novel Therapies. Biol Psychiatry: 2019:86 (6): 454-63.
Sartori S B, Singewald N. Novel pharmacological targets in drug development for the treatment of anxiety and anxiety-related disorders. Pharmacol Ther. 2019:204:107402.
Schoenfeld F B, Marmar C R, Neylan T C. Current concepts in pharmacotherapy for posttraumatic stress disorder. Psychiatr Serv. 2004: 55 (5): 519-31.
Yehuda R, Hoge C W, McFarlane A C, Vermetten E, Lanius R A, Nievergelt C M, et al. Post-traumatic stress disorder. Nat Rev Dis Primers. 2015:1:15057.
Raskind M A, Peskind E R, Chow B, Harris C, Davis-Karim A, Holmes H A, et al. Trial of Prazosin for Post-Traumatic Stress Disorder in Military Veterans. N Engl J Med. 2018: 378 (6): 507-17.
Feder A, Costi S, Rutter S B, Collins A B, Govindarajulu U, Jha M K, et al. A Randomized Controlled Trial of Repeated Ketamine Administration for Chronic Posttraumatic Stress Disorder. Am J Psychiatry. 2021: 178 (2): 193-202.
Spangler P T, West J C, Dempsey C L, Possemato K, Bartolanzo D, Aliaga P, et al. Randomized Controlled Trial of Riluzole Augmentation for Posttraumatic Stress Disorder: Efficacy of a Glutamatergic Modulator for Antidepressant-Resistant Symptoms. J Clin Psychiatry: 2020:81 (6).
Ma'ayan A, Jenkins S L, Goldfarb J, Iyengar R. Network analysis of FDA approved drugs and their targets. Mt Sinai J Med. 2007: 74 (1): 27-32.
Barneh F, Jafari M, Mirzaie M. Updates on drug-target network: facilitating polypharmacology and data integration by growth of DrugBank database. Brief Bioinform. 2016: 17 (6): 1070-80.
Gordon J A, Borja S E, Tuma F K. A Collaborative Psychopharmacology Research Agenda for Posttraumatic Stress Disorder. Biol Psychiatry. 2017: 82 (7): 460-61.
Shiner B, Forehand J A, Rozema L, Kulldorff M, Watts B V, Trefethen M, et al. Mining Clinical Data for Novel PTSD Medications. Biol Psychiatry. 2021: (Online First: DOI: https://doi.org/10.1016/j.biopsych 2021.10.008.
Belperio P S, Chartier M, Ross D B, Alaigh P, Shulkin D. Curing Hepatitis C Virus Infection: Best Practices From the U.S. Department of Veterans Affairs. Ann Intern Med. 2017: 167 (7): 499-504.
Zajac M, Muszalska I, Sobczak A, Dadej A, Tomczak S, Jelinska A. Hepatitis C-New drugs and treatment prospects. Eur J Med Chem. 2019:165:225-49.
Shiner B, Watts B V. Baby Steps to a Learning Mental Health Care System: Can we do the Work? Am J Epidemiol. 2020.
Shiner B, Leonard C E, Gui J, Cornelius S L, Schnurr P P, Hoyt J E, et al. Comparing Medications for DSM-5 PTSD in Routine V A Practice. J Clin Psychiatry. 2020: 81 (6).
Shiner B, Westgate C L, Gui J, Maguen S, Young-Xu Y, Schnurr P P, et al. A Retrospective Comparative Effectiveness Study of Medications for Posttraumatic Stress Disorder in Routine Practice. J Clin Psychiatry. 2018: 79 (5).
Shiner B, Gui J, Rozema L, Cornelius S L, Dufort V, Schnurr P P, et al. Patient and Clinical Factors Associated With Response to Medications for Posttraumatic Stress Disorder. J Clin Psychiatry. 2021: 82 (6).
Holder N, Shiner B, Li Y, Madden E, Neylan T C, Seal K H, et al. Cognitive Processing Therapy for Veterans with Posttraumatic Stress Disorder: What is the Median Effective Dose? J Affect Disord. 2020:273:425-33.
Holder N, Shiner B, Li Y, Madden E, Neylan T C, Seal K H, et al. Determining the median effective dose of prolonged exposure therapy for veterans with posttraumatic stress disorder. Behav Res Ther. 2020:135:103756.
Shiner B, Levis M, Dufort V M, Patterson O V, Watts B V, DuVall S L, et al. Improvements to PTSD quality metrics with natural language processing. J Eval Clin Pract. 2021.
American Psychiatric Association. Diagnostic and Statistical Manual of Mental Disorders: Fifth Edition (DSM-5). American Psychiatric Association: Washington, D.C.: 2013.
American Psychiatric Association. Diagnostic and Statistical Manual of Mental Disorders, 4th Edition, Text Revision: DSM-IV-TR American Psychiatric Association: Arlington, VA: 2000.
Blevins C A, Weathers F W, Davis M T, Witte T K, Domino J L. The PTSD Checklist for DSM-5 (PCL-5): Development and Initial Psychometric Evaluation. J Trauma Stress. 2015: 28 (6): 489-98.
Weathers F W, Litz B T, Herman D S, Huska J A, Keane T M. in Trauma, Coping, and Adaptation (International Society for Traumatic Stress Studies, 9th Annual Meeting, San Antonio, Texas, 1993).
Bovin M J, Marx B P, Weathers F W, Gallagher M W, Rodriguez. P, Schnurr P P, et al. Psychometric properties of the PTSD Checklist for Diagnostic and Statistical Manual of Mental Disorders-Fifth Edition (PCL-5) in veterans. Psychol Assess. 2016: 28 (11): 1379-91.
Moshier S J, Lee D J, Bovin M J, Gauthier G, Zax A, Rosen R C, et al. An Empirical Crosswalk for the PTSD Checklist: Translating DSM-IV to DSM-5 Using a Veteran Sample. J Trauma Stress. 2019: 32 (5): 799-805.
Marx B P, Lee D J, Norman S B, Bovin M J, Sloan D M, Weathers F W, et al. Reliable and Clinically Significant Change in the Clinician-Administered PTSD Scale for DSM-5 and PTSD Checklist for DSM-5 among Male Veterans. Psychol Assess. 2021: (In Press).
Maguen S, Madden E, Patterson O V, DuVall S L, Goldstein L A, Burkman K, et al. Measuring Use of Evidence Based Psychotherapy for Posttraumatic Stress Disorder in a Large National Healthcare System. Adm Policy Ment Health. 2018: 45 (4): 519-29.
Shiner B, Peltzman T, Cornelius S L, Gui J, Forehand J, Watts B V. Recent trends in the rural-urban suicide disparity among veterans using V A health care. J Behav Med. 2021: 44 (4): 492-506.
Sterling R K, Lissen E, Clumeck N, Sola R, Correa M C, Montaner J, et al. Development of a simple noninvasive index to predict significant fibrosis in patients with HIV/HCV coinfection. Hepatology. 2006: 43 (6): 1317-25.
Vallet-Pichard A, Mallet V, Nalpas B, Verkarre V, Nalpas A, Dhalluin-Venier V,
et al. FIB-4: an inexpensive and accurate marker of fibrosis in HCV infection, comparison with liver biopsy and fibrotest. Hepatology: 2007:46 (1): 32-6.
The Management of Posttraumatic Stress Disorder Work Group. VA/DoD CPG for the Management of PTSD and ASD. United States Departments of Veterans Affairs and Defense: Washington, DC: 2017.
Lash T L, VanderWeele T J, Haneuse S, Rothman K J. Modern Epidemiology, Fourth Edition. Wolters Kluwer: New York: 2020.
Stuart E A. Matching methods for casual inference: a review and a look forward. Statistical Science. 2010: 25 (1): 1-21.
McCaffrey D F, Griffin B A, Almirall D, Slaughter M E, Ramchand R, Burgette L F. A tutorial on propensity score estimation for multiple treatments using generalized boosted models. Statistics in medicine. 2013: 32 (19): 3388-414.
Wilkinson J, Radkowski M, Eschbacher J M, Laskus T. Activation of brain macrophages/microglia cells in hepatitis C infection. Gut. 2010: 59 (10): 1394-400.
Adinolfi L E, Nevola R, Lus G, Restivo L, Guerrera B, Romano C, et al. Chronic hepatitis C virus infection and neurological and psychiatric disorders: an overview. World J Gastroenterol. 2015: 21 (8): 2269-80.
Stabinski L, Hodowenec A. NDA 209394 Clinical Review: Mavyret (glecaprevir and pibrentasvir). United States Food and Drug Administration: Washington, D C: 2015.
Bhatt S, Hillmer A T, Girgenti M J, Rusowicz A, Kapinos M, Nabulsi N, et al. PTSD is associated with neuroimmune suppression: evidence from PET imaging and postmortem transcriptomic studies. Nature Communications. 2020: 11 (1): 2360.
Kim T D, Lee S, Yoon S. Inflammation in Post-Traumatic Stress Disorder (PTSD): A Review of Potential Correlates of PTSD with a Neurological Perspective. Antioxidants (Basel). 2020: 9 (2): 107.
Hori H, Kim Y. Inflammation and post-traumatic stress disorder. Psychiatry and Clinical Neurosciences. 2019: 73 (4): 143-53.
McGivern D R, Masaki T, Lovell W, Hamlett C, Saalau-Bethell S, Graham B, et al. Protease Inhibitors Block Multiple Functions of the NS3/4A Protease-Helicase during the Hepatitis C Virus Life Cycle. Journal of Virology. 2015: 89 (10): 5362-70.
Rivas-Estilla A M, Svitkin Y, Lastra M L, Hatzoglou M, Sherker A, Koromilas A E. PKR-Dependent Mechanisms of Gene Expression from a Subgenomic Hepatitis C Virus Clone. Journal of Virology. 2002: 76 (21): 10637-53.
Coulombe J, Moodie EEM, Shortreed S M, Renoux C. Coulombe et al. Respond to “Baby Steps to a Learning Mental Health-Care System”. Am J Epidemiol. 2021: 190 (7): 1223-24.
Austin P C. Balance diagnostics for comparing the distribution of baseline covariates between treatment groups in propensity-score matched samples. Statistics in medicine. 2009: 28 (25): 3083-107.
   3. Changes in Alcohol Consumption Following Direct-Acting Antiviral Treatment for Hepatitis C in Va Patients with Comorbid Alcohol Use Disorder and Ptsd
Anand, B. S., Currie, S., Dieperink, E., Bini, E. J., Shen, H., Ho, S. B., & Wright, T. (2006). Alcohol use and treatment of hepatitis C virus: results of a national multicenter study: Gastroenterology. 130 (6), 1607-1616. https://doi.org/10.1053/j.gastro.2006.02.023
Austin, P. C. (2009). Balance diagnostics for comparing the distribution of baseline covariates between treatment groups in propensity-score matched samples. Statistics in Medicine. 28 (25), 3083-3107. https://doi.org/10.1002/sim.3697
Barua, S., Greenwald, R., Grebely, J., Dore, G. J., Swan, T., & Taylor, L. E. (2015). Restrictions for Medicaid reimbursement of sofosbuvir for the treatment of hepatitis C virus infection in the United States. Annals of Internal Medicine. 163 (3), 215-223. https://doi.org/10.7326/m15-0406
Belperio, P. S., Chartier, M., Ross, D. B., Alaigh, P., & Shulkin, D. (2017). Curing hepatitis C virus infection: Best practices from the U.S. Department of Veterans Affairs. Annals of Internal Medicine. 167 (7), 499-504. https://doi.org/10.7326/M17-1073
Bradley, K. A., Kivlahan, D. R., & Williams, E. C. (2009). Brief approaches to alcohol screening: practical alternatives for primary care. Journal of General Internal Medicine. 24 (7), 881-883. https://doi.org/10.1007/s11606-009-1014-9
Breen, M. S., Maihofer, A. X., Glatt, S. J., Tylee, D. S., Chandler, S. D., Tsuang, M. T., Risbrough, V. B., Baker, D. G., O'Connor, D. T., Nievergelt, C. M., & Woelk, C. H. (2015). Gene networks specific for innate immunity define post-traumatic stress disorder. Molecular Psychiatry. 20 (12), 1538-1545. https://doi.org/10.1038/mp.2015.9
Bush, K., Kivlahan, D. R., McDonell, M. B., Fihn, S. D., & Bradley, K. A. (1998). The AUDIT alcohol consumption questions (AUDIT-C): an effective brief screening test for problem drinking. Alcohol Use Disorders Identification Test. Archives of Internal Medicine. 158 (16), 1789-1795. https://doi.org/10.1001/archinte. 158.16.1789
Butt, A. A., Justice, A. C., Skanderson, M., Rigsby, M. O., Good, C. B., & Kwoh, C. K. (2007). Rate and predictors of treatment prescription for hepatitis C. Gut. 56 (3), 385-389. https://doi.org/10.1136/gut.2006.099150
Casella, G., Rasouli, J., Mason, K., Boehm, A., Kumar, G., Hwang, D., Thome, R., Ishikawa, L., Zhang, G.-X., Ciric, B., & Rostami, A. (2020). A serine protease inhibitor suppresses autoimmune neuroinflammation by activating the STING/IFN-β axis in macrophages. Cellular & Molecular Immunology. 17 (12), 1278-1280. https://doi.org/10.1038/s41423-020-0405-z
çelik, S., KayaaltΘ, A., & Erbas, O. (2022). NLRP3 Inflammasome: A new target in psychiatric disorders. CNS Neuroscience and Theraputics. 2, 392-398. https://doi.org/10.5606/jebms.2021.75681
Chang, A., Skole, K., Gautam, M., Schmutz, J., Black, M., Thomas, R., Horwitz, B., & Friedenberg, F. (2005). The impact of past alcohol use on treatment response rates in patients with chronic hepatitis C. Alimentary Pharmacology & Therapeutics. 22 (8), 701-706. https://doi.org/10.1111/j.1365-2036.2005.02631.x
Chen, W., Xu, Y., Li, H., Tao, W., Xiang, Y., Huang, B., Niu, J., Zhong, J., & Meng, G. (2014). HCV genomic RNA activates the NLRP3 inflammasome in human myeloid cells. PloS one. 9 (1), e84953. https://doi.org/10.1371/journal.pone.0084953
Cheon, S. Y., Kim, J., Kim, S. Y., Kim, E. J., & Koo, B.-N. (2020). Inflammasome and cognitive symptoms in human diseases: biological evidence from experimental research. International Journal of Molecular Sciences. 21 (3), 1103.https://doi.org/10.3390/ijms21031103
Debell, F., Fear, N. T., Head, M., Batt-Rawden, S., Greenberg, N., Wessely, S., & Goodwin, L. (2014). A systematic review of the comorbidity between PTSD and alcohol misuse. Social Psychiatry and Psychiatric Epidemiology. 49 (9), 1401-1425. https://doi.org/10.1007/s00127-014-0855-7
Dong, Y., Li, S., Lu, Y., Li, X., Liao, Y., Peng, Z., Li, Y., Hou, L., Yuan, Z., & Cheng, J. (2020). Stress-induced NLRP3 inflammasome activation negatively regulates fear memory in mice. Journal of Neuroinflammation. 17 (1), 205. https://doi.org/10.1186/s12974-020-01842-0
Fusco, R., Siracusa, R., Genovese, T., Cuzzocrea, S., & Di Paola, R. (2020). Focus on the role of NLRP3 inflammasome in diseases. International Journal of Molecular Sciences. 21 (12). https://doi.org/10.3390/ijms21124223
Gal-Ben-Ari, S., Barrera, I., Ehrlich, M., & Rosenblum, K. (2019). PKR: A kinase to remember [Review]. Frontiers in Molecular Neuroscience. 11. https://doi.org/10.3389/fnmol.2018.00480
Giovannini, C., Fornari, F., Indio, V., Treré, D., Renzulli, M., Vasuri, F., Cescon, M., Ravaioli, M., Perrucci, A., Astolfi, A., Piscaglia, F., & Gramantieri, L. (2020). Direct antiviral treatments for hepatitis C virus have off-target effects of oncologic relevance in hepatocellular carcinoma. Cancers. 12 (9). https://doi.org/10.3390/cancers12092674
Gupta, S. C., Sundaram, C., Reuter, S., & Aggarwal, B. B. (2010). Inhibiting N F-K B activation by small molecules as a therapeutic strategy. Biochimica et Biophysica Acta. 1799 (10-12), 775-787. https://doi.org/10.1016/j.bbagrm.2010.05.004
He, Y., Tan, S.-L., Tareen, S. U., Vijaysri, S., Langland, J. O., Jacobs, B. L., & Katze, M. G. (2001). Regulation of mRNA translation and cellular signaling by hepatitis C virus nonstructural protein NS5A. Journal of Virology. 75 (11), 5090-5098. https://doi.org/doi: 10.1128/JVI.75.11.5090-5098.2001
Herink, M. C., Geddes, J., Vo, K., Zaman, A., & Hartung, D. M. (2021). Effect of relaxing hepatitis C treatment restrictions on direct-acting antiviral use in a Medicaid program: an interrupted time series analysis. Journal of Managed Care & Specialty Pharmacy. 27 (7), 856-864.
Hoyt, L. R., Ather, J. L., Randall, M. J., DePuccio, D. P., Landry, C. C., Wewers, M. D., Gavrilin, M. A., & Poynter, M. E. (2016). Ethanol and other short-chain alcohols inhibit NLRP3 inflammasome activation through protein tyrosine phosphatase stimulation. Journal of Immunology. 197 (4), 1322-1334. https://doi.org/10.4049/jimmunol. 1600406
Infectious Diseases Society of America (2021). HCV Guidance: Recommendations for Testing. Managing. and Treating Hepatis C. https://www.hcvguidelines.org/
Jackson, W. E., & Everson, G. T. (2017). Sofosbuvir and velpatasvir for the treatment of hepatitis C. Expert Review of Rastroenterology & Hepatology. 11 (6), 501-505.
Kapil, P., Stohlman, S. A., Hinton, D. R., & Bergmann, C. C. (2014). PKR mediated regulation of inflammation and IL-10 during viral encephalomyelitis. Journal of Neuroimmunology. 270 (1-2), 1-12. https://doi.org/10.1016/j.jneuroim.2014.02.012
Kessler, R. C., Sonnega, A., Bromet, E., Hughes, M., & Nelson, C. B. (1995). Posttraumatic stress disorder in the National Comorbidity Survey. Archives of Geneneral Psychiatry. 52 (12), 1048-1060. https://doi.org/10.1001/archpsyc. 1995.03950240066012
Khan, A., Tansel, A., White, D. L., Kayani, W. T., Bano, S., Lindsay, J., El-Serag. H. B., & Kanwal, F. (2016). Efficacy of psychosocial interventions in inducing and maintaining alcohol abstinence in patients with chronic liver disease: A Systematic Review. Clinical Gastroenterology and Hepatology. 14 (2), 191-e120. https://doi.org/10.1016/j.cgh.2015.07.047
Kim, N. J., Pearson, M., Vutien, P., Su, F., Moon, A. M., Berry, K., Green, P. K., Williams, E. C., & Ioannou, G. N. (2020). Alcohol use and long-term outcomes among U.S. veterans who received direct-acting antivirals for hepatitis C treatment. Hepatology Communications. 4 (2), 314-324. https://doi.org/10.1002/hep4.1464
Kowdley, K. V., Gordon, S. C., Reddy, K. R., Rossaro, L., Bernstein, D. E., Lawitz, E., Shiffman, M. L., Schiff, E., Ghalib, R., & Ryan, M. (2014). Ledipasvir and sofosbuvir for 8 or 12 weeks for chronic HCV without cirrhosis. New England Journal of Medicine. 370 (20), 1879-1888.
Kranzler, H. R., & Soyka, M. (2018). Diagnosis and pharmacotherapy of alcohol use disorder: A Review. Journal of the American Medical Association. 320 (8), 815-824. https://doi.org/10.1001/jama.2018.11406
Kwako, L. E., Schwandt, M. L., Sells, J. R., Ramchandani, V. A., Hommer, D. W., George, D. T., Sinha, R., & Heilig, M. (2015). Methods for inducing alcohol craving in individuals with co-morbid alcohol dependence and posttraumatic stress disorder: behavioral and physiological outcomes. Addiction Biology. 20 (4), 733-746. https://doi.org/10.1111/adb.12150
Lamb, Y. N. (2017). Glecaprevir/pibrentasvir: first global approval. Drugs. 77 (16), 1797-1804. https://doi.org/10.1007/s40265-017-0817-y
Lan, T., Tao, A., Xu, X., Kvietys, P., & Rui, T. (2020). Peroxynitrite/PKR axis modulates the NLRP3 inflammasome of cardiac fibroblasts. Frontiers in Immunology. 11, 558712. https://doi.org/10.3389/fimmu.2020.558712
Lowe, P. P., Cho, Y., Tornai, D., Coban, S., Catalano, D., & Szabo, G. (2020). Inhibition of the inflammasome signaling cascade reduces alcohol consumption in female but not male mice. Alcoholism. 44 (2), 567-578. https://doi.org/10.1111/acer.14272
Lu, B., Nakamura, T., Inouye, K., Li, J., Tang, Y., Lundbäck, P., Valdes-Ferrer, S. I., Olofsson, P. S., Kalb, T., Roth, J., Zou, Y., Erlandsson-Harris, H., Yang, H., Ting, J. P., Wang, H., Andersson, U., Antoine, D. J., Chavan, S. S., Hotamisligil, G. S., & Tracey, K. J. (2012). Novel role of PKR in inflammasome activation and HMGBI release. Nature. 488 (7413), 670-674. https://doi.org/10.1038/naturel1290
Maguen, S., Holder, N., Madden, E., Li, Y., Seal, K. H., Neylan, T. C., Lujan, C., Patterson, O. V., DuVall, S. L., & Shiner, B. (2020). Evidence-based psychotherapy trends among posttraumatic stress disorder patients in a national healthcare system, 2001-2014. Depression and Anxiety. 37 (4), 356-364. https://doi.org/10.1002/da.22983
Maguen, S., Madden, E., Patterson, O. V., DuVall, S. L., Goldstein, L. A., Burkman, K., & Shiner, B. (2018). Measuring use of evidence based psychotherapy for posttraumatic stress disorder in a large national healthcare system. Administration and Policy in Mental Health and Mental Health Services Research. 45 (4), 519-529. https://doi.org/10.1007/s10488-018-0850-5
Management of Posttraumatic Stress Disorder Work Group (2017). A/DOD Clinical Practice Guideline for the Management of Posttraumatic Stress Disorder and Acute Stress Disorder. U.S. Department of Veterans Affairs. https://www.healthquality.va.gov/guidelines/MH/ptsd/VADoDPTSDCPGFinal012418.pdf Marshall, C. C., S: Szabo, G. (2021). Hepatitis C and alcohol. Retrieved February 24 from https://www.uptodate.com/contents/hepatitis-c-and-alcohol?search=hepatitis % 20c %20 and %20alcohol&source=search_result&selectedTitle=1˜1 50&usage_type=default&display_rank=1
McCaffrey, D. F., Griffin, B. A., Almirall, D., Slaughter, M. E., Ramchand, R., & Burgette, L. F. (2013). A tutorial on propensity score estimation for multiple treatments using generalized boosted models. Statistics in Medicine. 32 (19), 3388-3414. https://doi.org/10.1002/sim.5753
McLean, C. P., Su, Y. J., & Foa, E. B. (2015). Mechanisms of symptom reduction in a combined treatment for comorbid posttraumatic stress disorder and alcohol dependence. Journal of Consulting and Clinical Psychology. 83 (3), 655-661. https://doi.org/10.1037/ccp0000024
Memon, M. I., & Memon, M. A. (2002). Hepatitis C: An epidemiological review. Journal of Viral Hepatitis. 9 (2), 84-100. https://doi.org/https://doi.org/10.1046/j.1365-2893.2002.00329.x
Nennig, S. E., & Schank, J. R. (2017). The role of NFKB in drug addiction: Beyond inflammation. Alcohol and Alcoholism. 52 (2), 172-179. https://doi.org/10.1093/alcalc/agw098
Okazaki, T., Yoshihara, H., Suzuki, K., Yamada, Y., Tsujimura, T., Kawano, K., Yamada, Y., & Abe, H. (1994). Efficacy of interferon therapy in patients with chronic hepatitis C: Comparison between non-drinkers and drinkers. Scandinavian Journal of Gastroenterology. 29 (11), 1039-1043. https://doi.org/10.3109/00365529409094883
Owens, M. D., Ioannou, G. N., Tsui, J. L., Edelman, E. J., Greene, P. A., & Williams, E. C. (2018). Receipt of alcohol-related care among patients with HCV and unhealthy alcohol use. Drug and Alcohol Dependence. 188, 79-85. https://doi.org/10.1016/j.drugalcdep.2018.03.047
Pietrzak, R. H., Goldstein, R. B., Southwick, S. M., & Grant, B. F. (2011). Prevalence and Axis I comorbidity of full and partial posttraumatic stress disorder in the United States: results from Wave 2 of the National Epidemiologic Survey on Alcohol and Related Conditions. Journal of Anxiety Disorders. 25 (3), 456-465. https://doi.org/10.1016/j.janxdis.2010.11.010
Price, L. E., Shea, K., & Gephart, S. (2015). The Veterans Affairs's Corporate Data Warehouse: Uses and implications for nursing research and practice. Nursing Administration Quarterly. 39 (4), 311-318. https://doi.org/10.1097/NAQ.0000000000000118
Proeschold-Bell, R. J., Evon, D. M., Yao, J., Niedzwiecki, D., Makarushka, C., Keefe, K. A., Patkar, A. A., Mannelli, P., Garbutt, J. C., Wong, J. B., Wilder, J. M., Datta, S. K., Hodge, T., Naggie, S., Fried, M. W., & Muir, A. J. (2020). A randomized controlled trial of an integrated alcohol reduction intervention in patients with hepatitis C infection. Hepatology. 71 (6), 1894-1909. https://doi.org/10.1002/hep.31058
Ramachandran, A., Kumar, B., Waris, G., & Everly, D. (2021). Deubiquitination and activation of the NLRP3 inflammasome by UCHL5 in HCV-infected cells. Microbiology Spectrum. 9 (1), e0075521. https://doi.org/10.1128/Spectrum.00755-21
Rentsch, C. T., Cartwright, E. J., Gandhi, N. R., Brown, S. T., Rodriguez-Barradas, M. C., Goetz, M. B., Marconi, V. C., Gibert, C. L., Re, V. L., 3rd, Fiellin, D. A., Justice, A. C., & Tate, J. P. (2018). Provider verification of electronic health record receipt and nonreceipt of direct-acting antivirals for the treatment of hepatitis C virus infection. Annals of Epidemiology. 28 (11), 808-811. https://doi.org/10.1016/j.annepidem.2018.08.007
Rubinsky, A. D., Dawson, D. A., Williams, E. C., Kivlahan, D. R., & Bradley, K. A. (2013). AUDIT-C scores as a scaled marker of mean daily drinking, alcohol use disorder severity, and probability of alcohol dependence in a U.S. general population sample of drinkers. Alcoholism. 37 (8), 1380-1390. https://doi.org/10.1111/acer.12092
Seal, K. H., Cohen, G., Bertenthal, D., Cohen, B. E., Maguen, S., & Daley, A. (2011). Reducing barriers to mental health and social services for Iraq and Afghanistan veterans: outcomes of an integrated primary care clinic. Journal of General Internal Medicine. 26 (10), 1160-1167. https://doi.org/10.1007/s11606-011-1746-1
Seal, K. H., Cohen, G., Waldrop, A., Cohen, B. E., Maguen, S., & Ren, L. (2011). Substance use disorders in Iraq and Afghanistan veterans in VA healthcare, 2001-2010: Implications for screening, diagnosis and treatment. Drug and Alcohol Dependence. 116 (1-3), 93-101. https://doi.org/10.1016/j.drugalcdep.2010.11.027
Sharma, B. R., & Kanneganti, T.-D. (2021). NLRP3 inflammasome in cancer and metabolic diseases. Nature Immunology. 22 (5), 550-559. https://doi.org/10.1038/s41590-021-00886-5
Shiner, B., Forehand, J. A., Rozema, L., Kulldorff, M., Watts, B. V., Trefethen, M., Jiang. T., Huybrechts, K. F., Schnurr, P. P., Vincenti, M., Gui, J., & Gradus, J. L. (2022). Mining clinical data for novel posttraumatic stress disorder medications. Biological Psychiatry. 91 (7), 647-657. https://doi.org/10.1016/j.biopsych.2021.10.008
Shiner, B., Huybrechts, K., Gui, J., Rozema, L., Forehand, J., Watts, B. V., Jiang, T., Hoyt, J. E., Esteves, J., Schnurr, P. P., Ray, K., & Gradus, J. L. (2022). Comparative effectiveness of direct-acting antivirals for posttraumatic stress disorder in VA patients with hepatitis C virus infection. American Journal of Epidemiology. (In Press). https://doi.org/10.1093/aje/kwac104
Shiner, B., Leonard Westgate, C., Simiola, V., Thompson, R., Schnurr, P. P., & Cook, J. M. (2018). Measuring use of evidence-based psychotherapy for PTSD in VA residential treatment settings with clinician survey and electronic medical record templates. Military Medicine. 183 (9-10), e539-e546. https://doi.org/10.1093/milmed/usy008
Shiner, B., Peltzman, T., Cornelius, S. L., Gui, J., Forehand, J., & Watts, B. V. (2021). Recent trends in the rural-urban suicide disparity among veterans using VA health care. Journal of Behavioral Medicine. 44 (4), 492-506. https://doi.org/10.1007/s10865-020-00176-9
Shiner, B., Tang, C., Trapp, A. C., Konrad, R., Bar-On, I., & Watts, B. V. (2014). The provision of mental health treatment after screening: exploring the relationship between treatment setting and treatment intensity General Hospital Psychiatry. 36 (6), 581-588. https://doi.org/10.1016/j.genhosppsych.2014.07.009
Singal, A. K., & Anand, B. S. (2007). Mechanisms of synergy between alcohol and hepatitis C virus. Journal of Clinical Gastroenterology. 41 (8), 761-772. https://doi.org/10.1097/MCG.0b013e3180381584
Sterling, R. K., Lissen, E., Clumeck, N., Sola, R., Correa, M. C., Montaner, J., M, S. S., Torriani, F. J., Dieterich, D. T., Thomas, D. L., Messinger, D., Nelson, M., & Investigators, A. C. (2006). Development of a simple noninvasive index to predict significant fibrosis in patients with HIV/HCV coinfection. Hepatology. 43 (6), 1317-1325. https://doi.org/10.1002/hep.21178
Stuart, E. A. (2010). Matching methods for casual inference: a review and a look forward. Statistical Science. 25 (1), 1-21. https://doi.org/10.1214/09-STS313
Suzuki, R., Matsuda, M., Shimoike, T., Watashi, K., Aizaki, H., Kato, T., Suzuki, T., Muramatsu, M., & Wakita, T. (2019). Activation of protein kinase R by hepatitis C virus RNA-dependent RNA polymerase. Virology. 529, 226-233. https://doi.org/10.1016/j.virol.2019.01.024
Swift, R. M., & Aston, E. R. (2015). Pharmacotherapy for alcohol use disorder: current and emerging therapies. Harvard Review of Psychiatry. 23 (2), 122-133. https://doi.org/10.1097/HRP.0000000000000079
Tsui, J. I., Williams, E. C., Green, P. K., Berry, K., Su, F., & Ioannou, G. N. (2016). Alcohol use and hepatitis C virus treatment outcomes among patients receiving direct antiviral agents. Drug and Alcohol Dependence. 169, 101-109. https://doi.org/10.1016/j.drugalcdep.2016.10.021
Tucker, J. A., Chandler, S. D., & Witkiewitz, K. (2020). Epidemiology of recovery from alcohol use disorder. Alcohol Research. 40 (3), 02. https://doi.org/10.35946/arcr.v40.3.02
U.S. Department of Veterans Affairs. (2016). VA Expands Hepatitis C Drug Treatment [Press release]. https://www.va.gov/opa/pressrel/pressrelease.cfm?id=2762
Vallet-Pichard, A., Mallet, V., Nalpas, B., Verkarre, V., Nalpas, A., Dhalluin-Venier, V., Fontaine, H., & Pol, S. (2007). FIB-4: an inexpensive and accurate marker of fibrosis in HCV infection, comparison with liver biopsy and fibrotest. Hepatology. 46 (1), 32-36. https://doi.org/10.1002/hep.21669
Wang, Z., Zhang, S., Xiao, Y., Zhang, W., Wu, S., Qin, T., Yue, Y., Qian, W., & Li, L. (2020). NLRP3 inflammasome and inflammatory diseases. Oxidative Medicine and Cellular Longevity. 2020, 4063562. https://doi.org/10.1155/2020/4063562
Watkins, L. E., Sprang, K. R., & Rothbaum, B. O. (2018). Treating PTSD: A review of evidence-based psychotherapy interventions [Review]. Frontiers in Behavioral Neuroscience. 12. https://doi.org/10.3389/fnbeh.2018.00258
Westbrook, R. H., & Dusheiko, G. (2014). Natural history of hepatitis C. Journal of Hepatology. 61 (1, Supplement), S58-S68. https://doi.org/https://doi.org/10.1016/j.jhep.2014.07.012
Wohleb, E. S., McKim, D. B., Sheridan, J. F., & Godbout, J. P. (2014). Monocyte trafficking to the brain with stress and inflammation: a novel axis of immune-to-brain communication that influences mood and behavior. Frontiers in Neuroscience. 8, 447. https://doi.org/10.3389/fnins.2014.00447
Yamanashi, T., Iwata, M., Shibushita, M., Tsunetomi, K., Nagata, M., Kajitani, N., Miura, A., Matsuo, R., Nishiguchi, T., Kato, T. A., Setoyama, D., Shirayama, Y., Watanabe, K., Shinozaki, G., & Kaneko, K. (2020). Beta-hydroxybutyrate, an endogenous NLRP3 inflammasome inhibitor, attenuates anxiety-related behavior in a rodent post-traumatic stress disorder model. Scientific Reports. 10 (1), 21629. https://doi.org/10.1038/s41598-020-78410-2
Zhang, L., Alter, H. J., Wang, H., Jia, S., Wang, E., Marincola, F. M., Shih, J. W., & Wang, R. Y. (2013). The modulation of hepatitis C virus 1a replication by PKR is dependent on NF-κB mediated interferon beta response in Huh7.5.1 cells. Virology. 438 (1), 28-36. https://doi.org/10.1016/j.virol.2013.01.015

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A method of treating a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the subject has not been diagnosed as having hepatitis C virus.

2. The method of claim 1, wherein the method comprises administering an individually effective amount of one or more compounds selected from pibrentasvir, glecaprevir, and velpatasvir, or pharmaceutically acceptable salts thereof.

3. The method of claim 1, wherein the method comprises administering a synergistically effective amount of two or more compounds selected from pibrentasvir, glecaprevir, and velpatasvir, or pharmaceutically acceptable salts thereof.

4. The method of claim 1, wherein the method comprises administering exactly one of pibrentasvir, glecaprevir, and velpatasvir, or pharmaceutically acceptable salts thereof.

5. The method of claim 1, wherein the method comprises administering pibrentasvir or a pharmaceutically acceptable salt thereof.

6. The method of claim 1, wherein the method comprises administering glecaprevir or a pharmaceutically acceptable salt thereof.

7. The method of claim 1, wherein the method comprises administering velpatasvir or a pharmaceutically acceptable salt thereof.

8. The method of claim 1, wherein the method comprises administering pibrentasvir and glecaprevir.

9-10. (canceled)

11. The method of claim 0, wherein pibrentasvir and glecaprevir are co-formulated.

12. The method of claim 0, wherein the ratio of pibrentasvir to glecaprevir is of from about 1:10 to about 4:7.

13-15. (canceled)

16. The method of claim 11, wherein pibrentasvir and glecaprevir are co-formulated as a single oral dosage form.

17. The method of claim 1, wherein the one or more compounds are administered orally.

18. (canceled)

19. The method of claim 1, wherein the psychiatric disorder is a trauma- or stressor-related disorder.

20. (canceled)

21. The method of claim 1, wherein the psychiatric disorder is a substance-use disorder.

22-25. (canceled)

26. The method of claim 1, wherein the subject is not currently undergoing treatment for hepatitis C infection.

27. The method of claim 0, wherein the subject is not currently undergoing treatment for an infection due to a flavivirus.

28. The method of claim 1, wherein the subject has been diagnosed with a need for treatment of the psychiatric disorder under Diagnostic and Statistical Manual of Mental Disorders (DSM-V) guidelines.

29-38. (canceled)

39. A method of reducing the severity of one or more symptoms of a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the subject has not been diagnosed as having hepatitis C virus.

40. The method of claim 0, wherein the symptoms are selected from irritability, anger, sadness, depression, and dysphoria.

41. A method of treating a psychiatric disorder in a subject in need thereof, the method comprising administering to the subject an effective amount of one or more compounds selected from pibrentasvir, glecaprevir, and velpatasvir, or pharmaceutically acceptable salts thereof, wherein the psychiatric disorder is one or more selected from attention-deficit/hyperactivity disorder, autism spectrum disorder, conduct disorder, disruptive mood dysregulation disorder, an eating disorder, gender dysphoria, internet gaming disorder, obsessive-compulsive disorder, a paraphilic disorder, a personality disorder, posttraumatic stress disorder, a sleep-wake disorder, a specific learning disorder, a social communication disorder, a substance-use disorder, and a somatic symptom disorder.

42-95. (canceled)
